SEQUENCE LISTING (148 sequences)

SEQ ID NO: 1 - protein, 21 aa, Artificial sequence
  Synthetic construct
ATVVIVVCVSFLVFMIILGVF

SEQ ID NO: 2 - protein, 22 aa, Artificial sequence
  Synthetic construct
IATVVIIISVCMLVFVVAMGVY

SEQ ID NO: 3 - protein, 23 aa, Artificial sequence
  Synthetic construct
AVSGLLIMGAGGGSLIVLSMLLL

SEQ ID NO: 4 - protein, 24 aa, Artificial sequence
  Synthetic construct
SALIGLLVIAVAIATVIVISLVML

SEQ ID NO: 5 - protein, 22 aa, Artificial sequence
  Synthetic construct
VIGIIVPIVVIALLCMSGYLIW

SEQ ID NO: 6 - protein, 24 aa, Artificial sequence
  Synthetic construct
GAIIGLMVGGVVIATVIVITLVML

SEQ ID NO: 7 - protein, 24 aa, Artificial sequence
  Synthetic construct
ILVICLIAVMVVFIILVIGVCTCC

SEQ ID NO: 8 - protein, 23 aa, Artificial sequence
  Synthetic construct
MGIAFAAFVIGALLTGALWYIYS

SEQ ID NO: 9 - protein, 24 aa, Artificial sequence
  Synthetic construct
GMLPVAVLVALLAVIVLVALLLLW

SEQ ID NO: 10 - protein, 23 aa, Artificial sequence
  Synthetic construct
WLIILASLLALALILAVCIAVNS

SEQ ID NO: 11 - protein, 21 aa, Artificial sequence
  Synthetic construct
VVVACMSIMALLLLLLLLLLY

SEQ ID NO: 12 - protein, 22 aa, Artificial sequence
  Synthetic construct
VPVLCLLAIIFILTAALSYVLC

SEQ ID NO: 13 - protein, 28 aa, Artificial sequence
  Synthetic construct
AVGLLAFLGLLFCLGVAMFTYQSLQGCP

SEQ ID NO: 14 - protein, 25 aa, Artificial sequence
  Synthetic construct
LLVIIVVTVGVITVLVVVIVAVICT

SEQ ID NO: 15 - protein, 24 aa, Artificial sequence
  Synthetic construct
VAVCAGVILVLMLLLGCAAVVVCV

SEQ ID NO: 16 - protein, 26 aa, Artificial sequence
  Synthetic construct
LGPAAIALMILAFLLLLLVPLLLLMC

SEQ ID NO: 17 - protein, 22 aa, Artificial sequence
  Synthetic construct
IIIGALCVAFILMLIILIVGIC

SEQ ID NO: 18 - protein, 26 aa, Artificial sequence
  Synthetic construct
YLHTVIPAVVVAAILLIAGIIAMICY

SEQ ID NO: 19 - protein, 22 aa, Artificial sequence
  Synthetic construct
ILGILGGILALLILILLLLLFL

SEQ ID NO: 20 - protein, 24 aa, Artificial sequence
  Synthetic construct
AGVIAVIVVVVIAVVAGIVVLVIS

SEQ ID NO: 21 - protein, 23 aa, Artificial sequence
  Synthetic construct
VLLVSVSGSVVLVVILIAAFVIS

SEQ ID NO: 22 - protein, 23 aa, Artificial sequence
  Synthetic construct
IIGSSAAGLVFLIAVVVIAIVCN

SEQ ID NO: 23 - protein, 26 aa, Artificial sequence
  Synthetic construct
VALFAAVGAGCVIFLLIIIFLTVLLL

SEQ ID NO: 24 - protein, 21 aa, Artificial sequence
  Synthetic construct
GIASGCIIFIVIIITLVVLLL

SEQ ID NO: 25 - protein, 24 aa, Artificial sequence
  Synthetic construct
LIAAGVIGGLFILVIVGLTFAVYV

SEQ ID NO: 26 - protein, 24 aa, Artificial sequence
  Synthetic construct
FPWLLIIIFGIFGLTVMLFVFLFS

SEQ ID NO: 27 - protein, 24 aa, Artificial sequence
  Synthetic construct
VGIIAGLVLFGAVITGAVVAAVMW

SEQ ID NO: 28 - protein, 21 aa, Artificial sequence
  Synthetic construct
IGGIITVFLIALVLTSTIVTL

SEQ ID NO: 29 - protein, 23 aa, Artificial sequence
  Synthetic construct
LIIALPVAVLLIVGGLVIMLYVF

SEQ ID NO: 30 - protein, 20 aa, Artificial sequence
  Synthetic construct
HMIGICVTLTVIIVCSVFIY

SEQ ID NO: 31 - protein, 26 aa, Artificial sequence
  Synthetic construct
ASSTFSWGIVLAPLSLAFLVLGGIWM

SEQ ID NO: 32 - protein, 21 aa, Artificial sequence
  Synthetic construct
TFLVAGGSLAFGTLLCIAIVL

SEQ ID NO: 33 - protein, 23 aa, Artificial sequence
  Synthetic construct
IIIGPLIFVFLFSVVIGSIYLFL

SEQ ID NO: 34 - protein, 25 aa, Artificial sequence
  Synthetic construct
MATIILSTFLLIGWVAFIITYPLSM

SEQ ID NO: 35 - protein, 21 aa, Artificial sequence
  Synthetic construct
QDLLAASLTAVLLGGWILFVM

SEQ ID NO: 36 - protein, 26 aa, Artificial sequence
  Synthetic construct
GLLVPVLCGAFSVLWLACVVLCVWWT

SEQ ID NO: 37 - protein, 23 aa, Artificial sequence
  Synthetic construct
ALYVLMVLGFFGFFTLGIMLSYI

SEQ ID NO: 38 - protein, 24 aa, Artificial sequence
  Synthetic construct
VILYLMVMIGMFSFIIVAILVSTV

SEQ ID NO: 39 - protein, 23 aa, Artificial sequence
  Synthetic construct
YMYILFVMFLFAVTVGSLILGYT

SEQ ID NO: 40 - protein, 23 aa, Artificial sequence
  Synthetic construct
YFYILVVMSFYGIFLIGIMLGYM

SEQ ID NO: 41 - protein, 23 aa, Artificial sequence
  Synthetic construct
LLAFIAFLFFASIISLSLIFYYS

SEQ ID NO: 42 - protein, 21 aa, Artificial sequence
  Synthetic construct
FIGLMCAIALLTLLLLTVCFV

SEQ ID NO: 43 - protein, 21 aa, Artificial sequence
  Synthetic construct
VTGPVLAVILIILIVIAILLF

SEQ ID NO: 44 - protein, 25 aa, Artificial sequence
  Synthetic construct
HIASILIPLLLLLLLVLVAGVVFWY

SEQ ID NO: 45 - protein, 23 aa, Artificial sequence
  Synthetic construct
AIIVPLVLLVTLITTLVIGLVLC

SEQ ID NO: 46 - protein, 23 aa, Artificial sequence
  Synthetic construct
AVAVLLTILLIVVIGALAIAGFF

SEQ ID NO: 47 - protein, 25 aa, Artificial sequence
  Synthetic construct
TNTVGSVIGVIVTIFVSGTVYFICQ

SEQ ID NO: 48 - protein, 28 aa, Artificial sequence
  Synthetic construct
WGIALLVLVCVLVALAIVYLIALAVCQC

SEQ ID NO: 49 - protein, 22 aa, Artificial sequence
  Synthetic construct
IIAILLCIIILLILVLMFVVWM

SEQ ID NO: 50 - protein, 22 aa, Artificial sequence
  Synthetic construct
IMMYVLIVVLTIWLVAEMIYCY

SEQ ID NO: 51 - protein, 21 aa, Artificial sequence
  Synthetic construct
VIVGASVGGFLAVVILVLMVV

SEQ ID NO: 52 - protein, 22 aa, Artificial sequence
  Synthetic construct
IMMYILLVFLTLWLLIEMIYCY

SEQ ID NO: 53 - protein, 21 aa, Artificial sequence
  Synthetic construct
TLIILAVVGGVIGLLILILLI

SEQ ID NO: 54 - protein, 23 aa, Artificial sequence
  Synthetic construct
IIGGVAGSILLVLIVVGGIVVAL

SEQ ID NO: 55 - protein, 23 aa, Artificial sequence
  Synthetic construct
VLTITGICIALLVVGIMCVVAYC

SEQ ID NO: 56 - protein, 23 aa, Artificial sequence
  Synthetic construct
VLTITGICVALLVVGIVCVVAYC

SEQ ID NO: 57 - protein, 22 aa, Artificial sequence
  Synthetic construct
FMYVAAAAFVLLFFVGCGVLLS

SEQ ID NO: 58 - protein, 23 aa, Artificial sequence
  Synthetic construct
LLYLLAVAVVIILFIILLGVIMA

SEQ ID NO: 59 - protein, 22 aa, Artificial sequence
  Synthetic construct
LPLLVAGAVLLLVILVLGVMVA

SEQ ID NO: 60 - protein, 24 aa, Artificial sequence
  Synthetic construct
PVLCSPVAGVILLALGALLVLQLI

SEQ ID NO: 61 - protein, 23 aa, Artificial sequence
  Synthetic construct
SAVTGIVVGALLGAGLLMAFYFF

SEQ ID NO: 62 - protein, 22 aa, Artificial sequence
  Synthetic construct
IIPVYCALLATVILGLLAYVAF

SEQ ID NO: 63 - protein, 22 aa, Artificial sequence
  Synthetic construct
LIPVYCSILAAVVVGLVAYIAF

SEQ ID NO: 64 - protein, 24 aa, Artificial sequence
  Synthetic construct
VPVVLITTLLVIPVVVLLAIAIFI

SEQ ID NO: 65 - protein, 24 aa, Artificial sequence
  Synthetic construct
GLIIGILILVLIVATAILVTVYMY

SEQ ID NO: 66 - protein, 24 aa, Artificial sequence
  Synthetic construct
IILAASLSSVASWLALSCLVCCWL

SEQ ID NO: 67 - protein, 24 aa, Artificial sequence
  Synthetic construct
VYLIIAICAVSSLLVLTLLLYTAL

SEQ ID NO: 68 - protein, 22 aa, Artificial sequence
  Synthetic construct
LLLSLILVSVGFVVTVFGVIIF

SEQ ID NO: 69 - protein, 26 aa, Artificial sequence
  Synthetic construct
AVIPLVIVSALTFICLVVLVGILIYW

SEQ ID NO: 70 - protein, 22 aa, Artificial sequence
  Synthetic construct
LALGILGGLGTAALLIGVILWQ

SEQ ID NO: 71 - protein, 22 aa, Artificial sequence
  Synthetic construct
IAGISAGILVFILLLLVVILIV

SEQ ID NO: 72 - protein, 22 aa, Artificial sequence
  Synthetic construct
IAGVIAGILLFVIIFLGVVLVM

SEQ ID NO: 73 - protein, 22 aa, Artificial sequence
  Synthetic construct
AFIAGIGAACWIILMVFSIWLY

SEQ ID NO: 74 - protein, 21 aa, Artificial sequence
  Synthetic construct
AMLMLLSVVFVGLAVFLIYKF

SEQ ID NO: 75 - protein, 21 aa, Artificial sequence
  Synthetic construct
GSAMLMLLSVVFVGLAVFVIY

SEQ ID NO: 76 - protein, 23 aa, Artificial sequence
  Synthetic construct
AVVVPILFLILLSLGVGFAILYT

SEQ ID NO: 77 - protein, 23 aa, Artificial sequence
  Synthetic construct
SVPIILAIVGLMLVTVVAGVLIV

SEQ ID NO: 78 - protein, 22 aa, Artificial sequence
  Synthetic construct
GVIAGGLVGLIFAVCLVGFMLY

SEQ ID NO: 79 - protein, 25 aa, Artificial sequence
  Synthetic construct
VLAAVIAGGVIGFLFAIFLILLLVY

SEQ ID NO: 80 - protein, 22 aa, Artificial sequence
  Synthetic construct
AVIVGGVVGALFAAFLVTLLIY

SEQ ID NO: 81 - protein, 27 aa, Artificial sequence
  Synthetic construct
QLILAVVGSVSATCLTILAALLTLVCI

SEQ ID NO: 82 - protein, 24 aa, Artificial sequence
  Synthetic construct
WLLGAAMVGAVLTALLAGLVSLLC

SEQ ID NO: 83 - protein, 24 aa, Artificial sequence
  Synthetic construct
IIAIAVVGALLLVALIFGTASYLI

SEQ ID NO: 84 - protein, 23 aa, Artificial sequence
  Synthetic construct
LLVVMGTLVALVGLFVLLAFLQY

SEQ ID NO: 85 - protein, 24 aa, Artificial sequence
  Synthetic construct
LLIAPALAAVLLAALAAVGAAYCV

SEQ ID NO: 86 - protein, 21 aa, Artificial sequence
  Synthetic construct
AVVAILLCILTITVITLLIFL

SEQ ID NO: 87 - protein, 22 aa, Artificial sequence
  Synthetic construct
LITLTCTCVAATLFWLLLTLFI

SEQ ID NO: 88 - protein, 22 aa, Artificial sequence
  Synthetic construct
AAWAILPLLLLVMAAVGGYLMW

SEQ ID NO: 89 - protein, 24 aa, Artificial sequence
  Synthetic construct
MYPMFLVLLALAVLALAAVGVVVS

SEQ ID NO: 90 - protein, 22 aa, Artificial sequence
  Synthetic construct
VITGIILVIIALAFFGMVLSTQ

SEQ ID NO: 91 - protein, 22 aa, Artificial sequence
  Synthetic construct
PMLSMLVIPLLIIFVFMMVIVN

SEQ ID NO: 92 - protein, 22 aa, Artificial sequence
  Synthetic construct
PMYVVVAALVLLAFIGVGVLVS

SEQ ID NO: 93 - protein, 23 aa, Artificial sequence
  Synthetic construct
LITLTVGFGILIFSLIVTYCINA

SEQ ID NO: 94 - protein, 23 aa, Artificial sequence
  Synthetic construct
ALWPFLGIVAEVLVLVTIIFIYE

SEQ ID NO: 95 - protein, 273 aa, Artificial sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 96 - protein, 5 aa, Artificial sequence
  Synthetic construct
RKRRR

SEQ ID NO: 97 - protein, 309 aa, Artificial sequence
  Synthetic construct
ILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLH

SEQ ID NO: 98 - protein, 32 aa, Artificial sequence
  Synthetic construct
ILDYSFGGGAGRDIPPPLIEETVEPPPPAQLH

SEQ ID NO: 99 - protein, 27 aa, Artificial sequence
  Synthetic construct
TTTPAPRPPTPAPTIASQPLSLRPEAC

SEQ ID NO: 100 - protein, 21 aa, Artificial sequence
  Synthetic construct
MALPVTALLLPLALLLHAARP

SEQ ID NO: 101 - protein, 10 aa, Artificial sequence
  Synthetic construct
EQKLISEEDL

SEQ ID NO: 102 - DNA, 20 bp, Artificial sequence
  Synthetic construct
ggagcactgtcctccgaacg

SEQ ID NO: 103 - protein, 4 aa, Artificial sequence
  Synthetic construct
  MISC_FEATURE (2)..(3): X can be any amino acid residue
  MISC_FEATURE (4)..(4): X represents a hydrophobic residue such as Leu, Ile, Val, Phe, Trp, Tyr, Val, Met, and Pro
PXXX

SEQ ID NO: 104 - protein, 5 aa, Artificial sequence
  Synthetic construct
  MISC_FEATURE (2)..(3): X can be any amino acid residue
  MISC_FEATURE (4)..(4): X represents a hydrophobic residue such as Leu, Ile, Val, Phe, Trp, Tyr, Val, Met, and Pro
  MISC_FEATURE (5)..(5): X is either Ser or Thr
PXXXX

SEQ ID NO: 105 - protein, 6 aa, Artificial sequence
  Synthetic construct
  MISC_FEATURE (2)..(2): X is either Leu or Gln
PXGMTS

SEQ ID NO: 106 - protein, 5 aa, Artificial sequence
  Synthetic construct
  MISC_FEATURE (2)..(2): X is either Leu or Gln
PXGMT

SEQ ID NO: 107 - protein, 3 aa, Artificial sequence
  Synthetic construct
VGR

SEQ ID NO: 108 - protein, 7 aa, Artificial sequence
  Synthetic construct
ENLYTQS

SEQ ID NO: 109 - protein, 5 aa, Artificial sequence
  Synthetic construct
DDDDK

SEQ ID NO: 110 - protein, 4 aa, Artificial sequence
  Synthetic construct
LVPR

SEQ ID NO: 111 - protein, 556 aa, Artificial Sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 112 - protein, 538 aa, Artificial Sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 113 - protein, 550 aa, Artificial Sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 114 - protein, 538 aa, Artificial Sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSESKYGPPCPPCPFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 115 - protein, 697 aa, Artificial Sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 116 - protein, 539 aa, Artificial Sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 117 - protein, 542 aa, Artificial Sequence
  Synthetic construct
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVLTTTPAPRPPTPAPTIASQPLSLRPEACFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 118 - protein, 525 aa, Artificial Sequence
  Synthetic construct
METDTLLLWVLLLWVPGSTGDMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKTTTPAPRPPTPAPTIASQPLSLRPEACFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 119 - protein, 537 aa, Artificial Sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEAFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 120 - protein, 529 aa, Artificial Sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSRPAAGGAVHTRGLDFACDFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 121 - protein, 530 aa, Artificial Sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSCRPAAGGAVHTRGLDFACDFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 122 - protein, 543 aa, Artificial Sequence
  Synthetic construct
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVLTTTPAPRPPTPAPTIASQPLSLRPEACFMYVAAAAFVLLFFVGCGVLLSKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 123 - protein, 542 aa, Artificial Sequence
  Synthetic construct
MALPVTALLLPLALLLHAARPEQKLISEEDLQVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKTTTPAPRPPTPAPTIASQPLSLRPEACFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 124 - protein, 543 aa, Artificial Sequence
  Synthetic construct
MALPVTALLLPLALLLHAARPEQKLISEEDLQVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKTTTPAPRPPTPAPTIASQPLSLRPEACFMYVAAAAFVLLFFVGCGVLLSKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 125 - protein, 542 aa, Artificial Sequence
  Synthetic construct
MALPVTALLLPLALLLHAARPEQKLISEEDLDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGSTTTPAPRPPTPAPTIASQPLSLRPEACFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 126 - protein, 543 aa, Artificial Sequence
  Synthetic construct
MALPVTALLLPLALLLHAARPEQKLISEEDLDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGSTTTPAPRPPTPAPTIASQPLSLRPEACFMYVAAAAFVLLFFVGCGVLLSKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 127 - protein, 542 aa, Artificial Sequence
  Synthetic construct
MALPVTALLLPLALLLHAARPEQKLISEEDLDIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGSTTTPAPRPPTPAPTIASQPLSLRPEACFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 128 - protein, 543 aa, Artificial Sequence
  Synthetic construct
MALPVTALLLPLALLLHAARPEQKLISEEDLDIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGSTTTPAPRPPTPAPTIASQPLSLRPEACFMYVAAAAFVLLFFVGCGVLLSKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 129 - protein, 416 aa, Artificial Sequence
  Synthetic construct
MALPVTALLLPLALLLHAARPDYKDDDDKEVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSSTTTPAPRPPTPAPTIASQPLSLRPEACFMYVAAAAFVLLFFVGCGVLLSKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 130 - protein, 426 aa, Artificial Sequence
  Synthetic construct
MALPVTALLLPLALLLHAARPDYKDDDDKEVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSSFVPVFLPAKPTTTPAPRPPTPAPTIASQPLSLRPEACFMYVAAAAFVLLFFVGCGVLLSKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 131 - protein, 543 aa, Artificial Sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACATVVIVVCVSFLVFMIILGVFRIRAAHRRTMRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 132 - protein, 541 aa, Artificial Sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACIATVVIIISVCMLVFVVAMGVYRVRIAHQHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 133 - protein, 537 aa, Artificial Sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACATVVIVVCVSFLVFMIILGVFRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 134 - protein, 538 aa, Artificial Sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACIATVVIIISVCMLVFVVAMGVYRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 135 - protein, 11 aa, Artificial Sequence
  Synthetic construct
RIRAAHRRTMR

SEQ ID NO: 136 - protein, 8 aa, Artificial Sequence
  Synthetic construct
RVRIAHQH

SEQ ID NO: 137 - protein, 6 aa, Artificial Sequence
  Synthetic construct
KRKRKH

SEQ ID NO: 138 - protein, 45 aa, Artificial Sequence
  Synthetic construct
TTTPAPRPPTPAPTIASQPLSLRPEACRPAAGGAVHTRGLDFACD

SEQ ID NO: 139 - protein, 39 aa, Artificial Sequence
  Synthetic construct
IEVMYPPPYLDNEKSNGTIIHVKGKHLCPSPLFPGPSKP

SEQ ID NO: 140 - protein, 27 aa, Artificial Sequence
  Synthetic construct
GGGGSGGGGSGGGGSESKYGPPCPPCP

SEQ ID NO: 141 - protein, 186 aa, Artificial Sequence [sequence omitted]

SEQ ID NO: 142 - protein, 26 aa, Artificial Sequence
  Synthetic construct
TTTPAPRPPTPAPTIASQPLSLRPEA

SEQ ID NO: 143 - protein, 18 aa, Artificial Sequence
  Synthetic construct
RPAAGGAVHTRGLDFACD

SEQ ID NO: 144 - protein, 19 aa, Artificial Sequence
  Synthetic construct
CRPAAGGAVHTRGLDFACD

SEQ ID NO: 145 - protein, 37 aa, Artificial Sequence
  Synthetic construct
FVPVFLPAKPTTTPAPRPPTPAPTIASQPLSLRPEAC

SEQ ID NO: 146 - protein, 26 aa, Artificial Sequence
  Synthetic construct
TTTPAPRPPTPAPTIASQPLSLRPEA

SEQ ID NO: 147 - protein, 26 aa, Artificial Sequence
  Synthetic construct
TTTPAPRPPTPAPTIASQPLSLRPEA

SEQ ID NO: 148 - protein, 26 aa, Artificial Sequence
  Synthetic construct
TTTPAPRPPTPAPTIASQPLSLRPEA

(19) United States
(12) Patent Application Publication
(10) Pub. No.: US 2022/0348677 A1
(43) Pub. Date: Nov. 3, 2022

(54) RECEPTORS WITH HETEROLOGOUS TRANSMEMBRANE DOMAIN

(71) Applicant: The Regents of the University of California, Oakland, CA (US)

(72) Inventors: Kole T. ROYBAL, Oakland, CA (US); Raymond LIU, Oakland, CA (US); Iowis ZHU, Oakland, CA (US)

(21) Appl. No.: 17/763,116

(22) PCT Filed: Sep. 23, 2020
(86) PCT No.: PCT/US2020/052320

§ 371 Date: Mar. 23, 2022

Related U.S. Application Data
(60) Provisional application No. 62/905,258, filed on Sep. 24, 2019.

(51) Int. Cl. C07K 16/28; C07K 14/47

(52) U.S. Cl. CPC C07K 16/2896; C07K 14/4705; C07K 2317/622; C07K 2319/03; C07K 2319/035; C07K 2319/71

(57) ABSTRACT

The present disclosure generally relates to, among other things, a new class of receptors engineered to modulate transcriptional regulation in a ligand-dependent manner. In particular, the new receptors contain a heterologous transmembrane domain comprising at least one γ-secretase site. The disclosure also provides compositions and methods useful for producing such receptors, nucleic acids encoding same, host cells genetically modified with the nucleic acids, as well as methods for modulating an activity of a cell and/or for the treatment of various health conditions or diseases, such as cancers.

### CROSS-REFERENCE TO RELATED APPLICATIONS

This application claims priority to U.S. Provisional Patent Application No. 62/905,258, filed Sep. 24, 2019, the disclosure of which is incorporated by reference herein in its entirety, including any drawings.

### STATEMENT REGARDING FEDERALLY SPONSORED R&D

This invention was made with government support under grant no. OD025751 awarded by The National Institutes of Health. The government has certain rights in the invention.

### INCORPORATION OF THE SEQUENCE LISTING

This application contains a Sequence Listing which is hereby incorporated by reference in its entirety. The accompanying Sequence Listing text file, named “048536_658001WO_Sequence_Listing_ST25.txt,” was created on Sep. 23, 2020 and is 156 KB.

### FIELD

The present disclosure relates generally to new synthetic cellular receptors that bind cell-surface ligands and have selectable specificities and activities. The disclosure also provides compositions and methods useful for producing such receptors, nucleic acids encoding same, host cells genetically modified with the nucleic acids, as well as methods for modulating an activity of a cell and/or for the treatment of various health conditions or diseases, such as cancers.

### BACKGROUND

An important problem which limits the development of engineered cell therapies in humans is the ability to regulate therapeutic gene expression and engineered cell activity. For example, the first generation of chimeric antigen receptor T cells (CAR-T) lack the ability to modulate or turn off CAR-T activity when needed; other problems include off-target activity and off-tumor/on-target activity (i.e., wherein the CAR-T target antigen is also found on normal cells outside the tumor). One possible solution to these problems is to use a synthetic receptor that is capable of modifying gene expression and/or cellular behavior.

Notch receptors are Type 1 transmembrane proteins that mediate cell-cell signaling and play a central role in development and other aspects of cell-to-cell communication, e.g. communication between two contacting cells, in which one contacting cell is a “receiver” cell and the other contacting cell is a “sender” cell. Notch receptors expressed in a receiver cell recognize their ligands (e.g., the delta/serrate/lag, or “DSL” family of proteins), expressed on a sending cell. The engagement of Notch and the DSL ligand on these contacting cells leads to two-step proteolysis of the Notch receptor that ultimately causes the release of the intracellular portion of the receptor from the membrane into the cytoplasm. Notch has a metalloprotease cleavage site (denoted “S2”), which is normally protected from cleavage by the Notch negative regulatory region (NRR), which contains three LIN-12-Notch repeat (LNR) modules and a heterodimerization domain (HD) of the Notch extracellular subunit (NEC). Positioned C-terminal of the HD domain is the transmembrane domain (TMD). It contains the S3 cleavage site, which is a substrate for regulated intramembrane proteolysis by the γ-secretase complex (γSec). S3 proteolysis results in the release of the Notch intracellular domain. This event will occur only after the rate-limiting S2 cleavage has taken place, making S3 accessible to γSec.

It is believed that this proteolysis is regulated by the force exerted by the sending cell: the DSL ligand pulls on the Notch receptor, changing the conformation of the (NRR), and exposing the S2 metalloprotease site. This is cleaved by a constitutively active protease, which releases the extracellular binding portion and negative regulatory region of the receptor. Release of the ligand binding portion of the receptor in turn exposes other intramembrane cleavage site(s) (e.g., S3 sites), which are cleaved by γ-secretase within the cell membrane and release the nuclear homing intracellular domain (ICD). W. R. Gordon et al., Dev Cell (2015) 33:729-36. This released domain alters receiver cell behavior by functioning as a transcriptional regulator. Thus, cleavage of the Notch transmembrane domain is an essential step in the Notch signaling pathway, which is involved in and required for a variety of cellular functions during development. In addition, the prevention of this cleavage process was previously reported to cause significant dysregulation and disease. Moreover, recent reports that inhibition of γ-secretase cleavage of the Notch TMD can cause toxicity have accelerated efforts to prevent or treat Alzheimer's disease by inhibiting γ-secretase cleavage of the amyloid precursor protein.

Receptors, whether native or synthetic, have varying characteristics, such as “noise” (i.e., the baseline level of expression induced in the absence of the intended ligand), and signal or sensitivity (the amount of expression induced by binding of the intended ligand). Generally, the signaling through Notch and the existing first-generation synthetic derivatives of Notch receptors, which are often referred to as “SynNotch” correlates with ligand binding, but it is difficult to adjust the sensitivity and response of the receptor, and more tools are needed in order to provide synthetic receptors with a wider range of more easily regulatable characteristics.

### SUMMARY

The present disclosure describes synthetic receptors having a heterologous transmembrane domain (TMD). Surprisingly, altering this domain can dramatically affect the signal characteristics of the receptor, such as the degree of receptor expression, the signal level from ligand-induced activation, and the signal level in the absence of ligand. Provided herein are synthetic chimeric receptors that exhibit a range of signal characteristics mediated by inclusion of a heterologous TMD. These receptors provide a range of sensitivity, including a receptor that is sensitive to the degree of T-cell activation when it is expressed in an activated T cell. Some embodiments, when expressed in a T-cell, exhibit higher ligand-induced signal levels when the T-cell is activated, as compared to the ligand-induced signal level when the T-cell is not activated.

The present disclosure provides, among other things, novel chimeric receptors containing a heterologous transmembrane domain comprising at least one γ-secretase site. Since cleavage of TMD is an essential step in the Notch signaling pathway which is involved in and is required for a variety of cellular functions during development, it is believed that the modulation of TMD cleavage facilitates the optimization and/or improvement of the activity of the chimeric receptors disclosed herein, which in turn can be particularly useful in modulating cell activity and/or in treating health conditions, e.g., diseases.

In one aspect, provided herein are chimeric polypeptides including, from N-terminus to C-terminus: (a) an extracellular binding domain having a binding affinity for a selected ligand; (b) a linking sequence having: (i) at least about 80% sequence identity to a Notch JMD; (ii) at least about 80% sequence identity to a Notch JMD wherein the LIN-12-Notch repeat (LNR) and/or a heterodimerization domain (HD) of a Notch receptor has been deleted; (iii) at least about 80% sequence identity to a polypeptide hinge domain; (iv) at least about 80% sequence identity to a ROBO1 juxtamembrane domain (JMD) including at least one fibronectin (Fn) repeat; or (v) a polypeptide having about 2 to about 40 amino acids; (c) a transmembrane domain (TMD) having at least about 80% sequence identity to the transmembrane domain of a Type 1 transmembrane receptor and comprising one or more ligand-inducible proteolytic cleavage sites; and (d) an intracellular domain (ICD) comprising a transcriptional regulator, wherein binding of the selected ligand to the extracellular binding domain induces cleavage at the ligand-inducible proteolytic cleavage site between the transcriptional regulator and the linking sequence, and wherein (i) when the linking sequence has at least about 80% sequence identity to a Notch JMD or a Notch JMD wherein the LNR and/or a HD of a Notch receptor has been deleted, the TMD is heterologous to the linking sequence, and (ii) when the linking sequence does not have at least about 80% sequence identity to a Notch JMD or a Notch JMD wherein the LNR and/or a HD of a Notch receptor has been deleted, the transmembrane domain is not a Notch1 TMD.

Non-limiting exemplary embodiments of the chimeric polypeptides provided herein include one or more of the following features: in some embodiments, the chimeric polypeptide further includes a stop-transfer sequence (STS) between the TMD and the ICD; in some embodiments, the TMD comprises a polypeptide sequence having at least 80% sequence identity to a transmembrane domain from a Type 1 transmembrane receptor and comprises a γ-secretase cleavage site; in some embodiments, the TMD comprises a polypeptide sequence having at least 90% sequence identity to a transmembrane domain from a Type I transmembrane receptor and comprises a γ-secretase cleavage site; in some embodiments, the TMD comprises a polypeptide sequence having at least 95% sequence identity to a transmembrane domain from a Type I transmembrane receptor and comprises a γ-secretase cleavage site.

In some embodiments, the extracellular domain includes an antigen-binding moiety capable of binding to a ligand on the surface of a cell. In some embodiments, the cell is a pathogen. In some embodiments, the ligand includes a protein or a carbohydrate. In some embodiments, the ligand is a cluster of differentiation (CD) marker. In some embodiments, the CD marker is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, BCMA (CD269), ALPPL2, GFP, eGFP, and SIRPα.

In another aspect, provided herein are nucleic acids comprising a nucleotide sequence encoding a chimeric polypeptide as disclosed herein. In some embodiments, the nucleotide sequence is incorporated into an expression cassette or an expression vector.

In another aspect, provided herein are recombinant cells including (a) a chimeric polypeptide as disclosed herein and/or (b) a recombinant nucleic acid as disclosed herein. In another aspect, further provided herein are cell cultures including at least one recombinant cell as disclosed herein and a culture medium.

In another aspect, provided herein are pharmaceutical compositions including a pharmaceutically acceptable carrier and one or more of the following: (a) a recombinant nucleic acid as disclosed herein, or (b) a recombinant cell as disclosed herein. In some embodiments, the disclosed pharmaceutical composition includes a recombinant nucleic acid as disclosed herein and a pharmaceutically acceptable carrier. In some embodiments, the recombinant nucleic acid is encapsulated in a viral capsid or a lipid nanoparticle.

In another aspect, provided herein are methods for modulating an activity of a cell, including: (a) providing a recombinant cell of the disclosure, and (b) contacting it with a selected ligand, wherein binding of the selected ligand to the extracellular binding domain induces cleavage of a ligand-inducible proteolytic cleavage site and releases the transcriptional regulator, wherein the released transcriptional regulator modulates an activity of the recombinant cell. Another aspect relates to methods for inhibiting an activity of a target cell in an individual, including administering to the individual an effective number of the recombinant cell of the disclosure, wherein the recombinant cell inhibits an activity of the target cell in the individual.

In another aspect, provided herein are methods for treating a health condition (e.g., disease) in an individual, the methods comprising a step of administering to the individual an effective number of the recombinant cell of the disclosure, wherein the recombinant cell treats the health condition in the individual.

In another aspect, provided herein are systems for modulating an activity of a cell, modulating an activity of a target cell, or treating a health condition (e.g., disease) in an individual in need thereof, wherein the system includes one or more of: a chimeric polypeptide of the disclosure; a polynucleotide of the disclosure; a recombinant cell of the disclosure; or a pharmaceutical composition of the disclosure.

In another aspect, provided herein are methods for making a recombinant cell of the disclosure, including: (a) providing a cell capable of protein expression; and (b) contacting the provided cell with a recombinant nucleic acid of the disclosure. In some embodiments, the cell is obtained by leukapheresis performed on a sample obtained from a human subject or patient, and the cell is contacted ex vivo. In some embodiments, the recombinant nucleic acid is encapsulated in a viral capsid or a lipid nanoparticle.

In another aspect, provided herein is the use of one or more of: a chimeric polypeptide of the disclosure, a polynucleotide of the disclosure, a recombinant cell of the disclosure, or a pharmaceutical composition of the disclosure, for the treatment of a health condition (e.g., disease). In some embodiments, the health condition is cancer.

In another aspect, provided herein is the use of one or more of: a chimeric polypeptide of the disclosure, a polynucleotide of the disclosure, a recombinant cell of the disclosure, or a pharmaceutical composition of the disclosure, in the manufacture of a medicament for the treatment of a health condition (e.g., disease).

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1D schematically illustrate differences between a SynNotch receptor and the chimeric polypeptides of the disclosure. FIG. 1A depicts the schematic structure of an existing synthetic Notch receptor (SynNotch). FIG. 1B depicts the schematic structure of an exemplary second-generation SynNotch receptor having a heterologous transmembrane domain (TMD). In this chimeric SynNotch receptor, the TMD is heterologous with respect to the adjacent intracellular domain (ICD) and the linking polypeptide. FIG. 1C depicts another exemplary second-generation synthetic Notch receptor as disclosed herein. In this chimeric Notch receptor (MiniNotch), the LNR domain and HD domain (which together form the Notch regulatory region, NRR) of the wild-type Notch polypeptide have been deleted, and the TMD is heterologous to the extracellular domain (linking polypeptide). FIG. 1D depicts another exemplary second-generation synthetic Notch receptor as disclosed herein. In this chimeric Notch receptor (Hinge-Notch) receptor, the Notch extracellular subunit of the wild-type Notch polypeptide has been deleted, and a hinge polypeptide sequence derived from CD8α hinge domain is inserted N-terminally to the TMD. In each of these exemplary chimeric Notch receptors, the extracellular binding domain comprises a single-chain antigen-binding fragment (scFv) having a binding affinity for a selected ligand, which in this example is the B-lymphocyte antigen CD19.

FIG. 2 depicts the experimental design of a screen for transmembrane domains that can be deployed in the chimeric polypeptides and receptors described herein. The screen was performed with transmembrane domains derived from 88 known human γ secretase target (Haapalaso, J Alzheimers Dis. 25(1):3-28, 2011) and Notch family members from model organisms. The blue fluorescent reporter gene (BFG) was placed under control of a promoter having 4 copies of GAL4 recognition site (i.e., GAL4 response elements).

FIG. 3 summarizes the results of a screen for TMDs with a higher ligand-induced activation activity. In these experiments, amino acid sequences corresponding to transmembrane domains from 88 known human γ secretase target (Haapalaso, 2011) and Notch family members from model organisms were incorporated into the receptors schematically depicted in FIG. 1. A reporter positive Jurkat T-cell line was transduced with a receptor construct. Receptor expression was measured using an AlexaFluor647-tagged anti-myc antibody (Cell Signaling). For receptor activation testing, 1×10⁵ Jurkat T-cells expressing anti-CD19 receptors were co-cultured with: no additions, 1×10⁵ K562 cells, or 1×10⁵ CD19+K562 cells for 24 hours. Transcriptional activation of an inducible BFP reporter gene was measured using a Fortessa X-50 (BD Biosciences). % BFP+ is plotted according to the plate map (one TMD per well).

FIG. 4 shows: (top) a map of the 96-well plate, showing the construct plasmid used in each well as described in FIG. 3; (middle) the percent receptor positive of transduced Jurkat cell population in each well, as measured by AF647-anti-myc antibody staining; and (bottom) the corrected activation, calculated by dividing the uncorrected percent activation (% BFP+) by the percent of Jurkats that were myc-positive.

FIG. 5 shows a heat map of miniNotch1 TMD Screen in Jurkat T-cell line. A reporter positive Jurkat T-cell line was transduced with the receptor constructs. Receptor expression was measured using an AlexaFluor647-tagged anti-myc antibody (Cell Signaling). For receptor activation testing, 1×10⁵ Jurkat T-cells expressing anti-CD19 receptors were co-cultured with: no additions, 1×10⁵ K562 cells, or 1×10⁵ CD19+K562 cells for 24 hours. Transcriptional activation of an inducible BFP reporter gene was measured using a Fortessa X-50 (BD Biosciences). % BFP+ was plotted according to the plate map (one TMD per well).

FIG. 6 shows: (top) a map of the 96-well plate, showing the construct plasmid used in each well as described in FIG. 5; (middle) the percent receptor positive of transduced Jurkat cell population in each well, as measured by AF647-anti-myc antibody staining; and (bottom) the corrected activation, calculated by dividing the uncorrected percent activation (% BFP+) by the percent of Jurkats that were myc-positive.

FIG. 7 depicts the experimental design to show that TMD regulates Notch activation. Jurkat T cells expressing a BFP reporter construct were transduced with lentiviral constructs containing Notch receptors with TMD variants. Jurkats were co-cultured 1:1 with control CD19(−) or CD19(+) K562 cells. BFP reporter gene activation was subsequently measured using a Fortessa X-50 (BD Biosciences). Signal to noise ratios from the MFIs of BFP+ cells under CD19+K562 versus K562 conditions are plotted against the change in MFI in the two conditions.

FIGS. 8A-8B schematically summarize the results from experiments for mutational analysis of the Notch1 transmembrane domain (TMD) in Hinge-Notch constructs. Variants with different alanine mutations in the TMD domain of the Hinge-Notch construct were prepared. Each amino acid residue from position 301 (F) through position 322 (S) in the TMD of Hinge-Notch were individually mutated to alanine. Primary human CD4+ T-cells were activated with anti-CD3/anti-CD28 Dynabeads and transduced with two lentiviral constructs, one expressing a TMD mutant variant, and the other containing a BFP transcriptional reporter. Cells containing both constructs were sorted for on Day 5 post initial T-cell stimulation and expanded further for activation testing. In FIG. 8A, the left panel shows relative expression of different receptors, measured by anti-myc-tag staining (y-axis), versus reporter construct marker expression (x-axis), while the right panel represents MFI quantitation of receptor expression of TMD mutant variants in double-positive cells. In FIG. 8B, T-cells expressing anti-CD19 receptors were co-cultured at a ratio of 1:1 with control CD19(−) or CD19(+) K562 cells. Transcriptional activation of an inducible BFP reporter gene was subsequently measured using a Fortessa X-50. The left panel shows flow panels of activation profiles. The right panel represents BFP % plotted as a line graph.

FIG. 9 schematically summarizes the results from experiments for mutational analysis for the transmembrane domain (TMD) and the STS domain in Hinge-Notch constructs. Four types of exemplary Hinge Notch receptors were using in this Example, all of which including an anti-CD19 scFv domain, a truncated CD8 Hinge domain, and a Gal4VP64 domain, plus different TMD domains (CLSTN1 TMD or CLSTN2 TMD) and different STS domains (CLSTN1 STS, CLSTN2 STS, or Notch1 STS). Primary human CD4+ T-cells were activated with anti-CD3/anti-CD28 Dynabeads (Gibco) and transduced with two lentiviral constructs, one expressing a hinge receptor with TMD/STS combination as indicated, and the other a transcriptional reporter with constitutively expressed anti-ALPPL2 CAR. Cells containing both constructs were sorted for on Day 5 post initial T-cell stimulation and expanded further for activation testing. For testing, 1×10⁵ double positive T-cells expressing receptors were co-cultured with: 1×10⁵ K562 cells (“−CAR” panels, blue), or 1×10⁵ CD19+K562 cells (“−CAR” panels, red). Similarly, 1×10⁵ double positive T-cells expressing receptors were tested in the presence of CAR activity by co-culture with 1×10⁵ ALPPL2+K562 cells (“+CAR” panels, blue), or 1×10⁵ ALPPL2+CD19+K562 cells (“+CAR” panels, red). Transcriptional activation of an inducible BFP reporter gene was subsequently measured using a Fortessa X-50 (BD Biosciences).

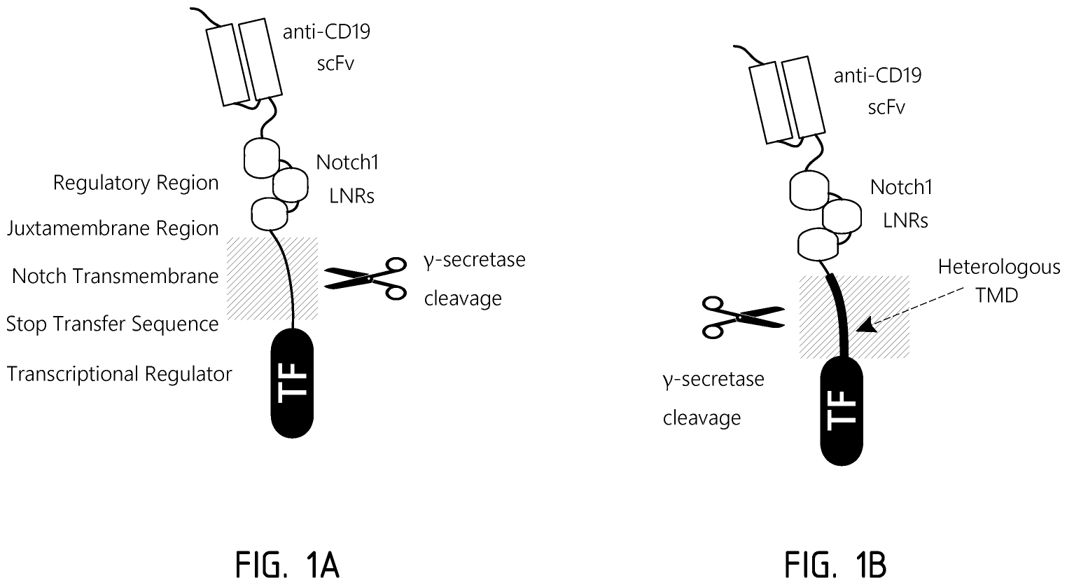

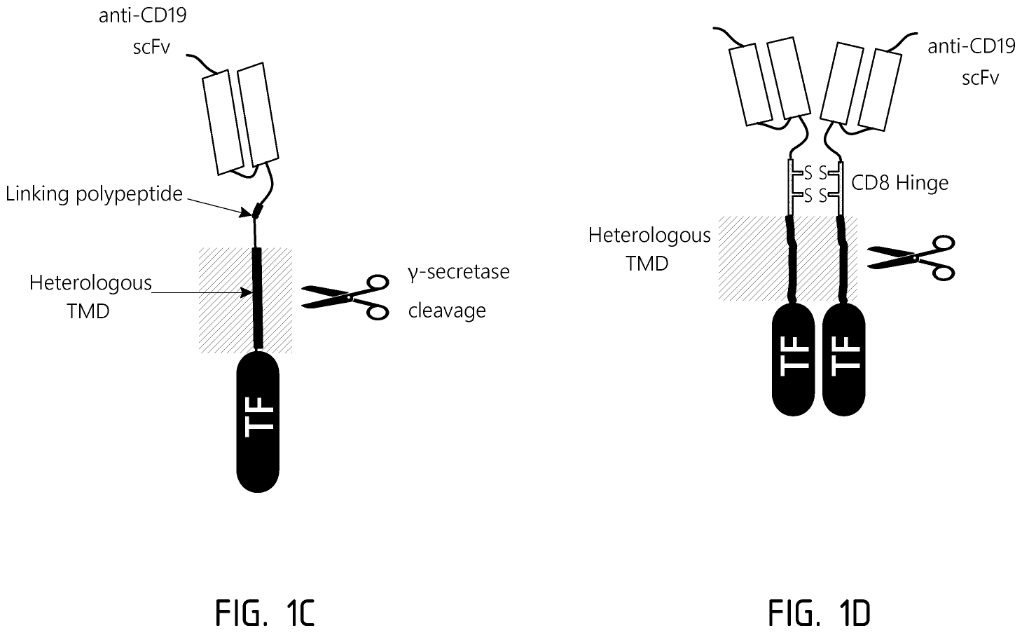

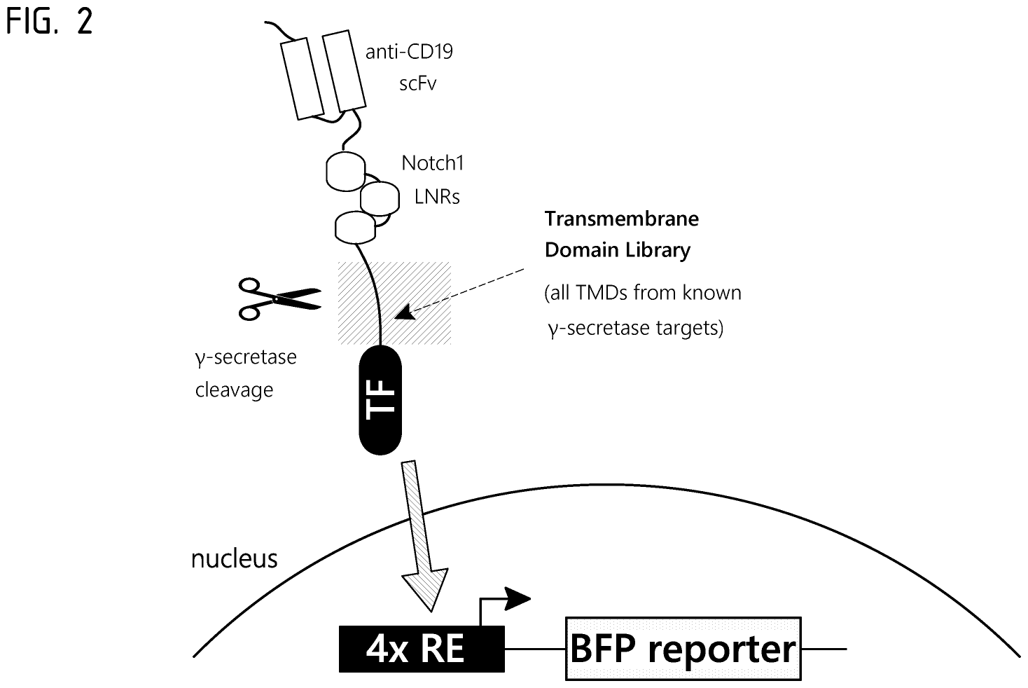

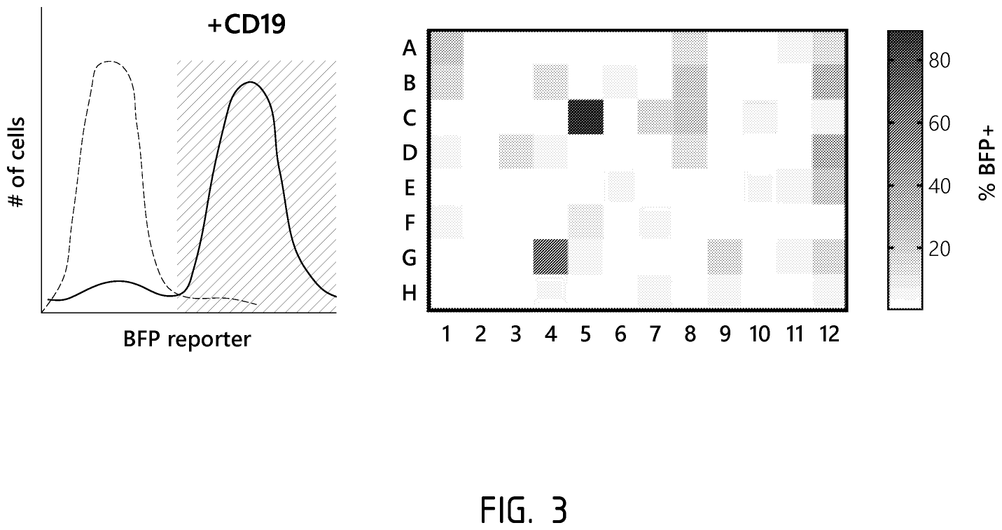

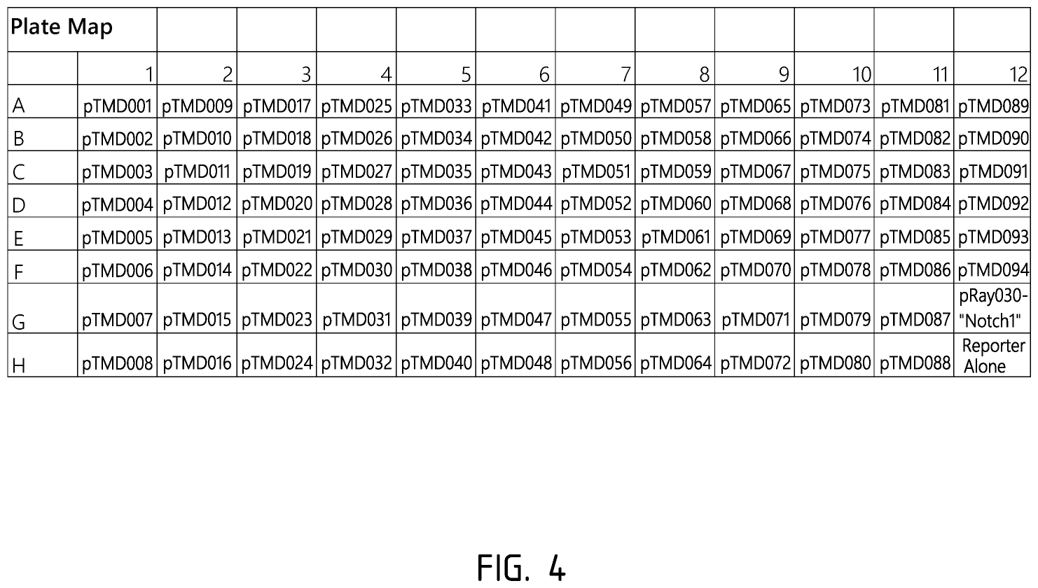

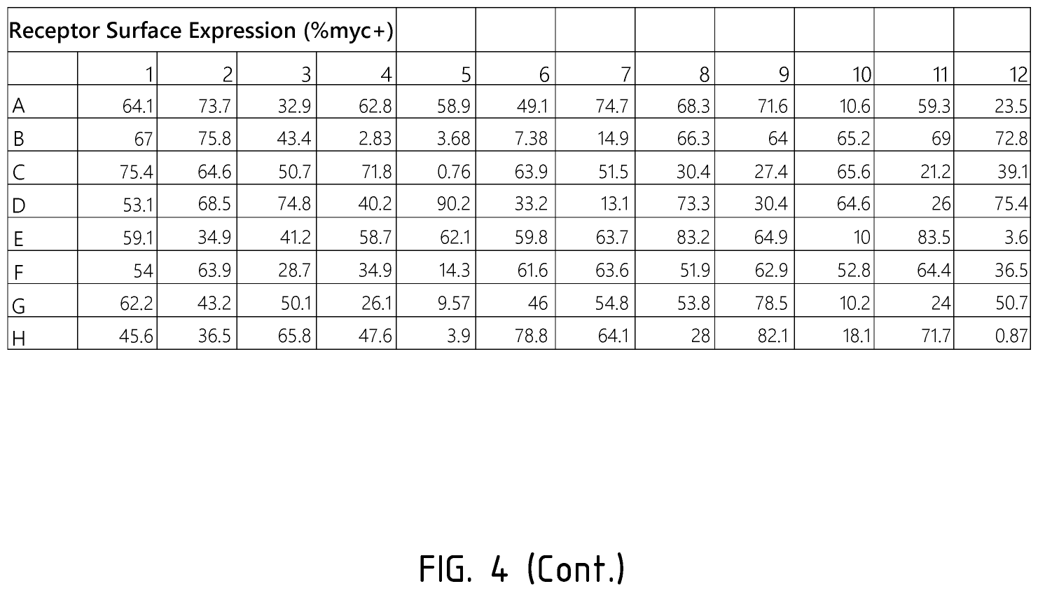

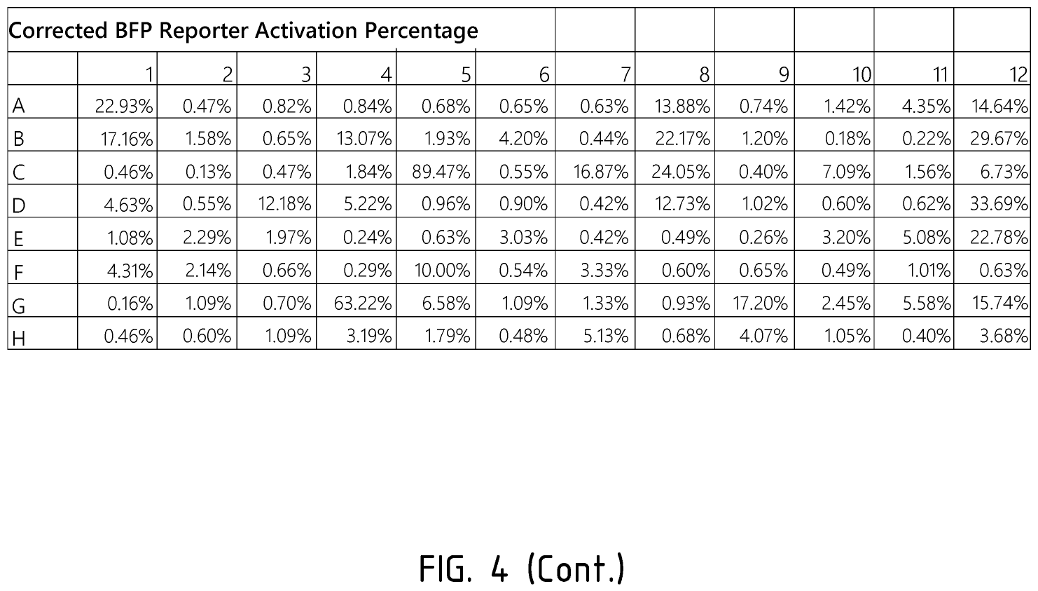

### DETAILED DESCRIPTION OF THE DISCLOSURE

The present disclosure generally relates to, among other things, novel chimeric Notch receptors containing a heterologous transmembrane domain having at least one γ-secretase site. These receptors do not occur in nature. As described below, the chimeric polypeptides of the disclosure can be synthetic polypeptides, or can be engineered, designed, or modified so as to provide desired and/or improved properties, e.g., modulating transcription. As described in greater detail below, cleavage of the TMD is an essential step in signaling mediated by Notch-type receptors. Without being bound to any particular theory, it is believed that modulation of TMD cleavage facilitates the optimization and/or improvement of the receptors disclosed herein, which in turn can be particularly useful in modulating cell activity, e.g., activating or inhibiting selected biosynthetic pathways and/or in treating health conditions or diseases. In some embodiments, the receptors disclosed herein bind a target cell-surface displayed ligand, which triggers proteolytic cleavage of the receptors and release of a transcriptional regulator that modulates a custom transcriptional program in the cell. The disclosure also provides compositions and methods useful for producing such receptors, nucleic acids encoding same, host cells genetically modified with the nucleic acids, as well as methods for modulating an activity of a cell and/or for the treatment of various health conditions or diseases, such as cancers.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

The terms “administration” and “administering”, as used interchangeably herein, refer to the delivery of a composition or formulation by an administration route including, but not limited to, intravenous, intra-arterial, intracerebral, intrathecal, intramuscular, intraperitoneal, subcutaneous, intramuscular, and combinations thereof. The term includes, but is not limited to, administration by a medical professional and self-administration.

The terms “host cell” and “recombinant cell” are used interchangeably herein. It is understood that such terms, as well as “cell”, “cell culture”, “cell line”, refer not only to the particular subject cell or cell line but also to the progeny or potential progeny of such a cell or cell line, without regard to the number of transfers. It should be understood that not all progeny are exactly identical to the parental cell. This is because certain modifications may occur in succeeding generations due to either mutation (e.g., deliberate or inadvertent mutations) or environmental influences, such progeny may not, in fact, be identical to the parent cell, but are still included within the scope of the term as used herein, so long as the progeny retain the same functionality as that of the original cell or cell line.

The term “operably linked”, as used herein, denotes a physical or functional linkage between two or more elements, e.g., polypeptide sequences or polynucleotide sequences, which permits them to operate in their intended fashion.

The term “heterologous”, refers to nucleic acid sequences or amino acid sequences operably linked or otherwise joined to one another in a nucleic acid construct or chimeric polypeptide that are not operably linked or are not contiguous to each other in nature.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See, e.g., the NCBI web site at ncbi.nlm.nih.gov/BLAST. This definition also refers to, or may be applied to, the complement of a test sequence. This definition also includes sequences that have deletions and/or additions, as well as those that have substitutions. Sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res (1984) 12:387), BLASTP, BLASTN, FASTA (Atschul et al., J Mol Biol (1990) 215:403). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof

As used herein, and unless otherwise specified, a “therapeutically effective amount” of an agent is an amount sufficient to provide a therapeutic benefit in the treatment or management of the cancer, or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human individuals) and non-human animals. In some embodiments, a “subject” or “individual” can be a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, sheep, dogs, cows, chickens, amphibians, reptiles, and the like.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and so forth. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Notch Receptors

Notch receptors are highly conserved single pass transmembrane proteins essential to a wide spectrum of cellular systems, and their dysregulation has been linked to a number of developmental disorders and malignancies. Notch receptors normally communicate signals upon binding to surface-bound ligands expressed on adjacent cells. Notch signals rely on cell-cell contact. Evolutionary divergence of vertebrates and invertebrates has been accompanied by at least two rounds of gene duplication in the Notch lineage: flies possess a single Notch gene, worms two (GLP-1 and LIN-12), and mammals four (NOTCH1-4). Transduction of Notch signals relies on three key events: (i) ligand recognition; (ii) conformational exposure of the ligand-dependent cleavage site followed by cleavage and release of the nuclear-homing intracellular domain; and (iii) assembly of nuclear transcriptional activation complexes.

Canonical Notch signals are transduced by regulated intramembrane proteolysis. Notch receptors are normally maintained in a resting, proteolytically resistant conformation on the cell surface, but ligand binding initiates a proteolytic cascade that releases the ICD from the membrane. The critical, regulated cleavage step is effected by one or more ADAM metalloproteases, occurs at a site called S2 immediately external to the plasma membrane, and results in a truncated receptor. This truncated receptor remains membrane tethered until it is cleaved at one or more sites (called “S3”) by γ-secretase, a multiprotein enzyme complex.

After γ-secretase cleavage at S3, the ICD enters the nucleus, where it assembles a transcriptional activation complex that contains a DNA-binding transcription factor and engages additional coactivator proteins, such as p300, to recruit the basal transcription machinery and activate the expression of downstream target genes.

Notch receptors have a modular domain organization. The ectodomains of Notch receptors consist of a series of N-terminal epidermal growth factor (EGF)-like repeats that are responsible for ligand binding. O-linked glycosylation of these EGF repeats, including modification by O-fucose, Fringe, and Rumi glycosyltransferases, also modulates the activity of Notch receptors in response to different ligand subtypes in vertebrates and invertebrates.

The EGF repeats are followed by three LIN-12/Notch repeat (LNR) modules, which are unique to Notch receptors, and are widely reported to participate in preventing premature receptor activation. The heterodimerization (HD) domain of Notch1 is cleaved by furin, so that its N-terminal part terminates the extracellular subunit, and its C-terminal part constitutes the beginning of the TMD subunit, resulting in a heterodimer that remains bound together by non-covalent interactions. Following the extracellular region, the receptor has a transmembrane segment and an intracellular domain (ICD), which includes a transcriptional regulator. Additional information regarding Notch receptors and Notch-mediated cell signaling can be found in, for example, W. R. Gordon et al., Dev Cell (2015) 33:729-36 and W. R. Gordon et al., J. Cell Sci. (2008) 121:3109-19, both of which are hereby incorporated by reference.

### Compositions of the Disclosure

As described in greater detail below, the present disclosure provides a new class of chimeric polypeptide receptors engineered to modulate transcriptional regulation in a ligand-dependent manner having multiple advantages over existing synthetic Notch receptors. For example, replacing a TMD with a heterologous TMD provides new receptors having a variety of improved expression characteristics, thus expanding the palette of available receptors available to the cellular engineer. Further, in some embodiments of the chimeric polypeptide and receptors disclosed herein, by omitting the Notch/SynNotch regulatory regions, or the entire NEC subunit, polynucleotides encoding the chimeric receptors provided herein can be made smaller than SynNotch-encoding polynucleotides, which enables the use of vectors having more limited capacity but otherwise more desirable characteristics, or the inclusion of additional elements that would otherwise be excluded vector capacity-related size constraints.

One skilled in the art will understand that the chimeric polypeptide receptors disclosed herein facilitate amplified activation under certain specific cellular and environmental contexts. This type of feedback on the receptor activity is a new feature that can be exploited to enhance and tune the expression of therapeutic payloads by engineered cells. In addition, as described in further detail below, a number of the receptor variants disclosed herein are expressed at higher rates than existing SynNotch receptors.

In addition, as described in greater detail below, the chimeric polypeptide receptors disclosed herein have improved activity compared to existing SynNotch receptors and provide a more modular platform for engineering. Existing SynNotch receptors can be engineered with ligand-binding domains such scFvs and nanobodies, but it has been difficult to use natural extracellular domains from receptors/ligands on SynNotch receptors. In contrast, second-generation Notch receptors of the disclosure are amenable to substitution of the Notch ECD with other types of ligand binding domains—not just those derived from antibodies, thus expanding the landscape of targetable diseases and tissues.

As described in the Examples herein, chimeric polypeptide receptors have been tested and validated in primary human T cells. Without being bound to any particular theory, it is contemplated that these new receptors show similar performance in mouse models and in other mammalian cells. The receptors disclosed herein may be engineered into various immune cell types for enhanced discrimination and elimination of tumors, or in engineered cells for control of autoimmunity and tissue regeneration. Accordingly, engineered cells, such as immune cells engineered to express one of more of the chimeric receptors disclosed herein, are also within the scope of the disclosure.

### Chimeric Polypeptides

In one aspect, provided herein are a variety of novel, non-naturally occurring chimeric polypeptides engineered to modulate transcriptional regulation in a ligand-dependent manner. These new receptors comprise a heterologous TMD having at least one γ-secretase site. Since cleavage of the Notch transmembrane domain is an essential step in Notch receptor function, it is believed that the modulation of Notch TMD cleavage facilitates the optimization and/or improvement of the new chimeric receptors disclosed herein, which in turn is useful in modulating cell activity and/or in treating health conditions, e.g., diseases. In some embodiments, the receptors provided herein bind a target cell-surface displayed ligand, triggering proteolytic cleavage of the receptors and releasing a transcriptional regulator that modulates a custom transcriptional program in the cell.

In some embodiments, provided herein is a chimeric polypeptide including, from N-terminus to C-terminus: (a) an extracellular binding domain having a binding affinity for a selected ligand; (b) a linking sequence having: (i) at least about 80% sequence identity to a Notch JMD); (ii) at least about 80% sequence identity to a Notch JMD wherein the LNR and/or an HD of a Notch receptor has been deleted; (iii) at least about 80% sequence identity to a polypeptide hinge domain; (iv) at least about 80% sequence identity to a ROBO1 JMD including at least one fibronectin (Fn) repeat; or (v) a polypeptide having about 2 to about 40 amino acids; (c) a TMD having at least about 80% sequence identity to the transmembrane domain of a Type 1 transmembrane receptor and comprising one or more ligand-inducible proteolytic cleavage sites; and (d) an ICD comprising a transcriptional regulator, wherein binding of the selected ligand to the extracellular binding domain induces cleavage at the ligand-inducible proteolytic cleavage site between the transcriptional regulator and the linking sequence, and wherein (i) when the linking sequence has at least about 80% sequence identity to a Notch JMD or a Notch JMD wherein the LNR and/or an HD of a Notch receptor has been deleted, the transmembrane domain is heterologous to the linking sequence, and (ii) when the linking sequence does not have at least about 80% sequence identity to a Notch JMD or a Notch JMD wherein the LNR and/or an HD of a Notch receptor has been deleted, the transmembrane domain is not a Notch1 transmembrane domain.

### Extracellular Domain (ECD)

In some embodiments, the ECD of the chimeric polypeptides receptors disclosed herein has a binding affinity for one or more target ligands. In principle, there are no particular limitations with regard to suitable ligands that may be targeted by the chimeric polypeptides provided herein. In some embodiments, the target ligand is a cell-surface ligand. Non-limiting examples of suitable cell-surface ligands include cell surface receptors; adhesion proteins; carbohydrates, lipids, glycolipids, lipoproteins, and lipopolysaccharides that are surface-bound; integrins; mucins; and lectins. In some embodiments, the ligand is a protein. In some embodiments, the ligand is a carbohydrate. In some embodiments, the ligand is a cluster of differentiation (CD) marker. In some embodiments, the CD marker is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, BCMA (CD269), ALPPL2, GFP, eGFP, and SIRPα.

In some embodiments, the extracellular domain includes the ligand-binding portion of a receptor. In some embodiments, the extracellular domain includes an antigen-binding moiety that binds to one or more target antigens. In some embodiments, the antigen-binding moiety includes one or more antigen-binding determinants of an antibody or a functional antigen-binding fragment thereof. One skilled in the art upon reading the present disclosure will readily understand that the term “functional fragment thereof” or “functional variant thereof” refers to a molecule having biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. For example, an antibody capable of binding to an epitope of a cell surface receptor may be truncated at the N-terminus and/or C-terminus, and the retention of its epitope binding activity assessed using assays known to those of skill in the art. In some embodiments, the antigen-binding moiety is selected from the group consisting of an antibody, a nanobody, a diabody, a triabody, or a minibody, a F(ab′)₂ fragment, a Fab fragment, a single chain variable fragment (scFv), and a single domain antibody (sdAb), or a functional fragment thereof. In some embodiments, the antigen-binding moiety includes an scFv.

The antigen-binding moiety can include naturally-occurring amino acid sequences or can be engineered, designed, or modified so as to provide desired and/or improved properties, e.g., binding affinity. Generally, the binding affinity of an antigen-binding moiety, e.g., an antibody, for a target antigen (e.g., CD19 antigen) can be calculated by the Scatchard method described by Frankel et al., Mol Immunol (1979) 16:101-06. In some embodiments, binding affinity is measured by an antigen/antibody dissociation rate. In some embodiments, binding affinity is measured by a competition radioimmunoassay. In some embodiments, binding affinity is measured by ELISA. In some embodiments, antibody affinity is measured by flow cytometry. An antibody that “selectively binds” an antigen (such as CD19) is an antigen-binding moiety that binds the antigen with high affinity and does not significantly bind other unrelated antigens.

A skilled artisan can select an extracellular domain based on the desired localization or function of a cell that is genetically modified to express a chimeric polypeptide or synthetic Notch receptor of the present disclosure. For example, the extracellular domain can be selected to target a receptor-expressing cell to estrogen-dependent breast cancer cells. In some embodiments, the extracellular domain of the disclosed chimeric polypeptide Notch receptors is capable of binding a tumor-associated antigen (TAA) or a tumor-specific antigen (TSA). A skilled artisan in the art will understand that TAAs include a molecule, such as, for example, a protein, present on tumor cells and on a sub-population of normal cells, or on many normal cells, but at much lower concentration than on tumor cells. Examples include, without limitation, CEA, AFP, HER2, CTAG1B and MAGEA1. In contrast, TSAs generally include a molecule, such as, e.g., a protein, present on tumor cells but not expressed on normal cells. Examples include, without limitation, oncoviral antigens and mutated proteins (also known as neoantigens).

In some cases, the antigen-binding moiety is specific for an epitope present in an antigen that is expressed by a tumor cell, i.e., a tumor-associated antigen or a tumor-specific antigen. The tumor-associated or tumor-specific antigen can be an antigen associated with, e.g., a breast cancer cell, a B cell lymphoma, a pancreatic cancer, a Hodgkin lymphoma cell, an ovarian cancer cell, a prostate cancer cell, a mesothelioma, a lung cancer cell, a non-Hodgkin B-cell lymphoma (B-NHL) cell, an ovarian cancer cell, a prostate cancer cell, a mesothelioma cell, a melanoma cell, a chronic lymphocytic leukemia cell, an acute lymphocytic leukemia cell, a neuroblastoma cell, a glioma, a glioblastoma, a colorectal cancer cell, and the like. It will also be understood that a tumor-associated antigen may also be expressed by a non-cancerous cell. In some embodiments, the antigen-binding domain is specific for an epitope present in a tissue-specific antigen. In some embodiments, the antigen-binding domain is specific for an epitope present in a disease-associated antigen.

Generally, there are no particular limitations with regard to suitable surface antigens that may be targeted by the chimeric polypeptide receptors disclosed herein. Non-limiting examples of suitable target antigens include CD19, B7H3 (CD276), BCMA (CD269), alkaline phosphatase, placental-like 2 (ALPPL2), green fluorescent protein (GFP), enhanced green fluorescent protein (EGFP), signal regulatory protein α (SIRPα), CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MUC1, NCAM, PAP, PDGFR-β, PRSS21, PSCA, PSMA, ROR1, SSEA-4, TAG72, TEM1/CD248, TEM7R, TSHR, VEGFR2, ALPI, citrullinated vimentin, cMet, and Axl.

In some embodiments, the target antigen is selected from CD19, B7H3 (CD276), BCMA (CD269), CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MUC1, NCAM, PAP, PDGFR-β, PRSS21, PSCA, PSMA, ROR1, SSEA-4, TAG72, TEM1/CD248, TEM7R, TSHR, VEGFR2, ALPI, citrullinated vimentin, cMet, Axl, GPC2, human epidermal growth factor receptor 2 (Her2/neu), CD276 (B7-H3), IL-13Rα1, IL-13Rα2, alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD123, CD93, CD99, CD 117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11α), myostatin, OX-40, scleroscin, SOST, TGFβ1, TNF-α, VEGF-A, pyruvate kinase isoenzyme type M2 (tumor M2-PK), CD20, CD5, CD7, CD3, TRBC1, TRBC2, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin.

Further non-limiting examples of suitable antigens include PAP (prostatic acid phosphatase), prostate stem cell antigen (PSCA), prostein, NKG2D, TARP (T cell receptor γ alternate reading frame protein), Trp-p8, STEAP1 (six-transmembrane epithelial antigen of the prostate 1), an abnormal ras protein, an abnormal p53 protein, integrin β3 (CD61), galactin, K-Ras (V-Ki-ras2 Kirsten rat sarcoma viral oncogene), Ral-B, GPC2, CD276 (B7-H3), or IL-13Rα. In some embodiments, the antigen is Her2. In some embodiments, the antigen is ALPPL2. In some embodiments, the antigen is BCMA. In some embodiments, the antigen-binding moiety of the ECD is specific for a reporter protein, such as GFP and eGFP. Non-limiting examples of such antigen binding moiety include a LaG17 anti-GFP nanobody. In some embodiments, the antigen-binding moiety of the ECD includes an anti-BCMA fully-humanized VH domain (FHVH). In some embodiments, the antigen is signal regulatory protein α (SIRPα).

Additional antigens that can be suitable for the chimeric polypeptide receptors disclosed herein include, but are not limited to GPC2, human epidermal growth factor receptor 2 (Her2/neu), CD276 (B7-H3), IL-13Rα1, IL-13Rα2, α-fetoprotein (AFP), carcinoembryonic antigen (CEA), cancer antigen-125 (CA-125), CA19-9, calretinin, MUC-1, epithelial membrane protein (EMA), epithelial tumor antigen (ETA). Other suitable target antigens include, but are not limited to, tyrosinase, melanoma-associated antigen (MAGE), CD34, CD45, CD123, CD93, CD99, CD 117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1.

Additional suitable antigens include, but are not limited to, those associated with an inflammatory disease such as, AOC3 (VAP-1), CAM-3001, CCL11 (eotaxin-1), CD125, CD147 (basigin), CD154 (CD40L), CD2, CD20, CD23 (IgE receptor), CD25 (one subunit of the heterodimeric IL-2 receptor), CD3, CD4, CD5, IFNα, IFNγ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11α), myostatin, OX-40, scleroscin, SOST, TGFβ1, TNFα, and VEGF-A.

Further antigens suitable for the chimeric polypeptides and synthetic Notch receptors disclosed herein include, but are not limited to the pyruvate kinase isoenzyme type M2 (tumor M2-PK), CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin. Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate-1), an abnormal ras protein, an abnormal p53 protein, integrin β3 (CD61), galactin, K-Ras (V-Ki-ras2 Kirsten rat sarcoma viral oncogene), and Ral-B. In some embodiments, the antigen is ALPPL2. In some embodiments, the antigen is BCMA. In some embodiments, the antigen-binding moiety of the ECD is specific for a reporter protein, such as GFP and eGFP. Non-limiting examples of such antigen binding moiety include a LaG17 anti-GFP nanobody. In some embodiments, the antigen-binding moiety of the ECD includes an anti-BCMA fully-humanized VH domain (FHVH). In some embodiments, the antigen is signal regulatory protein α (SIRPα).

In some embodiments, antigens suitable for targeting by the chimeric polypeptides and chimeric receptors disclosed herein include ligands derived from a pathogen. For example, the antigen can be HER2 produced by HER2-positive breast cancer cells. In some embodiments, the antigen can be CD19 that is expressed on B-cell leukemia. In some embodiments, the antigen can be EGFR that is expressed on glioblastoma multiform (GBM) but much less expressed so on healthy CNS tissue. In some embodiments, the antigen can be CEA that is associated with cancer in adults, for example colon cancer.

In some embodiments, the antigen-binding moiety of the extracellular domain is specific for a cell surface target, where non-limiting examples of cell surface targets include CD19, CD30, Her2, CD22, ENPP3, EGFR, CD20, CD52, CD11α, and α-integrin. In some embodiments, the chimeric polypeptides and synthetic Notch receptors disclosed herein include an extracellular domain having an antigen-binding moiety that binds CD19, CEA, HER2, MUC1, CD20, ALPPL2, BCMA, or EGFR. In some embodiments, the chimeric polypeptides provided herein include an extracellular domain including an antigen-binding moiety that binds ALPPL2. In some embodiments, the chimeric polypeptides provided herein include an extracellular domain including an antigen-binding moiety that binds BCMA. In some embodiments, the chimeric polypeptides provided herein include an extracellular domain including an antigen-binding moiety that binds Her2. In some embodiments, the chimeric polypeptides and synthetic Notch receptors disclosed herein include an extracellular domain including an antigen-binding moiety that binds CD19, ALPPL2, BCMA, or Her2. In some embodiments, the antigen-binding moiety includes an amino acid sequence having at least 70%, at least 75%, at least 80%, at least 85%, at least 90%, at least 95%, at least 96%, at least 97%, at least 98%, at least 99% sequence identity to SEQ ID NO: 95 in the Sequence Listing. In some embodiments, the antigen-binding moiety includes an amino acid sequence having at least 90% sequence identity to SEQ ID NO: 95. In some embodiments, the antigen-binding moiety includes an amino acid sequence having at least 95% sequence identity to SEQ ID NO: 95. In some embodiments, the antigen-binding moiety includes an amino acid sequence having 100% sequence identity to SEQ ID NO: 95. In some embodiments, the antigen-binding moiety includes the amino acid sequence of SEQ ID NO: 5, wherein one, two, three, four, or five of the amino acid residues in SEQ ID NO: 95 is/are substituted by a different amino acid residue.

### Linking Sequence/JMD

As described above, the chimeric polypeptides and receptors of the disclosure include a linking polypeptide sequence disposed between the extracellular ligand-binding domain (ECD) and the TMD.

In some embodiments, the Notch JMD, including the NRR and HD, is used as the linking polypeptide. In some embodiments, the linking polypeptide of the chimeric receptors has at least about 80% amino acid sequence identity to a Notch juxtamembrane domain (JMD). In some embodiments, the linking polypeptide of the disclosed chimeric receptors has at least about 80% amino acid sequence identity to a Notch JMD wherein the LNR and/or an HD of a Notch receptor has been deleted. In some embodiments, the linking polypeptide of the chimeric receptors has at least about 80% amino acid sequence identity to a polypeptide hinge domain.

In some embodiments, one or more domains of a ROBO (Roundabout) cell surface receptor are incorporated into the linking polypeptide of the chimeric receptors of the present disclosure. ROBO receptors, in a manner similar to Notch, release a nuclear transcription factor domain following ligand-induced cleavage of the extracellular portion of the receptor by the ADAM10 MMP and γ-secretase. However, ROBO does not contain a LIN/Notch domain, EGF-like repeats, or an HD domain. Additionally, the primary ligand for ROBO is a soluble protein (SLIT). Mammals have four ROBO receptors: ROBO1-3 have five immunoglobulin-like (Ig) domains, three fibronectin (Fn) repeats, and a transmembrane domain linked to an intracellular domain. ROBO4 has only two Ig domains, and two Fn domains. Additional information in this regard can be found in, for example, H. Blockus et al., Development (2016) 143:3037-44, which is hereby incorporated by reference.

In some embodiments, the linking polypeptide of the chimeric receptors as disclosed herein includes at least one fibronectin (Fn) repeat derived from a ROBO receptor. The linking polypeptide of the disclosed chimeric receptors can contain 1, 2, 3, 4, or 5 Fn repeats. In some embodiments, the linking polypeptide of the chimeric receptors includes about 1 to about 5 Fn repeats, about 1 to about 3 Fn repeats, or about 2 to about 3 Fn repeats. In some embodiments, the linking polypeptide of the chimeric receptors has at least about 80% amino acid sequence identity to a ROBO1 JMD including at least one fibronectin repeat.

In some embodiments, the linking polypeptide of the chimeric receptors has a polypeptide sequence of about 4 to about 40 amino acid residues in length (e.g., 4, 5, 6, 7, 8, 9, 10, 11, 12, 13, 14, 15, 16, 17, 18, 19, 20, or more amino acid residues). In some embodiments, the length and amino acid composition of the linking polypeptide can be optimized to vary the orientation and/or proximity of the ECD and the TMD relative to one another to achieve a desired activity of the chimeric polypeptide of the disclosure. In some embodiments, the linking polypeptide of the chimeric receptors has a polypeptide sequence of about 4 to about 40, about 5 to about 30, about 10 to about 20, about 10 to about 30, about 10 to about 40, about 5 to about 20, about 5 to about 40, or about 20 to about 40 amino acid residues in length.

In some embodiments, the linking polypeptide comprises or consists of an amino acid sequence having at least about 80% sequence identity, such as, at least 80%, at least 85%, at least 90%, at least 95%, at least 96%, at least 97%, at least 98%, or about 99% sequence identity to a sequence set forth in SEQ ID NOS: 97-99 or 138-148 in the Sequence Listing. In some embodiments, the linking polypeptide comprises or consists of an amino acid sequence having at least about 80% sequence identity to a sequence set forth in SEQ ID NOS: 97-99 or 138-148. In some embodiments, the linking polypeptide comprises or consists of an amino acid sequence having at least about 90% sequence identity to a sequence set forth in SEQ ID NOS: 97-99 or 138-148. In some embodiments, the linking polypeptide comprises or consists of an amino acid sequence having at least about 95% sequence identity to a sequence set forth in SEQ ID NOS: 97-99 or 138-148. In some embodiments, the linking polypeptide comprises or consists of an amino acid sequence having about 100% sequence identity to a sequence set forth in SEQ ID NOS: 97-99 or 138-148. In some embodiments, the linking polypeptide comprises or consists of an amino acid sequence selected from the group consisting of SEQ ID NOS: 97-99 or 138-148, wherein one, two, three, four, or five of the amino acid residues in any one of SEQ ID NO: 97-99 and 138-148 is/are substituted by a different amino acid residues.

### Transmembrane Domain (TMD)

As described above, the chimeric receptors of the disclosure include a transmembrane domain having at least about 80% sequence identity to the TMD of a Type 1 transmembrane receptor and comprising one or more ligand-inducible proteolytic cleavage sites.

Examples of proteolytic cleavage sites in a Notch receptor (e.g., S2 or S3) are as described above. Additional proteolytic cleavage sites suitable for the compositions and methods disclosed herein include, but are not limited to, a metalloproteinase cleavage site for a matrix metalloproteinase (MMP) selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue such as Leu, Ile, Val, Phe, Trp, Tyr, Val, Met, and Pro) (SEQ ID NO: 103), e.g., Pro-X-X-Hy-(Ser/Thr) (SEQ ID NO: 104), e.g., Pro-Leu/Gln-Gly-Met-Thr-Ser (SEQ ID NO: 105) or Pro-Leu/Gln-Gly-Met-Thr (SEQ ID NO: 106). Another example of a suitable protease cleavage site is a plasminogen activator cleavage site, e.g., a urokinase plasminogen activator (uPA) or a tissue plasminogen activator (tPA) cleavage site. Another example of a suitable protease cleavage site is a prolactin cleavage site. Specific examples of cleavage sequences of uPA and tPA include sequences comprising Val-Gly-Arg (SEQ ID NO: 107). Another example of a protease cleavage site that can be included in a proteolytically cleavable linker is a tobacco etch virus (TEV) protease cleavage site, e.g., Glu-Asn-Leu-Tyr-Thr-Gln-Ser (SEQ ID NO: 108), where the protease cleaves between the glutamine and the serine. Another example of a protease cleavage site that can be included in a proteolytically cleavable linker is an enterokinase cleavage site, e.g., Asp-Asp-Asp-Asp-Lys (SEQ ID NO: 109), where cleavage occurs after the lysine residue. Another example of a protease cleavage site that can be included in a proteolytically cleavable linker is a thrombin cleavage site, e.g., Leu-Val-Pro-Arg (SEQ ID NO: 110). Additional suitable linkers comprising protease cleavage sites include sequences cleavable by the following proteases: a PreScission™ protease (a fusion protein comprising human rhinovirus 3C protease and glutathione-S-transferase), a thrombin, cathepsin B, Epstein-Barr virus protease, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrix metalloproteinase (MT-MMP), stromelysin 3 (or MMP-11), thermolysin, fibroblast collagenase and stromelysin-1, matrix metalloproteinase 13 (collagenase-3), tissue-type plasminogen activator (tPA), human prostate-specific antigen, kallikrein (hK3), neutrophil elastase, and calpain (calcium activated neutral protease). Proteases that are not native to the host cell in which the receptor is expressed (for example, TEV) can be used as a further regulatory mechanism, in which activation of the synthetic Notch receptor of the disclosure is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

Generally, the TMD suitable for the chimeric receptors disclosed herein can be any transmembrane domain of a Type 1 transmembrane receptor comprising at least one γ-secretase cleavage site. Detailed description of the structure and function of the γ-secretase complex as well as its substrate proteins, including amyloid precursor protein (APP) and Notch, can, for example, be found in a recent review by Zhang et al., Frontiers Cell Neurosci (2014). Non-limiting suitable TMDs from Type 1 transmembrane receptors include those from CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, and IFNAR2, wherein the TMD includes at least one γ-secretase cleavage site. Additional TMDs suitable for the compositions and methods described herein include, but are not limited to, transmembrane domains from Type 1 transmembrane receptors IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKHD1, NECTIN1, PCDHGC3, NRG1, LRP1B, CDH2, NRG2, PTPRK, SCN2B, Nradd, and PTPRM. In some embodiments, the TMD of the chimeric polypeptides or Notch receptors of the disclosure is a TMD derived from the TMD of a member of the calsyntenin family, such as, alcadein alpha and alcadein gamma. In some embodiments, the TMD of the chimeric polypeptides or Notch receptors of the disclosure is a TMD derived from a different Notch receptor. For example, in a synthetic Notch receptor based on human Notch1, the Notch1 TMD can be substituted with a human Notch2 TMD, human Notch3 TMD, human Notch4 TMD, or a Notch TMD from a non-human animal such as Danio rerio, Drosophila melanogaster, Xenopus laebis, or Gallus.

Accordingly, in some embodiments, the transmembrane domain includes an amino acid sequence exhibiting at least 70%, at least 75%, at least 80%, at least 85%, at least 90%, at least 95%, at least 96%, at least 97%, at least 98%, at least 99% sequence identity to a polypeptide sequence having at least about 70% sequence identity to a transmembrane domain from a Type 1 transmembrane receptor that comprises a γ-secretase cleavage site. In some embodiments, the transmembrane domain includes an amino acid sequence exhibiting at least 70% sequence identity to a transmembrane domain from a Type 1 transmembrane receptor that comprises a γ-secretase cleavage site. In some embodiments, the transmembrane domain includes an amino acid sequence exhibiting at least about 80% sequence identity to a transmembrane domain from a Type 1 transmembrane receptor that comprises a γ-secretase cleavage site. In some embodiments, the transmembrane domain includes an amino acid sequence exhibiting at least about 90% sequence identity to a transmembrane domain from a Type 1 transmembrane receptor that comprises a γ-secretase cleavage site. In some embodiments, the transmembrane domain includes an amino acid sequence exhibiting at least about 95% sequence identity to a transmembrane domain from a Type 1 transmembrane receptor that comprises a γ-secretase cleavage site. In some embodiments, the Type 1 transmembrane receptor is selected from the group consisting of CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, IFNAR2, IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKHD1, NECTIN1, PCDHGC3, NRG1, LRP1B, CDH2, NRG2, PTPRK, SCN2B, Nradd, and PTPRM. In some embodiments, the TMD includes an amino acid sequence exhibiting at least about 70%, at least about 75%, at least about 80%, at least about 85%, at least about 90%, at least about 95%, at least about 96%, at least about 97%, at least about 98%, or at least about 99% sequence identity to any one of SEQ ID NOS: 1-94 in the Sequence Listing. In some embodiments, the transmembrane domain includes an amino acid sequence having at least about 90% sequence identity to SEQ ID NOS: 1-94. In some embodiments, the transmembrane domain includes an amino acid sequence having at least about 95% sequence identity to SEQ ID NOS: 1-94. In some embodiments, the transmembrane domain includes an amino acid sequence having about 100% sequence identity to SEQ ID NOS: 1-94. In some embodiments, the transmembrane domain includes the amino acid sequence of SEQ ID NO: 1-94, wherein one, two, three, four, or five of the amino acid residues in SEQ ID NO: 10 is/are substituted by a different amino acid residue.

In some embodiments, the amino acid substitution(s) within the TMD includes one or more substitutions within a “GV” motif of the TMD. In some embodiments, at least one of such substitution(s) comprises a substitution to alanine. For example, one, two, three, four, five, or more of the amino acid residues of the sequence FMYVAAAAFVLLFFVGCGVLLS (SEQ ID NO: 57), as well as any one of sequences as set forth in SEQ ID NOS: 1 and 2, may be substituted by a different amino acid residue. In some embodiments, the amino acid residue at position 18 and/or 19 of the “GV” motif within SEQ ID NO: 57 is substituted by a different amino acid residue. In some embodiments, the glycine residue at position 18 of SEQ ID NO: 57 is substituted by a different amino acid residue. In some embodiments, the valine residue at position 19 of SEQ ID NO: 57 is substituted by a different amino acid residue. In some embodiments, the transmembrane domain comprises an amino acid sequence having a sequence corresponding to SEQ ID NO: 57 with a mutation at the position corresponding to position 18 of SEQ ID NO: 57, such as G18A mutations. In some embodiments, the transmembrane domain comprises an amino acid sequence having a sequence corresponding to SEQ ID NO: 57 with a mutation at the position corresponding to position 19 of SEQ ID NO: 57, such as V19A mutations.

### Stop-Transfer Sequence (STS)

In some embodiments, the chimeric receptors of the disclosure include a stop-transfer sequence (STS), which is essentially a highly-charged domain located between the TMD and the ICD. Without being bound to any particular theory, such a highly-charged domain disposed between the TMD and the ICD prevents the ICD from entering the membrane. In principle, there are no particular limitations to the length and/or amino acid composition of the STS. In some embodiments, any arbitrary single-chain peptide comprising about 1 to about 40 amino acid residues (e.g., 1, 2, 3, 4, 5, 6, 7, 8, 9, 10, 11, 12, 13, 14, 15, 16, 17, 18, 19, 20, or more amino acid residues) can be used as an STS. In some embodiments, the STS includes about 1 to 15, about 5 to 20, about 8 to 25, about 10 to 30, about 12 to 35, about 14 to 40, about 5 to 40, about 10 to 35, about 15 to 30, about 20 to 25, about 20 to 40, about 10 to 30, about 4 to 20, or about 5 to 25 amino acid residues. In some embodiments, the STS includes about 1 to 10, about 5 to 12, about 6 to 14, about 7 to 18, about 8 to 20, about 9 to 22, about 10 to 24, or about 11 to 26 amino acid residues. In some embodiments, the STS includes about 4 to 10 residues, such as, 4, 5, 6, 7, 8, 9, or 10 amino acid residues. In some embodiments, the STS includes a sequence having at least 80% sequence identity, such as, at least 80%, at least 85%, at least 90%, at least 95%, at least 96%, at least 97%, at least 98%, or 99% sequence identity to SEQ ID NO: 96, 135, 136, or 137 in the Sequence Listing. In some embodiments, the STS includes an amino acid sequence having at least 90% sequence identity to SEQ ID NO: 96, 135, 136, or 137. In some embodiments, the STS includes an amino acid sequence having at least 95% sequence identity to SEQ ID NO: 96, 135, 136, or 137. In some embodiments, the STS includes an amino acid sequence having at least 100% sequence identity to SEQ ID NO: 96, 135, 136, or 137. In some embodiments, the STS includes the amino acid sequence of SEQ ID NO: 96, 135, 136, or 137, wherein one, two, three, four, or five of the amino acid residues in SEQ ID NO: 96, 135, 136, or 137 is/are substituted by a different amino acid residue.

In some embodiments, the STS includes a sequence having at least 70% sequence identity, such as, at least 70%, at least 75%, at least 80%, at least 85%, at least 90%, at least 95%, at least 96%, at least 97%, at least 98%, or 99% sequence identity to a STS sequence from Notch1, Notch2, Notch3, Notch4, CSF1R, CXCL16, DAG1, GHR, PTPRF, AGER, KL, NRG1, LRP1B, Jag2, EPCAM, KCNE3, CDH2, NRG2, PTPRK, BTC, EPHA3, IL1R2, or PTPRM. In some embodiments, the STS includes a sequence comprising only Lys (K) or Arg (R) in the first 4 residues. In some embodiments, the STS includes one, two, three, four, five, or more basic residues. In some embodiments, the STS includes five, four, three, two, one, or zero aromatic residues or residues with hydrophobic and/or bulky side chains.

### Intracellular Domain (ICD)

The chimeric receptor of the disclosure further comprises an intracellular domain (ICD) comprising a transcriptional regulator. The transcriptional regulator is a biochemical element that acts to activate or repress the transcription of a promoter-driven DNA sequence. Transcriptional regulators suitable for the compositions and methods of the disclosure can be naturally-occurring transcriptional regulators or can be engineered, designed, or modified so as to provide desired and/or improved properties, e.g., modulating transcription. In some embodiments, the transcriptional regulator directly regulates expression of one or more genes involved in differentiation of the cell. In some embodiments, the transcriptional regulator indirectly modulates expression of one or more genes involved in differentiation of the cell by modulating the expression of a second transcription factor which in turn modulates expression of one or more genes involved in differentiation of the cell. It will be understood by a skilled artisan that a transcriptional regulator can be a transcriptional activator or a transcriptional repressor. In some embodiments, the transcriptional regulator is a transcriptional repressor. In some embodiments, the transcriptional regulator is a transcriptional activator. In some embodiments, the transcriptional regulator can further include a nuclear localization signal. In some embodiments, the transcriptional regulator is selected from Gal4-VP16, Gal4-VP64, tetR-VP64, ZFHD1-VP64, Gal4-KRAB, and HAP1-VP16. In some embodiments, the transcriptional regulator is Gal4-VP64.

### Additional Domains

In some embodiments, the extracellular domains located N-terminally to the TMD can further include an additional domain, for example a membrane localization signal such as a CD8α signal, a detectable marker such as a myc tag or his tag, and the like. In some embodiments, the chimeric receptors further comprise one or more additional proteolytic cleavage sites. In some embodiments, the chimeric receptors do not comprise an additional proteolytic cleavage site. In some embodiments, the chimeric receptors further comprise one or more glycosylation sites. In some embodiments, the chimeric receptors do not comprise a glycosylation site. In some embodiments, the chimeric receptors do not comprise a hinge domain for promoting oligomerization of the chimeric polypeptide via intermolecular disulfide bonding.

In some embodiments, the chimeric receptors further comprise elements of the highly conserved ROBO cell surface receptors. In some embodiments, the chimeric receptors further comprise an extracellular oligomerization domain (e.g., a hinge domain) to promote oligomerization, e.g., dimerization, trimerization, or higher order multimers of the chimeric receptor. In some embodiments, the chimeric receptors disclosed comprise, from N-terminus to C-terminus: (a) an extracellular ligand binding domain having a binding affinity for a selected ligand; (b) a ROBO domain comprising a fibronectin repeat; (c) a TMD including one or more ligand-inducible proteolytic cleavage sites; and (d) an ICD including a transcriptional regulator, wherein binding of the selected ligand to the extracellular binding domain induces cleavage at the ligand-inducible proteolytic cleavage site in the TMD, and wherein the chimeric polypeptide does not include a Notch negative regulatory region (NRR), LNR, or an HD of a Notch receptor.

In some embodiments, the hinge domain includes polypeptide motifs capable of promoting oligomer formation of the chimeric polypeptide via intermolecular disulfide bonding. In some embodiments, a chimeric receptor disclosed herein includes, from N-terminus to C-terminus: (a) an extracellular ligand binding domain having a binding affinity for a selected ligand; (b) a hinge domain capable of promoting oligomer formation of the chimeric polypeptide via intermolecular disulfide bonding; (c) a TMD comprising one or more ligand-inducible proteolytic cleavage sites; and (d) an ICD comprising a transcriptional regulator, wherein binding of the selected ligand to the extracellular binding domain induces cleavage at a ligand-inducible proteolytic cleavage sites in the TMD, and wherein the chimeric polypeptide does not comprise an extracellular subunit (NEC) of a Notch receptor or an NRR or HD of a Notch receptor.

Chimeric receptors of the present disclosure can be chimeric polypeptides of any length, including chimeric polypeptides that are generally between about 100 amino acids (aa) to about 1000 aa, e.g., from about 100 aa to about 200 aa, from about 150 aa to about 250 aa, from about 200 aa to about 300 aa, from about 250 aa to about 350 aa, from about 300 aa to about 400 aa, from about 350 aa to about 450 aa, from about 400 aa to about 500 aa in length. In some embodiments, the disclosed chimeric polypeptides are generally between about 400 aa to about 450 aa, from about 450 aa to about 500 aa, from about 500 aa to about 550 aa, from about 550 aa to about 600 aa, from about 600 aa to about 650 aa, from about 650 aa to about 700 aa, from about 700 aa to about 750 aa, from about 750 aa to about 800 aa, from about 800 aa to about 850 aa, from about 850 aa to about 900 aa, from about 900 aa to about 950 aa, or from about 950 aa to about 1000 aa in length. In some cases, the chimeric polypeptides of the present disclosure have a length of from about 300 aa to about 400 aa. In some cases, the chimeric polypeptides of the present disclosure have a length of from 300 aa to 350 aa. In some cases, the chimeric polypeptides of the present disclosure have a length of from 300 aa to 325 aa. In some cases, the chimeric polypeptides of the present disclosure have a length of from 350 aa to 400 aa. In some cases, the chimeric polypeptides of the present disclosure have a length of from 750 aa to 850 aa.

In some embodiments, the chimeric receptors of the disclosure include: (a) an extracellular ligand-binding domain (b) a linking polypeptide including an amino acid sequence having at least about 80%, 90%, 95%, 96%, 97, 98%, 99%, or 100% sequence identity to any one of SEQ ID NO: 97-99 and 138-148; (c) a TMD including an amino acid sequence having at least about 80%, 90%, 95%, 96%, 97, 98%, 99%, or 100% sequence identity to any one of SEQ ID NOS: 1-94; (d) an STS including an amino acid sequence having at about least 80%, 90%, 95%, 96%, 97, 98%, 99%, or 100% sequence identity to SEQ ID NO: 95; and (e) an ICD comprising a transcriptional regulator. In some embodiments, the chimeric receptor of the disclosure include: (a) a linking polypeptide including an amino acid sequence having at least about 90% sequence identity to any one of SEQ ID NO: 97-99 and 138-148; (b) a TMD including an amino acid sequence having at least about 90% sequence identity to any one of SEQ ID NOS: 1-94; and (c) an STS including an amino acid sequence having at least about 90% sequence identity to SEQ ID NO: 95. In some embodiments, the chimeric receptors of the disclosure include: (a) a linking sequence including an amino acid sequence having at least about 95% sequence identity to any one of SEQ ID NO: 97-99 and 138-148; (b) a transmembrane domain including an amino acid sequence having at least about 95% sequence identity to any one of SEQ ID NO: 1-94; and (c) an STS including an amino acid sequence having at least about 95% sequence identity to SEQ ID NO: 95.

In some embodiments, the chimeric polypeptides of the disclosure include an amino acid sequence having at least about 80%, 90%, 95%, 96%, 97, 98%, 99%, or 100% sequence identity to a chimeric receptor disclosed herein. In some embodiments, the chimeric polypeptides of the disclosure include an amino acid sequence having at least about 80%, 90%, 95%, 96%, 97, 98%, 99%, or 100% sequence identity to a sequence selected from the group consisting of SEQ ID NO: 111-134.

### Nucleic Acid Molecules

In one aspect, some embodiments disclosed herein relate to nucleic acid molecules comprising nucleotide sequences encoding the chimeric polypeptides and Notch receptors of the disclosure, including expression cassettes, and expression vectors containing these nucleic acid molecules operably linked to heterologous nucleic acid sequences such as, for example, regulatory sequences which direct in vivo expression of the receptor in a host cell.

Nucleic acid molecules of the present disclosure can be nucleic acid molecules of any length, including nucleic acid molecules that are generally between about 5 Kb and about 50 Kb, for example between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, the nucleic acid molecules comprise a nucleotide sequence encoding a chimeric receptor comprising, from N-terminus to C-terminus: (a) an extracellular ligand binding domain having a binding affinity for a selected ligand; (b) a linking polypeptide having: (i) at least about 80% sequence identity to a Notch JMD; (ii) at least about 80% sequence identity to a Notch JMD wherein the LNR and/or an HD of a Notch receptor has been deleted; (iii) at least about 80% sequence identity to a polypeptide hinge domain; (iv) at least about 80% sequence identity to a ROBO1 JMD including at least one fibronectin (Fn) repeat; or (v) a polypeptide having about 2 to about 40 amino acids; (c) a TMD having at least about 80% sequence identity to the transmembrane domain of a Type 1 transmembrane receptor comprising one or more ligand-inducible proteolytic cleavage sites; and (d) an ICD comprising a transcriptional regulator, wherein binding of the selected ligand to the extracellular binding domain induces cleavage at the ligand-inducible proteolytic cleavage site between the transcriptional regulator and the linking sequence, and wherein (i) when the linking sequence has at least about 80% sequence identity to a Notch JMD or a SynNotch JMD wherein the NRR and HD has been deleted, the TMD is heterologous to the linking sequence, and (ii) when the linking sequence does not have at least about 80% sequence identity to a Notch JMD or a Notch JMD wherein the NRR and HD has been deleted, the TMD is not a Notch1 TMD.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell or tissue and/or into an individual. Thus, in some embodiments, an expression cassette of the disclosure comprises a nucleotide sequence encoding a chimeric polypeptide operably linked to expression control elements sufficient to guide expression of the cassette in vivo. In some embodiments, the expression control element comprises a promoter and/or an enhancer and optionally, any or a combination of other nucleic acid sequences capable of effecting transcription and/or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus.

In some embodiments, the recombinant nucleic acids encode a polypeptide with an amino acid sequence having at least about 80%, 90%, 95%, 96%, 97, 98%, 99%, or 100% sequence identity to a chimeric receptor disclosed herein. In some embodiments, recombinant nucleic acids encode a polypeptide with an amino acid sequence having at least about 80%, 90%, 95%, 96%, 97, 98%, 99%, or 100% sequence identity to a chimeric polypeptide which includes an amino acid sequence having at least about 80%, 90%, 95%, 96%, 97, 98%, 99%, or 100% sequence identity to a sequence selected from the group consisting of SEQ ID NO: 111-134.

In some embodiments, the nucleic acid molecules encode a chimeric polypeptide comprising: (a) a linking polypeptide including an amino acid sequence having at least about 80%, 90%, 95%, 96%, 97, 98%, 99%, or 100% sequence identity to any one of SEQ ID NO: 97-99 and 138-148; (b) a transmembrane domain including an amino acid sequence having at least about 80%, 90%, 95%, 96%, 97, 98%, 99%, or 100% sequence identity to any one of SEQ ID NOS: 1-94; and (c) an STS including an amino acid sequence having at least about 80%, 90%, 95%, 96%, 97, 98%, 99%, or 100% sequence identity to SEQ ID NO: 95. In some embodiments, the nucleic acid molecules encode a chimeric polypeptide comprising: (a) a linking polypeptide including an amino acid sequence having at least about 90% sequence identity to any one of SEQ ID NO: 97-99 and 138-148; (b) a transmembrane domain including an amino acid sequence having at least about 90% sequence identity to any one of SEQ ID NOS: 1-94; and (c) an STS including an amino acid sequence having at least about 90% sequence identity to SEQ ID NO: 95. In some embodiments, the nucleic acid molecules encode a chimeric polypeptide comprising: (a) a linking sequence including an amino acid sequence having at least about 95% sequence identity to any one of SEQ ID NO: 97-99 and 138-148; (b) a transmembrane domain including an amino acid sequence having at least about 95% sequence identity to any one of SEQ ID NO: 1-94; and (c) an STS including an amino acid sequence having at least about 95% sequence identity to SEQ ID NO: 95.

The nucleic acid sequences encoding the chimeric receptors can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the chimeric receptors disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into an individual. An expression cassette can be inserted into a plasmid, cosmid, virus, autonomously replicating polynucleotide molecule, or bacteriophage, as a linear or circular, single-stranded or double-stranded, DNA or RNA polynucleotide, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any chimeric receptor disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, N.Y.: Cold Spring Harbor Laboratory and Sambrook, J., & Russel, D. W. (2001). Molecular Cloning: A Laboratory Manual (3rd ed.). Cold Spring Harbor, N.Y.: Cold Spring Harbor Laboratory (jointly referred to herein as “Sambrook”); Ausubel, F. M. (1987). Current Protocols in Molecular Biology. New York, N.Y.: Wiley (including supplements through 2014); Bollag, D. M. et al. (1996). Protein Methods. New York, N.Y.: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, Calif.: Academic Press; Lefkovits, I. (1997). The Immunology Methods Manual: The Comprehensive Sourcebook of Techniques. San Diego, Calif.: Academic Press; Doyle, A. et al. (1998). Cell and Tissue Culture: Laboratory Procedures in Biotechnology. New York, N.Y.: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, N.Y.: Cold Spring Harbor Laboratory Press; Beaucage, S. L. et al. (2000). Current Protocols in Nucleic Acid Chemistry. New York, N.Y.: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). For example, a chimeric receptor as disclosed herein can be produced in a eukaryotic host, such as a mammalian cells (e.g., COS cells, NIH 3T3 cells, or HeLa cells). These cells are available from many sources, including the American Type Culture Collection (Manassas, Va.). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally but encode the same gene product because the genetic code is degenerate. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., comprising either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by the polymerase chain reaction (PCR). In the event the nucleic acid molecule is a ribonucleic acid (RNA), transcripts can be produced, for example, by in vitro transcription.

### Recombinant Cells and Cell Cultures

The nucleic acid of the present disclosure can be introduced into a host cell, such as a human T lymphocyte, to produce a recombinant cell containing the nucleic acid molecule. Accordingly, some embodiments of the disclosure relate to methods for making recombinant cells, including the steps of: (a) providing a cell capable of protein expression and (b) contacting the provided cell with any of the recombinant nucleic acids described herein.

Introduction of the nucleic acid molecules of the disclosure into cells can be achieved by viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules are delivered to cells by viral or non-viral delivery vehicles known in the art. For example, the nucleic acid molecule can be stably integrated in the host genome, or can be episomally replicating, or present in the recombinant host cell as a mini-circle expression vector for a stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can be completed using classical random genomic recombination techniques or with more precise genome editing techniques such as using guide RNA directed CRISPR/Cas (such as CRISPR/Cas9), or DNA-guided endonuclease genome editing NgAgo (Natronobacterium gregoryi Argonaute), or TALENs genome editing (transcription activator-like effector nucleases). In some embodiments, the nucleic acid molecule present in the recombinant host cell as a mini-circle expression vector for a stable or transient expression.

The nucleic acid molecules can be encapsulated in a viral capsid or a lipid nanoparticle. For example, introduction of nucleic acids into cells may be achieved by viral transduction. In a non-limiting example, adeno-associated virus (AAV) is a non-enveloped virus that can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral systems are also suitable for nucleic acid delivery and gene therapy via viral transduction. Lentiviral vectors offer several attractive properties as gene-delivery vehicles, including: (i) sustained gene delivery through stable vector integration into host genome; (ii) the ability to infect both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) potentially safer integration site profile; and (vii) a relatively easy system for vector manipulation and production.

In some embodiments, host cells can be genetically engineered (e.g., transduced, transformed, or transfected) with, for example, a vector comprising a nucleic acid sequence encoding a chimeric receptor as described herein, either a virus-derived expression vector or a vector for homologous recombination further comprising nucleic acid sequences homologous to a portion of the genome of the host cell. Host cells can be either untransformed cells or cells that have already been transfected with one or more nucleic acid molecules.

In some embodiments, the recombinant cell is a prokaryotic cell or a eukaryotic cell. In some embodiments, the cell is transformed in vivo. In some embodiments, the cell is transformed ex vivo. In some embodiments, the cell is transformed in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell, a cytotoxic T cell, or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis performed on a sample obtained from a human subject.

In some embodiments, the recombinant cell further includes a second nucleic acid molecule as disclosed herein, wherein the first nucleic acid molecule and the second nucleic acid molecule do not have the same sequence. In some embodiments, the recombinant cell further includes a second chimeric polypeptide as disclosed herein, wherein the first chimeric polypeptide and the second chimeric polypeptide do not have the same sequence. In some embodiments, the first chimeric polypeptide modulates the expression and/or activity of the second chimeric polypeptide.

In some embodiments, the recombinant cell further includes an expression cassette encoding a protein of interest operably linked to a promoter, wherein expression of the protein of interest is modulated by the transcriptional regulator encoded by the chimeric receptor. In some embodiments, the protein of interest is heterologous to the recombinant cell. In principle, there are no particular limitations with regard to selecting proteins to target for modulation of expression by the transcriptional regulator encoded by the chimeric receptor. Non-limiting examples of proteins suitable for the regulation by the compositions and methods disclosed herein include cytokines, cytotoxins, chemokines, immunomodulators, pro-apoptotic factors, anti-apoptotic factors, hormones, differentiation factors, dedifferentiation factors, immune cell receptors, or reporter genes. In some embodiments, the immune cell receptor comprises a T-cell receptor (TCR). In some embodiments, the immune cell receptor comprises a chimeric antigen receptor (CAR). In some embodiments, the expression cassette encoding the protein of interest is incorporated into the same nucleic acid molecule that encodes the chimeric receptor of the disclosure. In some embodiments, the expression cassette encoding the protein of interest is incorporated into a second expression vector that is separate from the nucleic acid molecule encoding the chimeric receptor of the disclosure.

In another aspect, provided herein are various cell cultures including at least one recombinant cell as disclosed herein, and a culture medium. Generally, the culture medium can be any one of suitable culture media for the cell cultures described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the nucleic acids, and recombinant cells of the disclosure can be incorporated into compositions, including pharmaceutical compositions. Such compositions generally include the nucleic acids, and/or recombinant cells, and a pharmaceutically acceptable excipient, e.g., a carrier.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that it can be administered by syringe. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In some embodiments, the chimeric polypeptides and Notch receptors of the disclosure can also be administered by transfection or infection using methods known in the art, including but not limited to the methods described in McCaffrey et al. (Nature (2002) 418:6893), Xia et al. (Nature Biotechnol (2002) 20:1006-10), or Putnam (Am J Health Syst Pharm (1996) 53:151-60, erratum at Am J Health Syst Pharm (1996) 53:325).

### Methods of the Disclosure

Administration of any one or more of the therapeutic compositions described herein, e.g., nucleic acids, recombinant cells, and pharmaceutical compositions, can be used to treat individuals in the treatment of relevant health conditions or diseases, such as cancers and chronic infections. In some embodiments, the nucleic acids, recombinant cells, and pharmaceutical compositions are incorporated into therapeutic compositions for use in methods of treating an individual who has, who is suspected of having, or who may be at high risk for developing one or more autoimmune disorders or diseases associated with checkpoint inhibition. Exemplary autoimmune disorders and diseases can include, without limitation, celiac disease, type 1 diabetes, Graves' disease, inflammatory bowel disease, multiple sclerosis, psoriasis, rheumatoid arthritis, and systemic lupus erythematosus.

Accordingly, in one aspect, provided herein are methods for inhibiting an activity of a target cell in an individual, the methods comprising the step of administering to the individual a first therapy including one or more of the nucleic acids, recombinant cells, and pharmaceutical compositions provided herein, wherein the first therapy inhibits an activity of the target cell. For example, an activity of the target cell may be inhibited if its proliferation is reduced, if its pathologic or pathogenic behavior is reduced, if it is destroyed or killed, or the like. Inhibition includes a reduction of the measured quantity of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cell as disclosed herein, wherein the recombinant cell inhibits the target cell in the individual. Generally, the target cell of the disclosed methods can be any cell such as, for example an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cel]l, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell. In some embodiments, the target cell is a pathogenic cell.

In another aspect, provided herein are methods of treating a health condition (e.g., disease) in an individual in need thereof, the methods comprising a step of administering to the individual a first therapy including one or more of chimeric polypeptides, Notch receptors, nucleic acids, recombinant cells, or pharmaceutical compositions provided herein, wherein the first therapy treats the health condition in the individual. In some embodiments, the methods include administering to the individual a first therapy including an effective number of the recombinant cells provided herein, wherein the recombinant cells treat the health condition.

In another aspect, provided herein are methods for assisting in the treatment of a health condition (e.g., disease) in an individual in need thereof, the methods comprising the steps of administering to the individual a first therapy comprising one or more recombinant nucleic acids, recombinant cells, or pharmaceutical compositions as disclosed herein, and administering to the individual a second therapy, wherein the first and second therapies together treat the health condition in the individual. In some embodiments, the methods include administering to the individual a first therapy including an effective number of the recombinant cells as disclosed herein, wherein the recombinant cells treat the health condition.

### Administration of Recombinant Cells to an Individual

In some embodiments, the methods involve administering an effective amount or number of the recombinant cells of the disclosure to an individual who is in need of such method. This administering step can be accomplished using any method of implantation known in the art. For example, the recombinant cells of the disclosure can be injected directly into the individual's bloodstream by intravenous infusion or otherwise administered to the individual.

The terms “administering”, “introducing”, and “transplanting” are used interchangeably herein to refer to methods of delivering recombinant cells expressing the chimeric receptors provided herein to an individual. In some embodiments, the methods comprise administering recombinant cells to an individual by a method or route of administration that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even long-term engraftment for the life time of the individual.

When provided prophylactically, in some embodiments, the recombinant cells described herein are administered to an individual in advance of any symptom of a disease or condition to be treated. Accordingly, in some embodiments the prophylactic administration of a recombinant stem cell population serves to prevent the occurrence of symptoms of the disease or condition.

When provided therapeutically in some embodiments, recombinant stem cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source (i.e., the human subject being treated). In some embodiments, the recombinant cells are expanded in culture prior to administration to an individual in need thereof.

In some embodiments, the delivery of a composition comprising recombinant cells (i.e., a composition comprising a plurality of recombinant cells expressing any of the chimeric receptors provided herein) into an individual by a method or route results in at least partial localization of the cell composition at a desired site. A cell composition can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, instillation, and the like. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, administration by injection or infusion can be made.

In some embodiments, the recombinant cells are administered systemically, in other words a population of recombinant cells are administered other than directly into a target site, tissue, or organ, such that it enters, instead, the individual's circulatory system and, thus, is subject to metabolism and other like processes.

The efficacy of a treatment with a composition for the treatment of a disease or condition can be determined by the skilled clinician. However, one skilled in the art will appreciate that a treatment is considered effective treatment if any one or all of the signs or symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting disease progression, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment including a disclosed therapeutic composition for the treatment of disease can be determined by the skilled clinician. However, a treatment is considered effective if at least any one or all of the signs or symptoms of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; (2) relieving the disease, e.g., causing regression of symptoms; or (3) preventing or reducing the likelihood of the development of symptoms.

In some embodiments, the individual is a mammal. In some embodiments, the mammal is human. In some embodiments, the individual has or is suspected of having a disease associated with inhibition of cell signaling mediated by a cell surface ligand or antigen. The diseases suitable for being treated by the compositions and methods of the disclosure include, but are not limited to, cancers, autoimmune diseases, inflammatory diseases, and infectious diseases. In some embodiments, the disease is a cancer or a chronic infection.

### Additional Therapies

As discussed above, the recombinant cells, and pharmaceutical compositions described herein can be administered in combination with one or more additional therapeutic agents such as, for example, chemotherapeutics or anti-cancer agents or anti-cancer therapies. Administration “in combination with” one or more additional therapeutic agents includes simultaneous (concurrent) and consecutive administration in any order. In some embodiments, the one or more additional therapeutic agents, chemotherapeutics, anti-cancer agents, or anti-cancer therapies is selected from the group consisting of chemotherapy, radiotherapy, immunotherapy, hormonal therapy, toxin therapy, and surgery. “Chemotherapy” and “anti-cancer agent” are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

### Methods for Modulating an Activity of a Cell

In another aspect, provided herein are various methods for modulating an activity of a cell. The methods include: (a) providing a recombinant cell of the disclosure, and (b) contacting it with a selected ligand, wherein binding of the selected ligand to the extracellular binding domain induces cleavage of a ligand-inducible proteolytic cleavage site and releases the transcriptional regulator, wherein the released transcriptional regulator modulates an activity of the recombinant cell. One skilled in the art upon reading the present disclosure will appreciate that the disclosed methods can be carried out in vivo, ex vivo, or in vitro.

Activities of a cell that can be modulated using a method of the present disclosure include, but are not limited to, expression of a selected gene of the cell, proliferation of the cell, apoptosis of the cell, non-apoptotic death of the cell, differentiation of the cell, dedifferentiation of the cell, migration of the cell, secretion of a molecule from the cell, cellular adhesion of the cell, and cytolytic activity of the cell.

In some embodiments, the released transcriptional regulator modulates expression of a gene product of the cell. In some embodiments, the released transcriptional regulator modulates expression of a heterologous gene product in the cell. A heterologous gene product is one that is not normally produced by the cell. For example, the cell can be genetically modified with a nucleic acid comprising a nucleotide sequence encoding the heterologous gene product.

In some embodiments, the heterologous gene product is a secreted gene product. In some embodiments, the heterologous gene product is a cell surface gene product. In some cases, the heterologous gene product is a cytoplasmic gene product. In some embodiments, the released transcriptional regulator simultaneously modulates expression of two or more heterologous gene products in the cell.

In some embodiments, the heterologous gene product in the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen derived protein, a proliferation inducer, a receptor, an RNA guided nuclease, a site-specific nuclease, a T cell receptor (TCR), a chimeric antigen receptor (CAR), a toxin, a toxin-derived protein, a transcriptional regulator, a transcriptional activator, a transcriptional repressor, a translation regulator, a translational activator, a translational repressor, an activating immuno-receptor, an antibody, an apoptosis inhibitor, an apoptosis inducer, an engineered T-cell receptor, an immuno-activator, an immuno-inhibitor, and an inhibiting immuno-receptor.

In some embodiments, the released transcriptional regulator modulates differentiation of the cell, and wherein the cell is an immune cell, a stem cell, a progenitor cell, or a precursor cell.

The chimeric receptors of the disclosure provide a higher degree of expression than a standard SynNotch receptor, when using identical binding domains and ICDs. In some embodiments, depending on the ligand/binding domain pair and their affinity, the Notch receptor of the disclosure can provide expression enhancement of about 10%, about 15%, about 20%, about 25%, about 30%, about 35%, about 40%, about 45%, or about 50% higher than a corresponding SynNotch receptor.

Additionally, the chimeric receptors of the disclosure can provide transcriptional regulation that responds to the degree of T cell activation, independent of ligand binding. This permits additional flexibility in use, for example in cases where it is desired to enhance or suppress a T cell response when activated despite the absence of the chimeric receptor ligand.

### Systems and Kits

Also provided herein are systems and kits including the chimeric polypeptides, Notch receptors, recombinant nucleic acids, recombinant cells, or pharmaceutical compositions provided and described herein as well as written instructions for making and using the same. For example, provided herein, in some embodiments, are systems and/or kits that include one or more of: a chimeric polypeptide as described herein, a Notch receptor as described herein, a recombinant nucleic acids as described herein, a recombinant cell as described herein, or a pharmaceutical composition as described herein. In some embodiments, the systems and/or kits of the disclosure further include one or more syringes (including pre-filled syringes) and/or catheters (including pre-filled syringes) used to administer one any of the provided chimeric polypeptides, Notch receptors, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a health condition (e.g., disease) in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the chimeric receptor polypeptides.

In some embodiments, the components of a system or kit can be in separate containers. In some other embodiments, the components of a system or kit can be combined in a single container.

In some embodiments, a system or kit can further include instructions for using the components of the kit to practice the methods. The instructions for practicing the methods are generally recorded on a suitable recording medium. For example, the instructions can be printed on a substrate, such as paper or plastic, and the like. The instructions can be present in the kits as a package insert, in the labeling of the container of the kit or components thereof (i.e., associated with the packaging or sub-packaging), and the like. The instructions can be present as an electronic storage data file present on a suitable computer readable storage medium, e.g. CD-ROM, diskette, flash drive, and the like. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited above.

Additional embodiments are disclosed in further detail in the following examples, which are provided by way of illustration and are not in any way intended to limit the scope of this disclosure or the claims.

### Example 1

### Design and Construction of Chimeric Receptor and Response Element Constructs

This Example describes the design and construction of a family of chimeric Notch receptors. Detailed information for various exemplary receptors of the disclosure can be found in Tables 1 and 2 below.

TABLE 1
This table provides a brief description for each of the chimeric Notch receptors of the disclosure,
their corresponding transmembrane domains (TMD), as well as corresponding sequence identifiers
of the TMD sequences. The genes from which the transmembrane domains are derived from and
the corresponding accession numbers at the UniProt Knowledgebase (www.UniProt.org) are also
provided. SynNotch is a standard synthetic Notch receptor. MiniNotch: chimeric Notch receptor
which does not comprise a LIN-12-Notch repeat (LNR) and/or a heterodimerization domain (HD)
of a Notch receptor. Truncated CD8 HingeNotch is a chimeric Notch receptor including a truncated
hinge domain from CD8α incorporated between the ECD and TMD.

| | | | TMD SEQ |
|---|---|---|---|
| Plasmid ID | Receptor | Transmembrane Domain (TMD) | ID NO |
| pTMD001 | Human SynNotch | CLSTN1; Uniprot O94985 (AA860-880) | 1 |
| pTMD002 | Human SynNotch | CLSTN2; Uniprot Q9H4D0 (AA832-853) | 2 |
| pTMD003 | Human SynNotch | APLP1; Uniprot P51693 (AA581-603) | 3 |
| pTMD004 | Human SynNotch | APLP2; Uniprot Q06481 (AA693-716) | 4 |
| pTMD005 | Human SynNotch | LRP8; Uniprot Q14114 (AA827-848) | 5 |
| pTMD006 | Human SynNotch | APP; Uniprot P05067 (AA700-723) | 6 |
| pTMD007 | Human SynNotch | BTC; Uniprot P35070 (AA119-142) | 7 |
| pTMD008 | Human SynNotch | TGBR3; Uniprot Q03167 (AA788-810) | 8 |
| pTMD009 | Human SynNotch | SPN; Uniprot P16150 (AA254-277) | 9 |
| pTMD010 | Human SynNotch | CD44; Uniprot P16070 (AA650-672) | 10 |
| pTMD011 | Human SynNotch | CSF1R; Uniprot P07333 (AA518-538) | 11 |
| PTMD012 | Human SynNotch | CXCL16; Uniprot Q9H2A7 (AA206-227) | 12 |
| pTMD013 | Human SynNotch | CX3CL1; Uniprot P78423 (AA342-369) | 13 |
| pTMD014 | Human SynNotch | DCC; Uniprot P43146 (AA1098-1122) | 14 |
| pTMD015 | Human SynNotch | DLL1; Uniprot O00548 (AA546-569) | 15 |
| pTMD016 | Human SynNotch | DSG2; Uniprot Q14126 (AA610-635) | 16 |
| pTMD017 | Human SynNotch | DNER; Uniprot Q8NFT8 (AA641-662) | 17 |
| pTMD018 | Human SynNotch | DAG1; Uniprot Q14118 (AA750-775) | 18 |
| pTMD019 | Human SynNotch | CDH1; Uniprot P12830 (AA710-731) | 19 |
| pTMD020 | Human SynNotch | EPCAM; Uniprot P16422 (AA266-289) | 20 |
| pTMD021 | Human SynNotch | EPHA4; Uniprot P54764 (AA548-570) | 21 |
| pTMD022 | Human SynNotch | EPHB2; Uniprot P29323 (AA544-566) | 22 |
| pTMD023 | Human SynNotch | EFNB1; Uniprot P98172 (AA238-263) | 23 |
| pTMD024 | Human SynNotch | EFNB2; Uniprot P52799 (AA230-250) | 24 |
| pTMD025 | Human SynNotch | ErbB4; Uniprot Q15303 (AA652-675) | 25 |
| pTMD026 | Human SynNotch | GHR; Uniprot P10912 (AA265-288) | 26 |
| pTMD027 | Human SynNotch | HLA-A; Uniprot P01892 (AA309-332) | 27 |
| pTMD028 | Human SynNotch | IFNAR2; Uniprot P48551 (AA244-264) | 28 |
| pTMD029 | Human SynNotch | IGF1R; Uniprot P08069 (AA936-959) | 29 |
| pTMD030 | Human SynNotch | IL1R1; Uniprot P14778 (AA337-356) | 30 |
| pTMD031 | Human SynNotch | IL1R2; Uniprot P27930 (AA344-369) | 31 |
| pTMD032 | Human SynNotch | IL6R; Uniprot P08887 (AA366-386) | 32 |
| pTMD033 | Human SynNotch | INSR; Uniprot P06213 (AA957-979) | 33 |
| pTMD034 | Human SynNotch | ERN1; Uniprot O75460 (AA444-468) | 34 |
| pTMD035 | Human SynNotch | ERN2; Uniprot Q76MJ5 (AA431-451) | 35 |
| pTMD036 | Human SynNotch | JAG2; Uniprot Q9Y219 (AA1081-1106) | 36 |
| pTMD037 | Human SynNotch | KCNE1; Uniprot P15382 (AA44-66) | 37 |
| pTMD038 | Human SynNotch | KCNE2; Uniprot Q9Y6J6 (AA49-72) | 38 |
| pTMD039 | Human SynNotch | KCNE3; Uniprot Q9Y6H6 (AA58-80) | 39 |
| pTMD040 | Human SynNotch | KCNE4; Uniprot Q8WWG9 (AA87-109) | 40 |
| pTMD041 | Human SynNotch | KL; Uniprot Q9UEF7 (AA982-1004) | 41 |
| pTMD042 | Human SynNotch | CHL1; Uniprot O00533 (AA1083-1103) | 42 |
| pTMD043 | Human SynNotch | PTPRF; Uniprot P10586 (AA1264-1284) | 43 |
| pTMD044 | Human SynNotch | LRP1; Uniprot Q07954 (AA4420-4444) | 44 |
| pTMD045 | Human SynNotch | LRP1B; Uniprot Q9NZR2 (AA4445-4467) | 45 |
| pTMD046 | Human SynNotch | LRP2; Uniprot P98164 (AA4424-4446) | 46 |
| pTMD047 | Human SynNotch | LRP6; Uniprot O75581 (AA1371-1395) | 47 |
| pTMD048 | Human SynNotch | MUC1; Uniprot P15941 (AA1159-1186) | 48 |
| pTMD049 | Human SynNotch | CDH2; Uniprot P19022 (AA725-746) | 49 |
| pTMD050 | Human SynNotch | SCN1B; Uniprot Q07699 (AA161-182) | 50 |
| pTMD051 | Human SynNotch | SCN2B; Uniprot O60939 (AA160-180) | 51 |
| pTMD052 | Human SynNotch | SCN3B; Uniprot Q9NY72 (AA160-181) | 52 |
| pTMD053 | Human SynNotch | SCN4B; Uniprot Q8IWT1 (AA163-183) | 53 |
| pTMD054 | Human SynNotch | NECTIN1; Uniprot Q15223 (AA356-378) | 54 |
| pTMD055 | Human SynNotch | NRG1; Uniprot Q02297 (AA243-265) | 55 |
| pTMD056 | Human SynNotch | NRG2; Uniprot O14511 (AA406-428) | 56 |
| pTMD057 | Human SynNotch | NOTCH1; Uniprot P46531 (AA1736-1757) | 57 |
| pTMD058 | Human SynNotch | NOTCH2; Uniprot Q04721 (AA1678-1700) | 58 |
| pTMD059 | Human SynNotch | NOTCH3; Uniprot Q9UM47 (AA1644-1665) | 59 |
| pTMD060 | Human SynNotch | NOTCH4; Uniprot Q99466 (AA1448-1471) | 60 |
| pTMD061 | Human SynNotch | NPR3; Uniprot P17342 (AA482-504) | 61 |
| pTMD062 | Human SynNotch | Nradd; Uniprot Q8CJ26 (AA53-74) | 62 |
| pTMD063 | Human SynNotch | NGFR; Uniprot P08138 (AA251-272) | 63 |
| pTMD064 | Human SynNotch | PAM; Uniprot P19021 (AA864-887) | 64 |
| pTMD065 | Human SynNotch | PLXDC2; Uniprot Q6UX71 (AA455-478) | 65 |
| pTMD066 | Human SynNotch | PKHD1; Uniprot P08F94 (AA3859-3882) | 66 |
| pTMD067 | Human SynNotch | PCDHA4; Uniprot Q9UN74 (AA698-721) | 67 |
| pTMD068 | Human SynNotch | PCDHGC3; Uniprot Q9UN70 (AA694-715) | 68 |
| pTMD069 | Human SynNotch | PTPRZ1; Uniprot P23471 (AA1637-1662) | 69 |
| pTMD070 | Human SynNotch | AGER; Uniprot Q15109 (AA343-364) | 70 |
| pTMD071 | Human SynNotch | PTPRK; Uniprot Q15262 (AA753-774) | 71 |
| pTMD072 | Human SynNotch | PTPRM; Uniprot P28827 (AA743-764) | 72 |
| pTMD073 | Human SynNotch | ROBO1; Uniprot Q9Y6N7 (AA898-919) | 73 |
| pTMD074 | Human SynNotch | SORCS3; Uniprot Q9UPU3 (AA1126-1146) | 74 |
| pTMD075 | Human SynNotch | SORCS1; Uniprot Q8WY21 (AA1100-1120) | 75 |
| pTMD076 | Human SynNotch | SORL1; Uniprot Q92673 (AA2138-2160) | 76 |
| pTMD077 | Human SynNotch | SORT1; Uniprot Q99523 (AA756-778) | 77 |
| pTMD078 | Human SynNotch | SDC1; Uniprot P18827 (AA255-276) | 78 |
| pTMD079 | Human SynNotch | SDC2; Uniprot P34741 (AA145-169) | 79 |
| pTMD080 | Human SynNotch | SDC3; Uniprot O75056 (AA388-409) | 80 |
| pTMD081 | Human SynNotch | TIE1; Uniprot P35590 (AA760-786) | 81 |
| pTMD082 | Human SynNotch | TYR; Uniprot P14679 (AA477-500) | 82 |
| pTMD083 | Human SynNotch | TYRP1; Uniprot P17643 (AA478-501) | 83 |
| pTMD084 | Human SynNotch | DCT; Uniprot P40126 (AA473-495) | 84 |
| pTMD085 | Human SynNotch | VASN; Uniprot Q6EMK4 (AA576-599) | 85 |
| pTMD086 | Human SynNotch | CDH5; Uniprot P33151 (AA600-620) | 86 |
| pTMD087 | Human SynNotch | FLT1; Uniprot P17948 (AA759-780) | 87 |
| pTMD088 | Human SynNotch | VLDLR; Uniprot P98155 (AA798-819) | 88 |
| pTMD089 | Human SynNotch | Notch1_Danio rerio; Uniprot P46530 (AA1727-1748) | 89 |
| pTMD090 | Human SynNotch | Notch1_D. melanogaster; Uniprot P07207 (AA1746- | 90 |
| | | 1767) | |
| pTMD091 | Human SynNotch | Notch1_Xenopus laebis; Uniprot P21783 (AA1730-1750) | 91 |
| pTMD092 | Human SynNotch | Notch1_Gallus gallus; Uniprot F1NZ70 (AA1744-1766) | 92 |
| pTMD093 | Human SynNotch | Nicastrin; Uniprot Q92542 (AA670-692) | 93 |
| pTMD094 | Human SynNotch | CD147; Uniprot P35613 (AA324-346) | 94 |
| pTMD101 | Human miniNotch | CLSTN1; Uniprot O94985 (AA860-880) | 1 |
| pTMD102 | Human miniNotch | CLSTN2; Uniprot Q9H4D0 (AA832-853) | 2 |
| pTMD103 | Human miniNotch | APLP1; Uniprot P51693 (AA581-603) | 3 |
| pTMD104 | Human miniNotch | APLP2; Uniprot Q06481 (AA693-716) | 4 |
| pTMD105 | Human miniNotch | LRP8; Uniprot Q14114 (AA827-848) | 5 |
| pTMD106 | Human miniNotch | APP; Uniprot P05067 (AA700-723) | 6 |
| pTMD107 | Human miniNotch | BTC; Uniprot P35070 (AA119-142) | 7 |
| pTMD108 | Human miniNotch | TGBR3; Uniprot Q03167 (AA788-810) | 8 |
| pTMD109 | Human miniNotch | SPN; Uniprot P16150 (AA254-277) | 9 |
| pTMD110 | Human miniNotch | CD44; Uniprot P16070 (AA650-672) | 10 |
| pTMD111 | Human miniNotch | CSF1R; Uniprot P07333 (AA518-538) | 11 |
| pTMD112 | Human miniNotch | CXCL16; Uniprot Q9H2A7 (AA206-227) | 12 |
| pTMD113 | Human miniNotch | CX3CL1; Uniprot P78423 (AA342-369) | 13 |
| pTMD114 | Human miniNotch | DCC; Uniprot P43146 (AA1098-1122) | 14 |
| pTMD115 | Human miniNotch | DLL1; Uniprot O00548 (AA546-569) | 15 |
| pTMD116 | Human miniNotch | DSG2; Uniprot Q14126 (AA610-635) | 16 |
| pTMD117 | Human miniNotch | DNER; Uniprot Q8NFT8 (AA641-662) | 17 |
| pTMD118 | Human miniNotch | DAG1; Uniprot Q14118 (AA750-775) | 18 |
| pTMD119 | Human miniNotch | CDH1; Uniprot P12830 (AA710-731) | 19 |
| pTMD120 | Human miniNotch | EPCAM; Uniprot P16422 (AA266-289) | 20 |
| pTMD121 | Human miniNotch | EPHA4; Uniprot P54764 (AA548-570) | 21 |
| pTMD122 | Human miniNotch | EPHB2; Uniprot P29323 (AA544-566) | 22 |
| pTMD123 | Human miniNotch | EFNB1; Uniprot P98172 (AA238-263) | 23 |
| pTMD124 | Human miniNotch | EFNB2; Uniprot P52799 (AA230-250) | 24 |
| pTMD125 | Human miniNotch | ErbB4; Uniprot Q15303 (AA652-675) | 25 |
| pTMD126 | Human miniNotch | GHR; Uniprot P10912 (AA265-288) | 26 |
| pTMD127 | Human miniNotch | HLA-A; Uniprot P01892 (AA309-332) | 27 |
| pTMD128 | Human miniNotch | IFNAR2; Uniprot P48551 (AA244-264) | 28 |
| pTMD129 | Human miniNotch | IGF1R; Uniprot P08069 (AA936-959) | 29 |
| pTMD130 | Human miniNotch | IL1R1; Uniprot P14778 (AA337-356) | 30 |
| pTMD131 | Human miniNotch | IL1R2; Uniprot P27930 (AA344-369) | 31 |
| pTMD132 | Human miniNotch | IL6R; Uniprot P08887 (AA366-386) | 32 |
| pTMD133 | Human miniNotch | INSR; Uniprot P06213 (AA957-979) | 33 |
| pTMD134 | Human miniNotch | ERN1; Uniprot O75460 (AA444-468) | 34 |
| pTMD135 | Human miniNotch | ERN2; Uniprot Q76MJ5 (AA431-451) | 35 |
| pTMD136 | Human miniNotch | JAG2; Uniprot Q9Y219 (AA1081-1106) | 36 |
| pTMD137 | Human miniNotch | KCNE1; Uniprot P15382 (AA44-66) | 37 |
| pTMD138 | Human miniNotch | KCNE2; Uniprot Q9Y6J6 (AA49-72) | 38 |
| pTMD139 | Human miniNotch | KCNE3; Uniprot Q9Y6H6 (AA58-80) | 39 |
| pTMD140 | Human miniNotch | KCNE4; Uniprot Q8WWG9 (AA87-109) | 40 |
| pTMD141 | Human miniNotch | KL; Uniprot Q9UEF7 (AA982-1004) | 41 |
| pTMD142 | Human miniNotch | CHL1; Uniprot O00533 (AA1083-1103) | 42 |
| pTMD143 | Human miniNotch | PTPRF; Uniprot P10586 (AA1264-1284) | 43 |
| pTMD144 | Human miniNotch | LRP1; Uniprot Q07954 (AA4420-4444) | 44 |
| pTMD145 | Human miniNotch | LRP1B; Uniprot Q9NZR2 (AA4445-4467) | 45 |
| pTMD146 | Human miniNotch | LRP2; Uniprot P98164 (AA4424-4446) | 46 |
| pTMD147 | Human miniNotch | LRP6; Uniprot O75581 (AA1371-1395) | 47 |
| pTMD148 | Human miniNotch | MUC1; Uniprot P15941 (AA1159-1186) | 48 |
| pTMD149 | Human miniNotch | CDH2; Uniprot P19022 (AA725-746) | 49 |
| pTMD150 | Human miniNotch | SCN1B; Uniprot Q07699 (AA161-182) | 50 |
| pTMD151 | Human miniNotch | SCN2B; Uniprot O60939 (AA160-180) | 51 |
| pTMD152 | Human miniNotch | SCN3B; Uniprot Q9NY72 (AA160-181) | 52 |
| pTMD153 | Human miniNotch | SCN4B; Uniprot Q8IWT1 (AA163-183) | 53 |
| pTMD154 | Human miniNotch | NECTIN1; Uniprot Q15223 (AA356-378) | 54 |
| pTMD155 | Human miniNotch | NRG1; Uniprot Q02297 (AA243-265) | 55 |
| pTMD156 | Human miniNotch | NRG2; Uniprot O14511 (AA406-428) | 56 |
| pTMD157 | Human miniNotch | NOTCH1; Uniprot P46531 (AA1736-1757) | 57 |
| pTMD158 | Human miniNotch | NOTCH2; Uniprot Q04721 (AA1678-1700) | 58 |
| pTMD159 | Human miniNotch | NOTCH3; Uniprot Q9UM47 (AA1644-1665) | 59 |
| pTMD160 | Human miniNotch | NOTCH4; Uniprot Q99466 (AA1448-1471) | 60 |
| pTMD161 | Human miniNotch | NPR3; Uniprot P17342 (AA482-504) | 61 |
| pTMD162 | Human miniNotch | Nradd; Uniprot Q8CJ26 (AA53-74) | 62 |
| pTMD163 | Human miniNotch | NGFR; Uniprot P08138 (AA251-272) | 63 |
| pTMD164 | Human miniNotch | PAM; Uniprot P19021 (AA864-887) | 64 |
| pTMD165 | Human miniNotch | PLXDC2; Uniprot Q6UX71 (AA455-478) | 65 |
| pTMD166 | Human miniNotch | PKHD1; Uniprot P08F94 (AA3859-3882) | 66 |
| pTMD167 | Human miniNotch | PCDHA4; Uniprot Q9UN74 (AA698-721) | 67 |
| pTMD168 | Human miniNotch | PCDHGC3; Uniprot Q9UN70 (AA694-715) | 68 |
| pTMD169 | Human miniNotch | PTPRZ1; Uniprot P23471 (AA1637-1662) | 69 |
| pTMD170 | Human miniNotch | AGER; Uniprot Q15109 (AA343-364) | 70 |
| pTMD171 | Human miniNotch | PTPRK; Uniprot Q15262 (AA753-774) | 71 |
| pTMD172 | Human miniNotch | PTPRM; Uniprot P28827 (AA743-764) | 72 |
| pTMD173 | Human miniNotch | ROBO1; Uniprot Q9Y6N7 (AA898-919) | 73 |
| pTMD174 | Human miniNotch | SORCS3; Uniprot Q9UPU3 (AA1126-1146) | 74 |
| pTMD175 | Human miniNotch | SORCS1; Uniprot Q8WY21 (AA1100-1120) | 75 |
| pTMD176 | Human miniNotch | SORL1; Uniprot Q92673 (AA2138-2160) | 76 |
| pTMD177 | Human miniNotch | SORT1; Uniprot Q99523 (AA756-778) | 77 |
| pTMD178 | Human miniNotch | SDC1; Uniprot P18827 (AA255-276) | 78 |
| pTMD179 | Human miniNotch | SDC2; Uniprot P34741 (AA145-169) | 79 |
| pTMD180 | Human miniNotch | SDC3; Uniprot O75056 (AA388-409) | 80 |
| pTMD181 | Human miniNotch | TIE1; Uniprot P35590 (AA760-786) | 81 |
| pTMD182 | Human miniNotch | TYR; Uniprot P14679 (AA477-500) | 82 |
| pTMD183 | Human miniNotch | TYRP1; Uniprot P17643 (AA478-501) | 83 |
| pTMD184 | Human miniNotch | DCT; Uniprot P40126 (AA473-495) | 84 |
| pTMD185 | Human miniNotch | VASN; Uniprot Q6EMK4 (AA576-599) | 85 |
| pTMD186 | Human miniNotch | CDH5; Uniprot P33151 (AA600-620) | 86 |
| pTMD187 | Human miniNotch | FLT1; Uniprot P17948 (AA759-780) | 87 |
| pTMD188 | Human miniNotch | VLDLR; Uniprot P98155 (AA798-819) | 88 |
| pTMD189 | Human miniNotch | Notch1_Daniorerio; Uniprot P46530 (AA1727-1748) | 89 |
| pTMD190 | Human miniNotch | Notch1_D. melanogaster; Uniprot P07207 (AA1746- | 90 |
| | | 1767) | |
| pTMD191 | Human miniNotch | Notch1_Xenopus laebis; Uniprot P21783 (AA1730-1750) | 91 |
| pTMD192 | Human miniNotch | Notch1_Gallus gallus; Uniprot F1NZ70 (AA1744-1766) | 92 |
| pTMD193 | Human miniNotch | Nicastrin; Uniprot Q92542 (AA670-692) | 93 |
| pTMD194 | Human miniNotch | CD147; Uniprot P35613 (AA324-346) | 94 |
| pTMD201 | Human truncated | CLSTN1; Uniprot O94985 (AA860-880) | 1 |
| | CD8 HingeNotch | | |
| pTMD202 | Human truncated | CLSTN2; Uniprot Q9H4D0 (AA832-853) | 2 |
| | CD8 HingeNotch | | |

TABLE 2
This table provides a brief description for each of the chimeric Notch receptors and the respective components
(with components separated by commas). Unless otherwise noted, the entry refers to a protein of human origin.

| Construct | | | | | | |
|---|---|---|---|---|---|---|
| ID | Receptor Description | ECD | N-JMD | TMD | STS | TF |
| pTMD001- | Human SynNotch with | CD8α signal peptide, | Notch1 | See Table 1 | Notch1 | Gal4, |
| pTMD094 | swapped TMD | myc-tag, anti-CD19 scFv | | | | VP64 |
| pTMD101- | Human miniNotch | CD8α signal peptide, | Notch1, | See Table 1 | Notch1 | Gal4, |
| pTMD194 | with swapped TMD | myc-tag, anti-CD19 scFv | Notch1 | | | VP64 |
| pTMD201 | Human truncated CD8 | CD8α signal peptide, | truncated | See Table 1 | Notch1 | Gal4, |
| and | HingeNotch with | myc-tag, anti-CD19 scFv | CD8 | | | VP64 |
| pTMD202 | swapped TMD | | hinge | | | |
| pIZ341 | anti-CD19 scFv | CD8a signal peptide, | CD8 | Notch1 | Notch1 | Gal4, |
| | connected to | myc-tag, anti-CD19 scFv | hinge | | | VP64 |
| | hsNotch1TMD_Gal4V | | | | | |
| | P64 with full CD8 | | | | | |
| | hinge | | | | | |
| pIZ343 | anti-CD19 scFv | CD8a signal peptide, | truncated | Notch1 | Notch1 | Gal4, |
| | connected to | myc-tag, anti-CD19 scFv | CD8 | | | VP64 |
| | hsNotch1TMD_Gal4V | | hinge | | | |
| | P64 with truncated | | | | | |
| | CD8 hinge, one | | | | | |
| | cysteine | | | | | |
| pIZ358 | anti-CD19 scFv | CD8a signal peptide, | CD28 | Notch1 | Notch1 | Gal4, |
| | connected to | myc-tag, anti-CD19 scFv | hinge | | | VP64 |
| | hsNotch1TMD_Gal4V | | | | | |
| | P64 with CD28 Hinge | | | | | |
| pIZ359 | anti-CD19 scFv | CD8a signal peptide, | (GGGGS) | Notch1 | Notch1 | Gal4, |
| | connected to | myc-tag, anti-CD19 scFv | 3 IgG4 | | | VP64 |
| | hsNotch1TMD_Gal4V | | hinge | | | |
| | P64 with IgG4 Hinge | | | | | |
| pIZ360 | anti-CD19 scFv | CD8a signal peptide, | OX40 | Notch1 | Notch1 | Gal4, |
| | connected to | myc-tag, anti-CD19 scFv | hinge | | | VP64 |
| | hsNotch1TMD_Gal4V | | | | | |
| | P64 with OX40 | | | | | |
| | trimeric hinge | | | | | |
| pIZ361 | pIZ343 with human | CD8a signal peptide, | truncated | Notch1 | Notch2 | Gal4, |
| | Notch1 STS replaced | myc-tag, anti-CD19 scFv | CD8 | | | VP64 |
| | by Notch2 STS | | hinge | | | |
| pIZ343FYIA | anti-ALPPL2 | CD8a signal peptide, | truncated | Notch1 | Notch1 | Gal4, |
| | scFv(FYIA) connected | myc-tag, anti-ALPPL2 | CD8 | | | VP64 |
| | to | scFv | hinge | | | |
| | hsNotch1TMD_Gal4V | | | | | |
| | P64 with truncated | | | | | |
| | CD8 hinge, one | | | | | |
| | cysteine | | | | | |
| pIZ343eGFP | pIZ343 with GFP | Mouse IgKVIII signal | truncated | Notch1 | Notch1 | Gal4, |
| | extracellular domain | peptide, eGFP | CD8 | | | VP64 |
| | | | hinge | | | |
| pIZ342 | anti-CD19 scFv | CD8a signal peptide, | truncated | Notch1 | Notch1 | Gal4, |
| | connected to | myc-tag, anti-CD19 scFv | CD8 | | | VP64 |
| | hsNotch1TMD_Gal4V | | hinge | | | |
| | P64 with truncated | | | | | |
| | CD8 hinge, no | | | | | |
| | cysteines | | | | | |
| pIZ362 | anti-CD19 scFv | CD8a signal peptide, | N- | Notch1 | Notch1 | Gal4, |
| | connected to | myc-tag, anti-CD19 scFv | truncated | | | VP64 |
| | hsNotch1TMD_Gal4V | | CD8Hinge | | | |
| | P64 with N-truncated | | | | | |
| | CD8 hinge, one | | | | | |
| | cysteine | | | | | |
| pIZ363 | anti-CD19 scFv | CD8a signal peptide, | N- | Notch1 | Notch1 | Gal4, |
| | connected to | myc-tag, anti-CD19 scFv | truncated | | | VP64 |
| | hsNotch1TMD_Gal4V | | CD8Hinge | | | |
| | P64 with N-truncated | | | | | |
| | CD8 hinge, two | | | | | |
| | cysteines | | | | | |
| pIZ361FYIA | anti-ALPPL2 | CD8a signal peptide, | truncated | Notch1 | Notch2 | Gal4, |
| | scFv(FYIA) connected | myc-tag, anti-ALPPL2 | CD8 | | | VP64 |
| | to | scFv | hinge | | | |
| | hsNotch1TMD_Gal4V | | | | | |
| | P64 with truncated | | | | | |
| | CD8 hinge, one | | | | | |
| | cysteine, Notch2 STS | | | | | |
| pIZ343BCMA | anti-BCMA scFV | CD8a signal peptide, | truncated | Notch1 | Notch1 | Gal4, |
| | connected to | myc-tag, anti-BCMA | CD8 | | | VP64 |
| | hsNotch1TMD_Gal4V | scFv | hinge | | | |
| | P64 with truncated | | | | | |
| | CD8 hinge, one | | | | | |
| | cysteine | | | | | |
| pIZ361BCMA | anti-BCMA scFV | CD8a signal peptide, | truncated | Notch1 | Notch2 | Gal4, |
| | connected to | myc-tag, anti-BCMA | CD8 | | | VP64 |
| | hsNotch1TMD_Gal4V | scFv | hinge | | | |
| | P64 with truncated | | | | | |
| | CD8 hinge, one | | | | | |
| | cysteine, Notch2 STS | | | | | |
| pIZ343(4D5-8) | anti-Her2 scFV (4D5- | CD8a signal peptide, | truncated | Notch1 | Notch1 | Gal4, |
| | 8) connected to | myc-tag, anti-HER2 scFv | CD8 | | | VP64 |
| | hsNotch1TMD_Gal4V | | hinge | | | |
| | P64 with truncated | | | | | |
| | CD8 hinge, one | | | | | |
| | cysteine | | | | | |
| pIZ361(4D5-8) | anti-Her2 scFV (4D5- | CD8a signal peptide, | truncated | Notch1 | Notch2 | Gal4, |
| | 8) connected to | myc-tag, anti-HER2 scFv | CD8 | | | VP64 |
| | hsNotch1TMD_Gal4V | | hinge | | | |
| | P64 with truncated | | | | | |
| | CD8 hinge, one | | | | | |
| | cysteine, Notch2 STS | | | | | |
| pIZ343(4D5-7) | anti-Her2 scFV (4D5- | CD8a signal peptide, | truncated | Notch1 | Notch1 | Gal4, |
| | 7) connected to | myc-tag, anti-HER2 scFv | CD8 | | | VP64 |
| | hsNotch1TMD_Gal4V | | hinge | | | |
| | P64 with truncated | | | | | |
| | CD8 hinge, one | | | | | |
| | cysteine | | | | | |
| pIZ361(4D5-7) | anti-Her2 scFV (4D5- | CD8a signal peptide, | truncated | Notch1 | Notch2 | Gal4, |
| | 7) connected to | myc-tag, anti-HER2 scFv | CD8 | | | VP64 |
| | hsNotch1TMD_Gal4V | | hinge | | | |
| | P64 with truncated | | | | | |
| | CD8 hinge, one | | | | | |
| | cysteine, Notch2 STS | | | | | |
| pRay068A | anti-BCMA fully | CD8a signal peptide, anti- | truncated | Notch1 | Notch2 | Gal4, |
| | humanized VH | BCMA VH domain | CD8 | | | VP64 |
| | domain connected to | | hinge | | | |
| | hsNotch1TMD_Gal4V | | | | | |
| | P64 with truncated | | | | | |
| | CD8 hinge, one | | | | | |
| | cysteine, Notch2 STS | | | | | |
| pRay068B | anti-BCMA fully | CD8a signal peptide, anti- | truncated | Notch1 | Notch2 | Gal4, |
| | humanized VH | BCMA VH domain | CD8 | | | VP64 |
| | domain connected to | | hinge | | | |
| | hsNotch1TMD_Gal4V | | | | | |
| | P64 with truncated | | | | | |
| | CD8 hinge, one | | | | | |
| | cysteine, Notch2 STS | | | | | |
| pIZ370 | antiCD19scFv- | CD8a signal peptide, | truncated | CLSTN1 | CLSTN1 | Gal4, |
| | CD8Hinge2- | myc-tag, anti-CD19 scFv | CD8 | | | VP64 |
| | CLSTN1TMD- | | hinge | | | |
| | CLSTN1STS- | | | | | |
| | Gal4VP64 | | | | | |
| pIZ371 | antiCD19scFv- | CD8a signal peptide, | truncated | CLSTN2 | CLSTN2 | Gal4, |
| | CD8Hinge2- | myc-tag, anti-CD19 scFv | CD8 | | | VP64 |
| | CLSTN2TMD- | | hinge | | | |
| | CLSTN2STS- | | | | | |
| | Gal4VP64 | | | | | |
| pTMD201 | antiCD19scFv- | CD8a signal peptide, | truncated | CLSTN1 | Notch1 | Gal4, |
| | CD8Hinge2- | myc-tag, anti-CD19 scFv | CD8 | | | VP64 |
| | CLSTN1TMD- | | hinge | | | |
| | Notch1STS-Gal4VP64 | | | | | |
| pTMD202 | antiCD19scFv- | CD8a signal peptide, | truncated | CLSTN2 | Notch1 | Gal4, |
| | CD8Hinge2- | myc-tag, anti-CD19 scFv | CD8 | | | VP64 |
| | CLSTN2TMD- | | hinge | | | |
| | Notch1STS-Gal4VP64 | | | | | |

The chimeric receptors described in Tables 1-2 above were built by fusing a single-chain antigen-binding fragment CD 19 scFv (Porter D L et al., 2011) to the corresponding receptor scaffold and a synthetic transcriptional regulator GAL4-VP64. For the construction of these receptors, DNA fragments coding for the amino acid sequences provided in Table 1 and Sequence Listing were PCR amplified from synthesized gene fragments or plasmids containing DNA sequence for the indicated protein, and assembled using standard cloning techniques (e.g., overhang PCR, fusion PCR, and In-fusion cloning) with flanking translation start and stop sequences, into a BamHI cloning site of the lentiviral expression vector pHR-SIN-pGK.

The transcriptional regulator GAL4-VP64 used in these experiments contained a DNA domain from yeast GAL4 transcription factor fused to an activation domain VP64, which consists of a tetrameric repeat of the minimal activation domain (amino acids 437-447) of the herpes simplex protein VP16. All receptors contained an N-terminal CD8α signal peptide (MALPVTALLLPLALLLHAARP) (SEQ ID NO: 100) for membrane targeting and a myc-tag (EQKLISEEDL) (SEQ ID NO: 101) for convenient determination of surface expression with an antibody conjugated to a fluorescent dye (α-myc A647®, Cell Signaling Technology, Cat #2233). The receptors were each cloned into a modified lentiviral pHR′ SIN:CSW vector (K. T. Roybal et al., Cell (2016) 167(2):419-32) containing a phosphoglycerate kinase (PGK) promoter for all primary T cell experiments described in Examples 3-4 below.

The pHR′ SIN:CSW vector was also modified to produce the response element plasmids. For this purpose, five copies of a target sequence for binding of GAL4 DBD domain (GGAGCACTGTCCTCCGAACG) (SEQ ID NO: 102) were cloned 5′ to a minimal pybTATA promoter. Also included in the response element plasmids is a PGK promoter that constitutively drives expression of a yellow fluorescent reporter protein (mCitrine) to conveniently identify successfully transduced T cells.

For the construction of all inducible BFP vectors, the coding sequence for a blue fluorescent reporter protein (BFP) was cloned via a BamHI site in the multiple cloning site located 3′ to the GAL4 response elements. For the construction of all inducible CAR vectors, the CARs were tagged c-terminally with a green fluorescent reporter protein (GFP) and were cloned via a BamHI site in the multiple cloning site located 3′ to the GAL4 response elements. All constructs were cloned via cloning kit (In-Fusion® cloning, Clontech #ST0345) according to the manufacturer's instructions.

### Example 2

### Primary Human T-Cell Isolation and Culture

This Example describes the isolation and culture of primary human T cells that were subsequently used in various cell transduction experiments described in Example 3 below.

In these experiments, primary CD4⁺ and CD8⁺ T cells were isolated from blood after apheresis and enriched by negative selection using human T-cell isolation kits (human CD4⁺ or CD8⁺ enrichment cocktail; STEMCELL Technologies Cat #15062 and 15063). Blood was obtained from Blood Centers of the Pacific (San Francisco, Calif.) as approved by the University Institutional Review Board. T cells were cryopreserved in growth medium (RPMI-1640, UCSF cell culture core) with 20% human AB serum (Valley Biomedical Inc., #HP1022) and 10% DMSO. After thawing, T cells were cultured in human T cell medium containing X-VIVO™ 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments.

### Example 3

### Human T Cells were Stably Transduced with Lentiviral Vectors

The Example describes a general protocol used for lentiviral transduction of human T cells.

Generally, lentiviral vectors pseudo-typed with vesicular stomatitis virus envelope G protein (VSV-G) (pantropic vectors) were produced via transfection of Lenti-X™ 293T cells (Clontech #11131D) with a pHR′ SIN:CSW transgene expression vector and the viral packaging plasmids pCMVdR8.91 and pMD2.G using Mirus TransIT®-Lenti (Mirus, #MIR 6606). Generally, primary T cells were thawed the same day and, after 24 hours in culture, were stimulated with beads having anti-CD3 and anti-CD28 antibodies bound to the surface (Human T-Activator CD3/CD28 Dynabeads®, Life Technologies #11131D) at a 1:3 cell:bead ratio. At 48 hours, viral supernatant was harvested and the primary T cells were exposed to the virus for 24 hours. At Day 5 post T-cell stimulation, the beads were removed, and the T cells expanded until Day 14 when they were rested and could be used in assays. T cells were sorted for assays with a Beckton Dickinson (BD Biosciences) FACSAria™ II flow cytometer. AND-gate T cells exhibiting basal CAR expression were gated out during sorting.

### Example 4

### Stimulation of Primary T Cells In Vitro

This Example describes experiments performed to demonstrate the stimulation of primary T cells in vitro by the chimeric Notch polypeptides described herein.

For all in vitro T-cell stimulations, 1×10⁵ T cells were co-cultured with sender cells at a 1:1 ratio in flat bottom 96-well tissue culture plates. The cultures were analyzed at 24 hours for reporter activation with a BD Fortessa™ X-50. All flow cytometry analysis was performed in FlowJo™ software (TreeStar, Inc.).

### Example 5

This Example describes the generation of myelogenous leukemia “sender” cells expressing CD19 at equivalent levels as Daudi tumors.

The cancer cell lines used were K562 myelogenous leukemia cells (ATCC #CCL-243) and Daudi B cell lymphoblasts (ATCC #CCL-213). The K562 cells were lentivirally transduced to stably express human CD19 at equivalent levels as Daudi tumors. CD19 levels were determined by staining the cells with α-CD19 APC (Biolegend #302212). All cell lines were sorted for expression of the transgenes.

### Example 6

### Generation of Reporter Jurkat T Cells

This Example describes the generation of reporter Jurkat T cells that were subsequent used for the screening of transmembrane domains (TMD) and/or stop-transfer sequences (STS).

In these experiments, E6-1 Jurkat T cells (ATCC #TIB-152) were lentivirally transduced with a reporter plasmid carrying an inducible BFP reporter gene and a constitutive mCitrine reporter gene, as described previously (K. T. Roybal et al., Cell, 164:1-10, 2016). Reporter-positive Jurkat cells were sorted for mCitrine expression using a Beckton Dickinson (BD Biosciences) FACSAria™ II flow cytometer and expanded.

Lentiviral particles were produced with the receptor transgene expression vector as described previously (L. Morsut et al., Cell (2016) 164:780-91). Reporter-positive Jurkat cells were transduced with individual receptors and expanded for experimentation in 96 well plates.

### Example 7

This Example describes experiments performed to demonstrate the stimulation of Jurkat T cells in vitro by the chimeric Notch polypeptides described herein.

For all in vitro Jurkat T-cell stimulations, 1×10⁵ Jurkat T cells were co-cultured with sender cells at a 1:1 ratio in flat bottom 96-well tissue culture plates. The cultures were analyzed at 24 hours for receptor (myc) expression and reporter activation with a BD Fortessa X-50™. All flow cytometry analysis was performed in FlowJo™ software (TreeStar, Inc.). Receptors with positive TMD and STS hits, along with a selection of negative hits, were confirmed in human primary T-cells using the above protocols.

### Example 8

This Example describes experiments performed to the effect of substituting a heterologous transmembrane domain in three different chimeric Notch receptors, as determined by expression levels of a BFP reporter gene placed under control of the resulting chimeric Notch receptors.

In these experiments, amino acid sequences corresponding to transmembrane domains from 88 known human γ secretase target (Haapalaso, J Alzheimers Dis (2011) 25(1):3-28) and Notch family members from model organisms were incorporated into three different chimeric Notch receptors (SynNotch, MiniNotch, and HingeNotch).

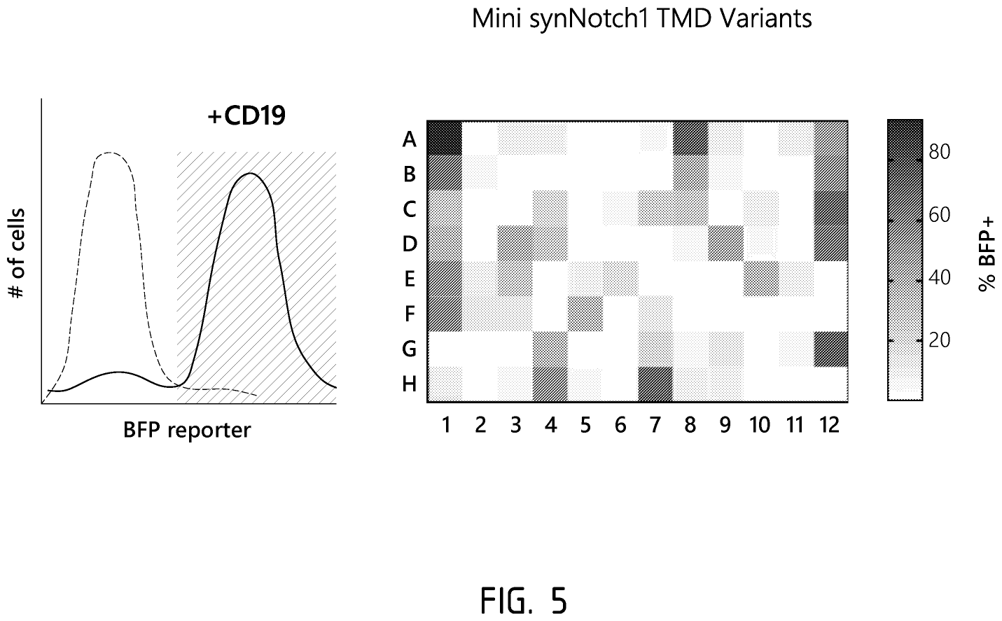

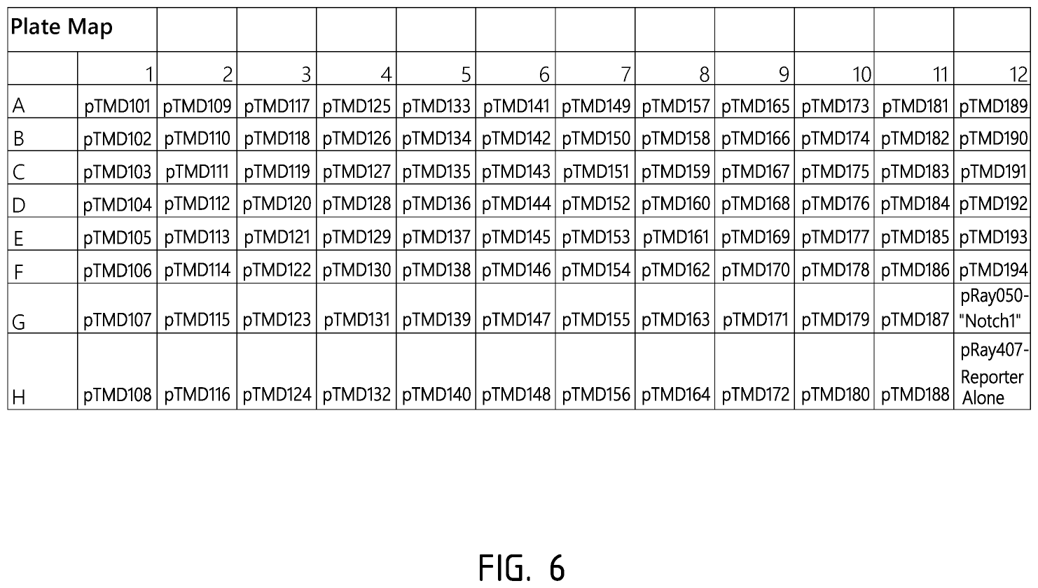

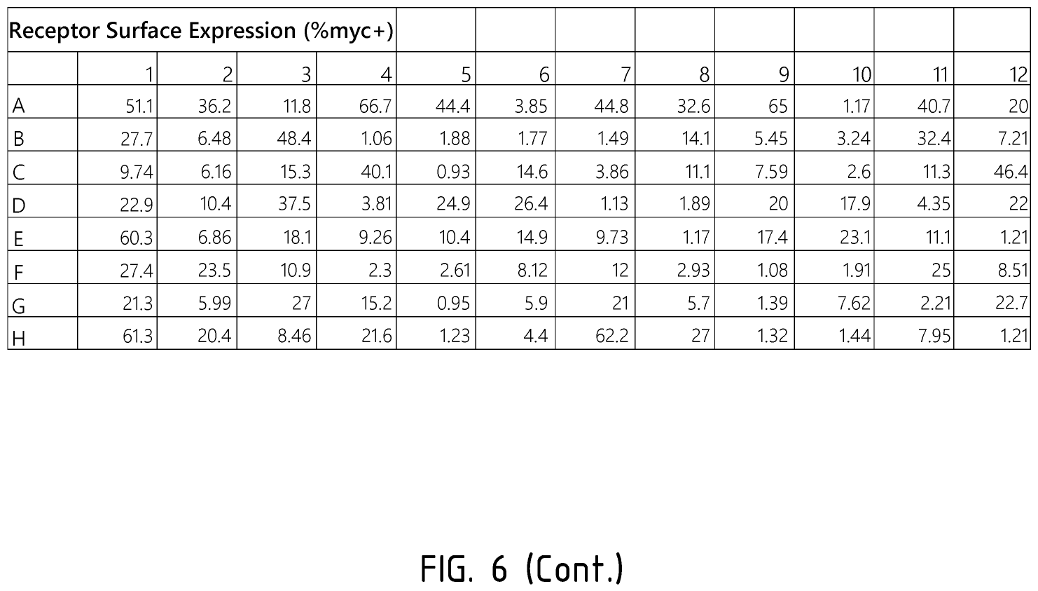

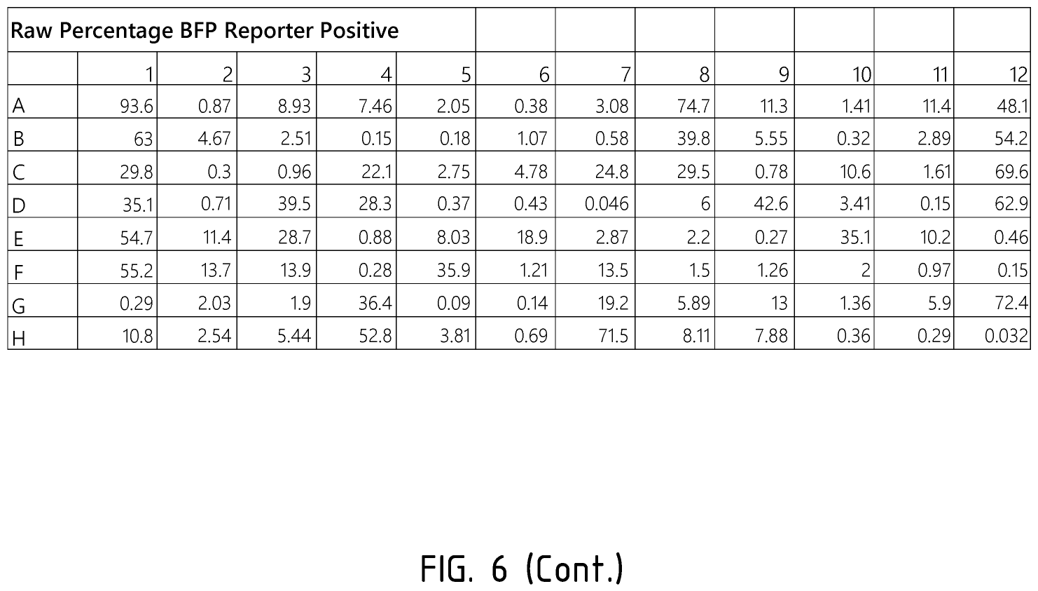

The initial screen was perform in Jurkat cells, and candidates identified from the initial screen (hits) were further validated in assays performed with T lymphocytes. The results are shown in FIG. 5.

### Example 9

This Example describes experiments performed to show that TMD regulates Notch activation.

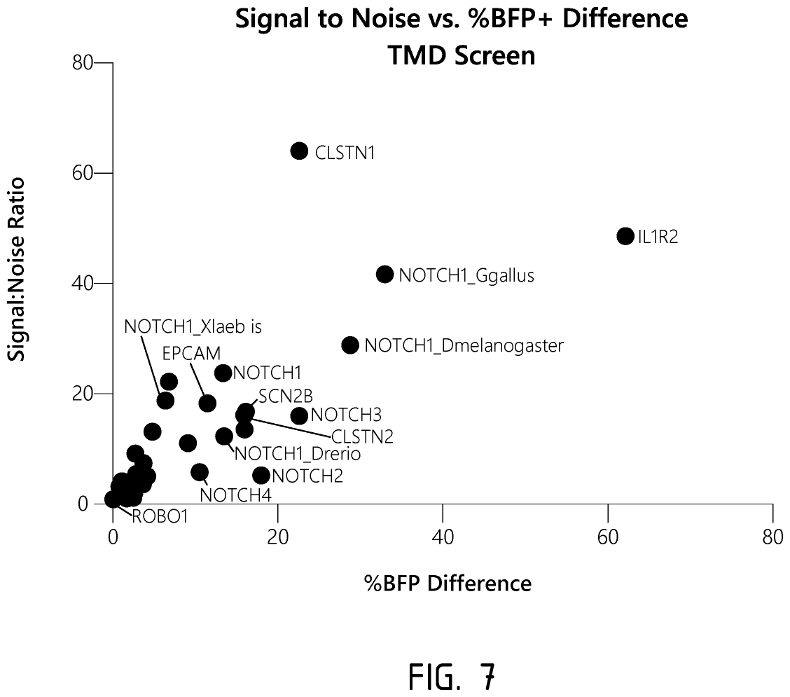

As shown in FIG. 7, Jurkat T cells expressing a BFP reporter construct were transduced with lentiviral constructs containing Notch receptors with TMD variants. Jurkats were co-cultured 1:1 with control CD19(−) or CD19(+) K562 cells. BFP reporter gene activation was subsequently measured using a Fortessa X-50 (BD Biosciences). Signal to noise ratios from the MFIs of BFP+ cells under CD19+K562 versus K562 conditions are plotted against the change in MFI in the two conditions.

### Example 10

This Example describes mutational analysis of the Notch1 transmembrane domain (TMD) in Hinge-Notch constructs.

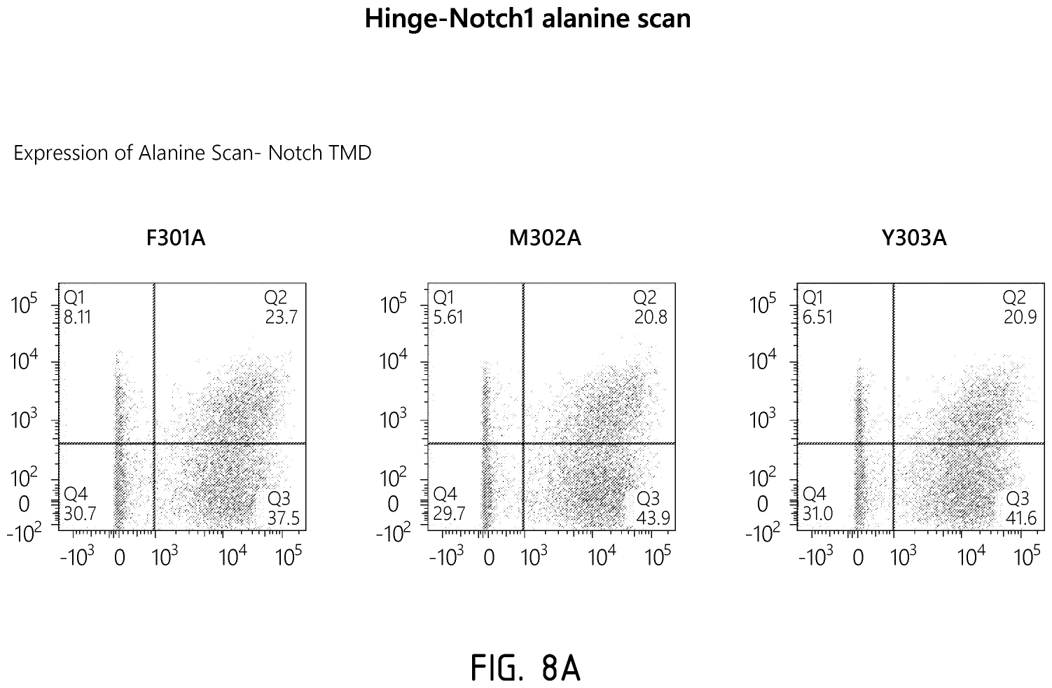

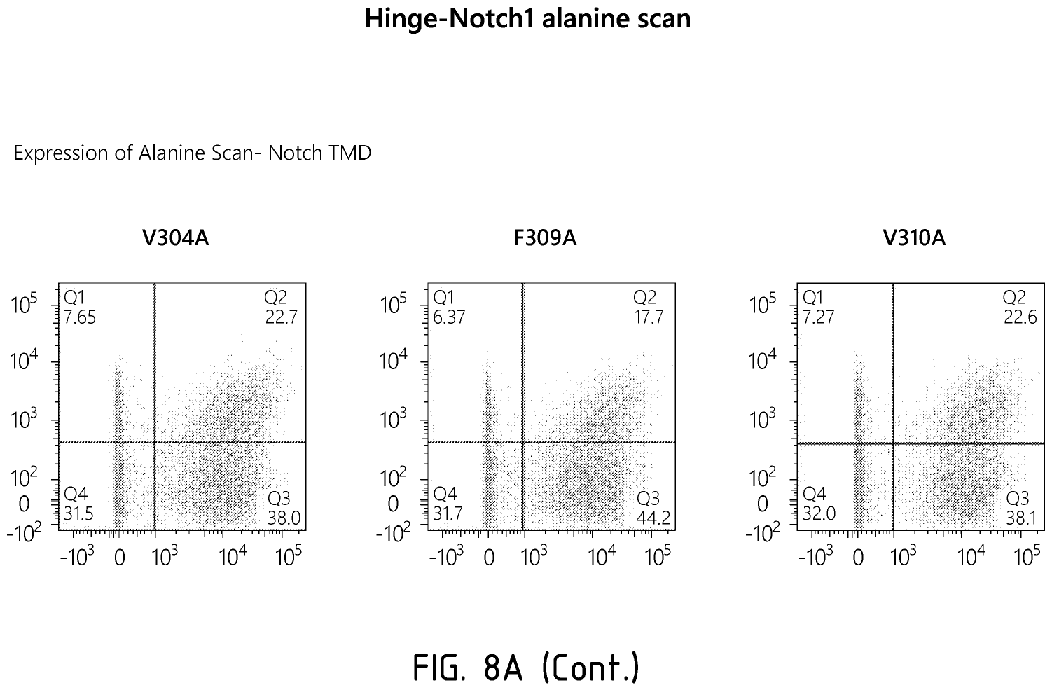

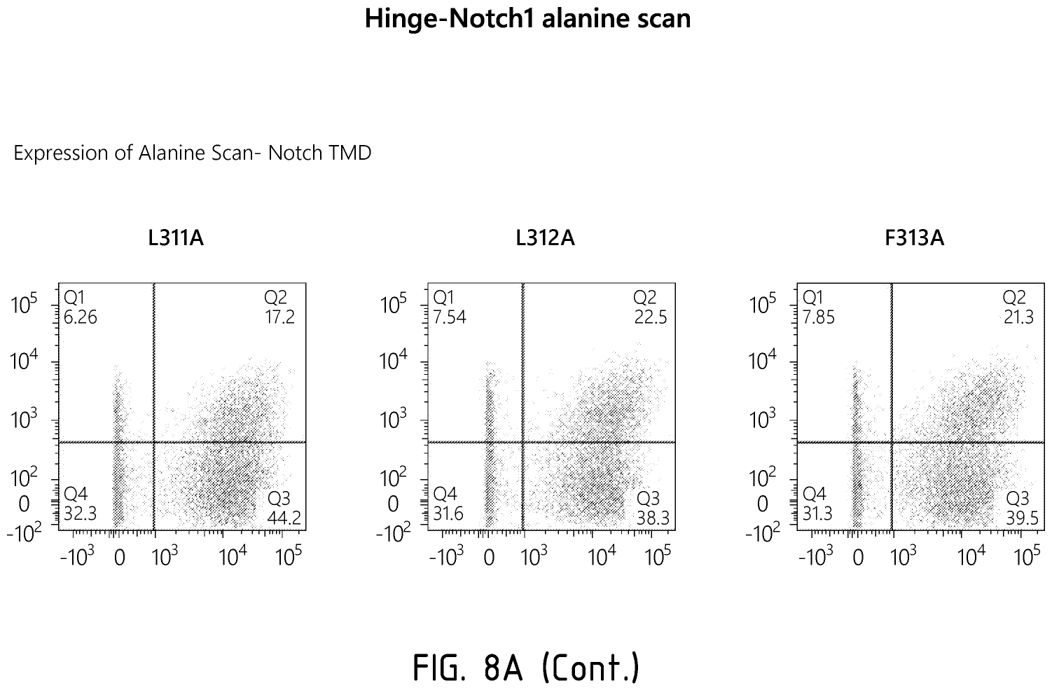

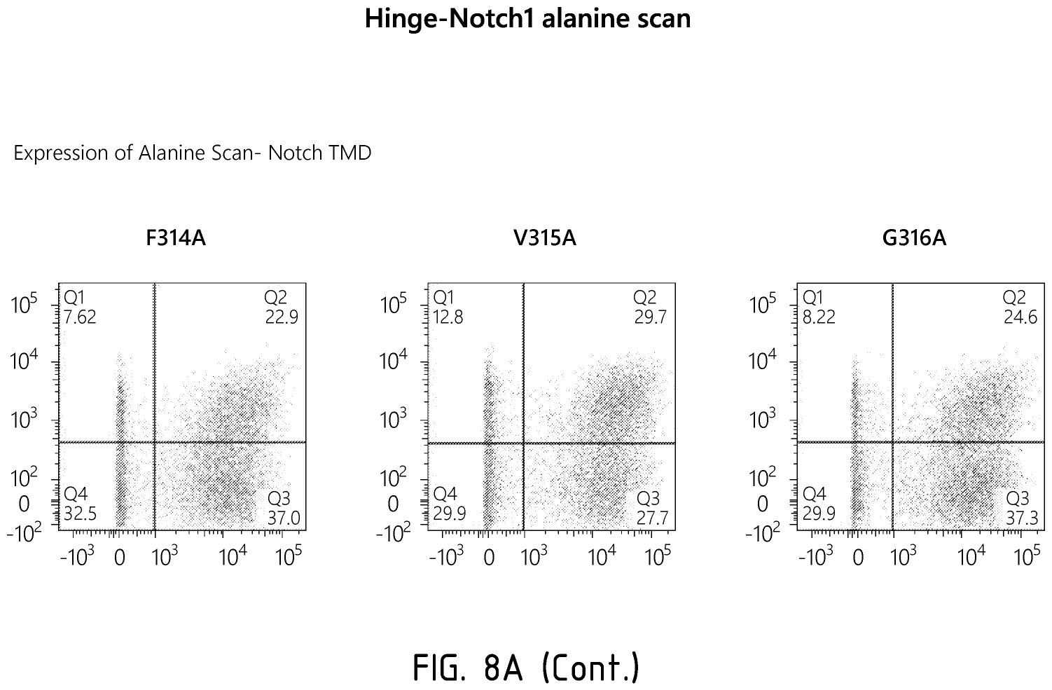

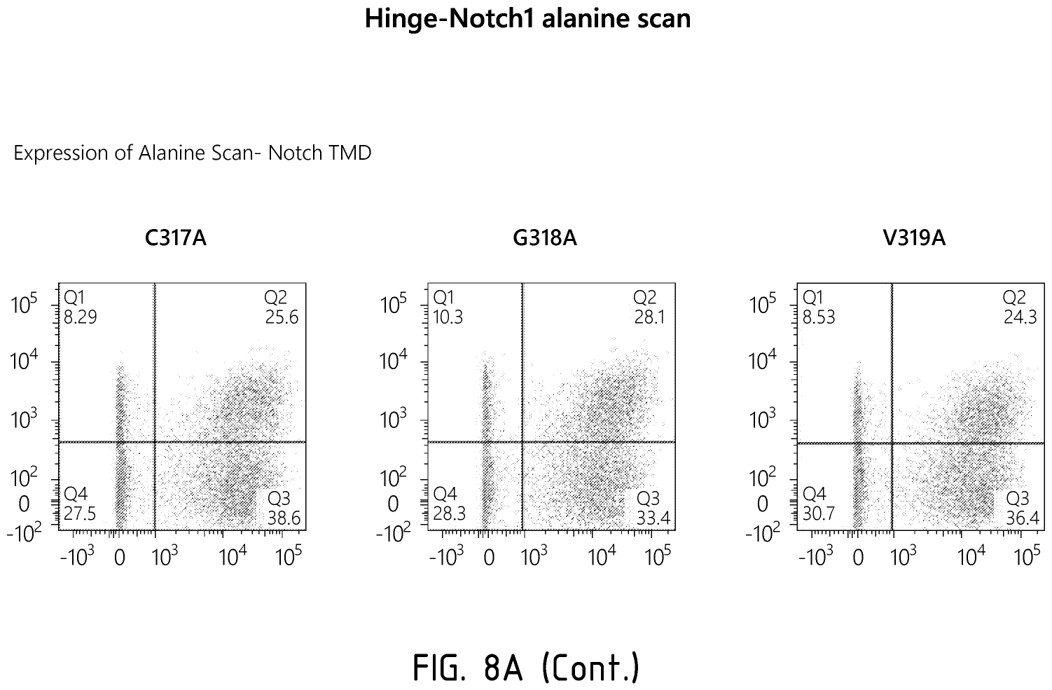

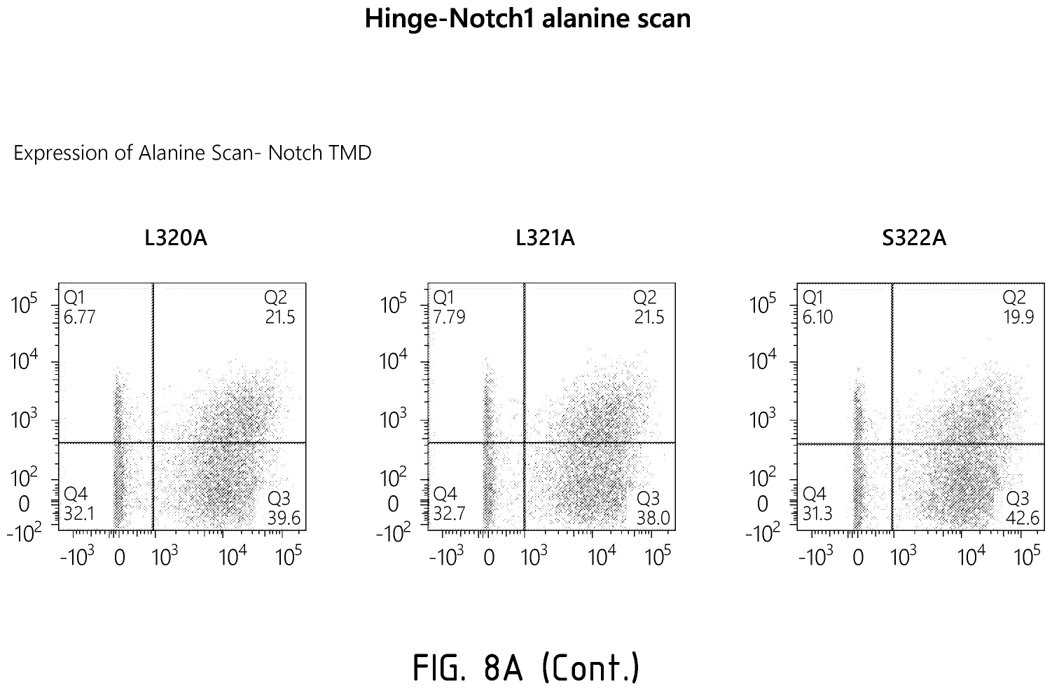

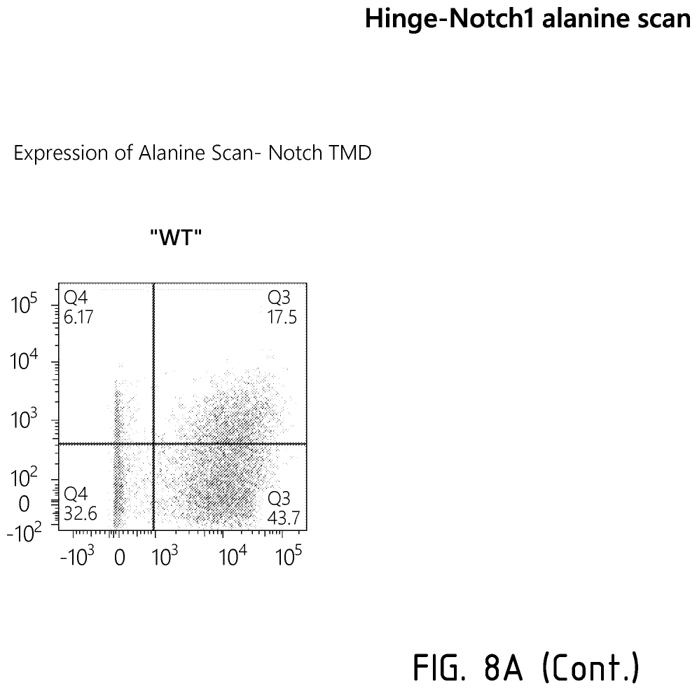

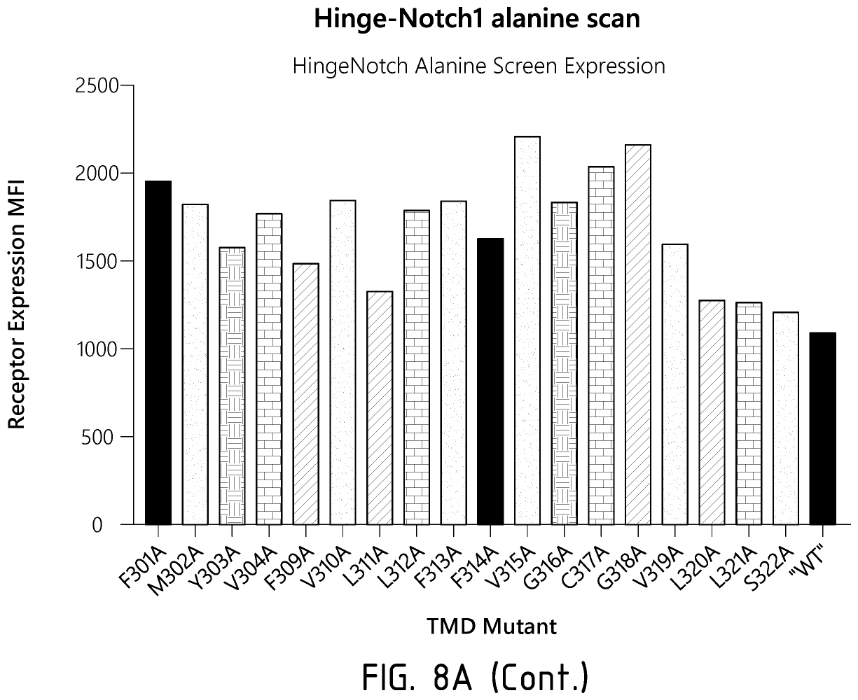

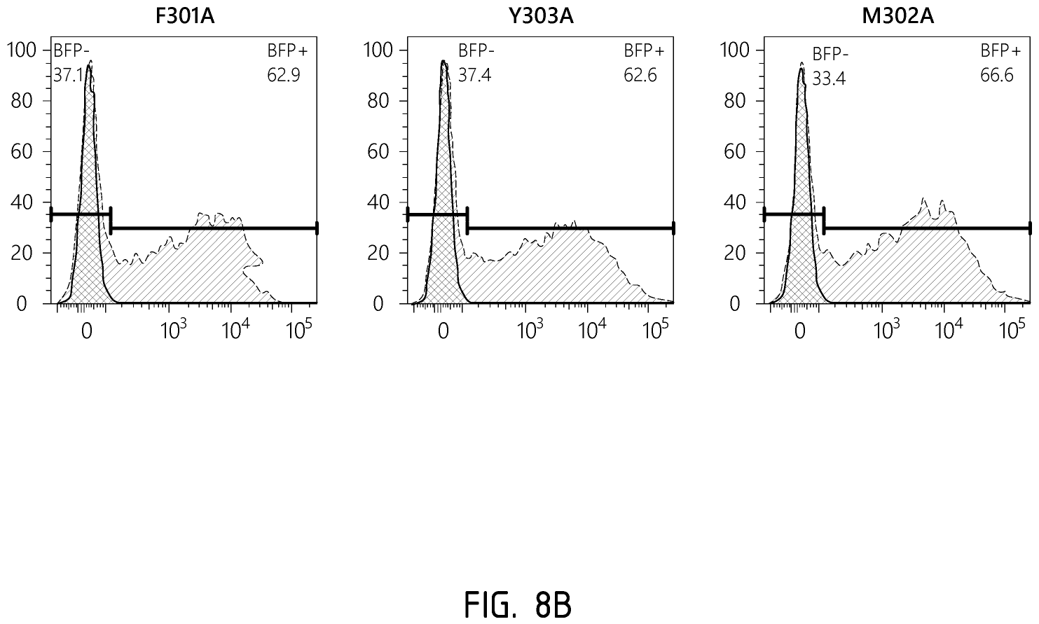

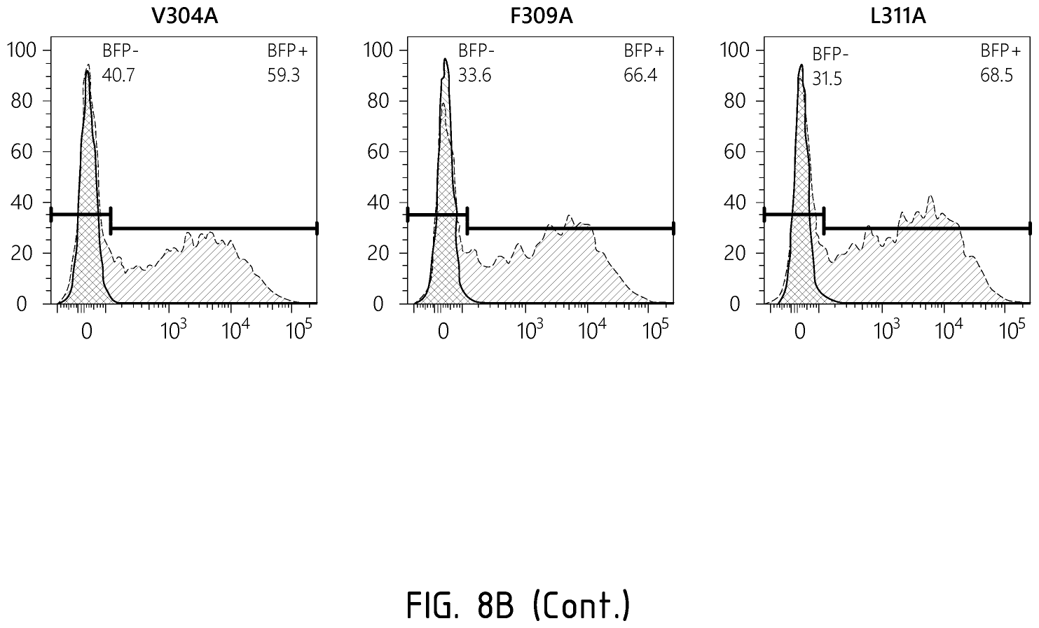

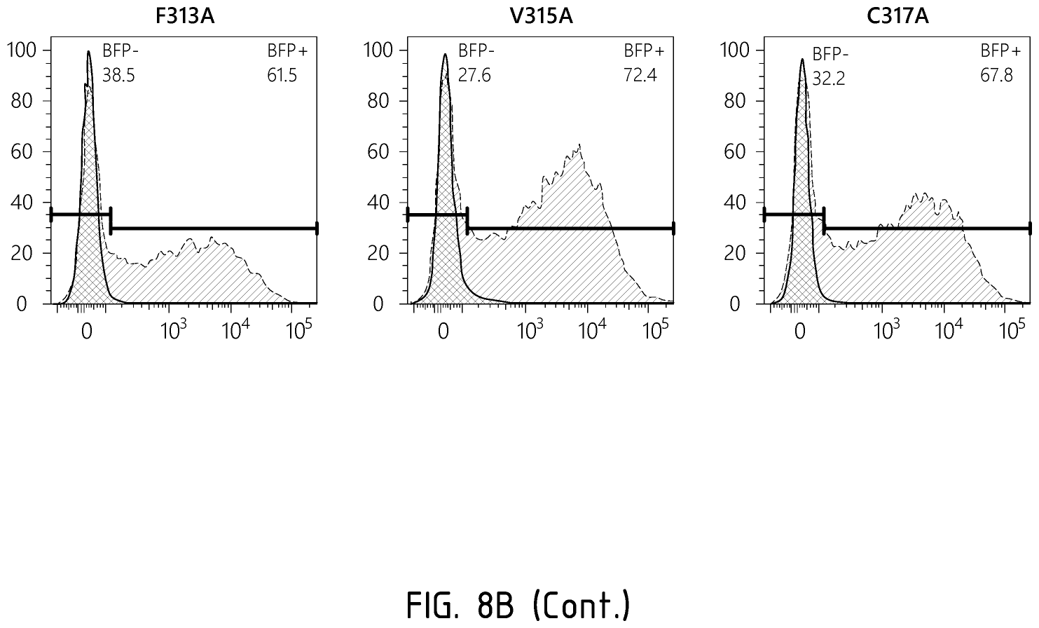

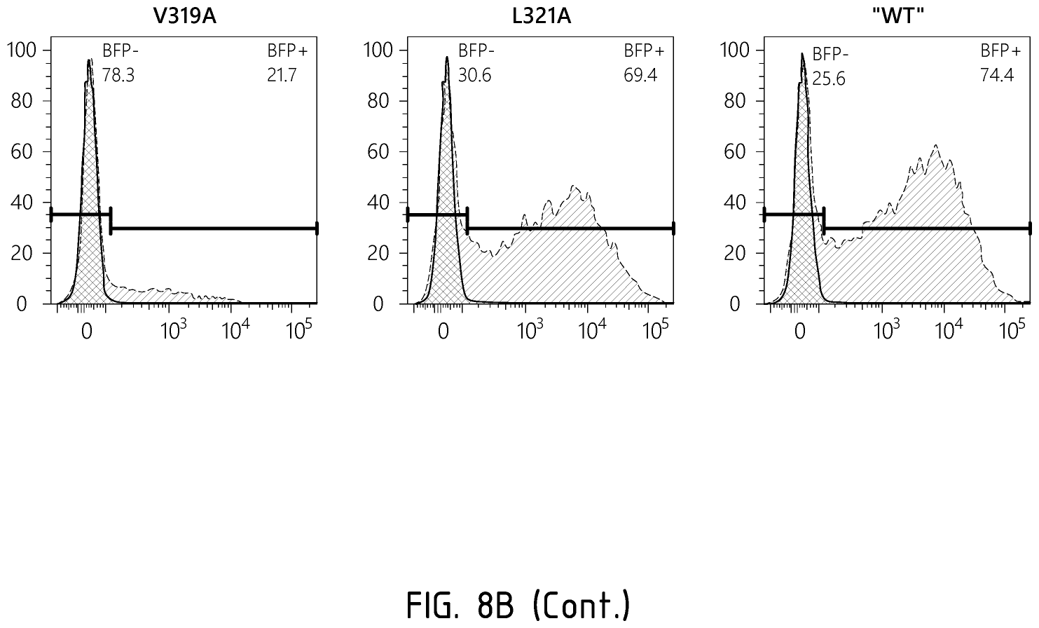

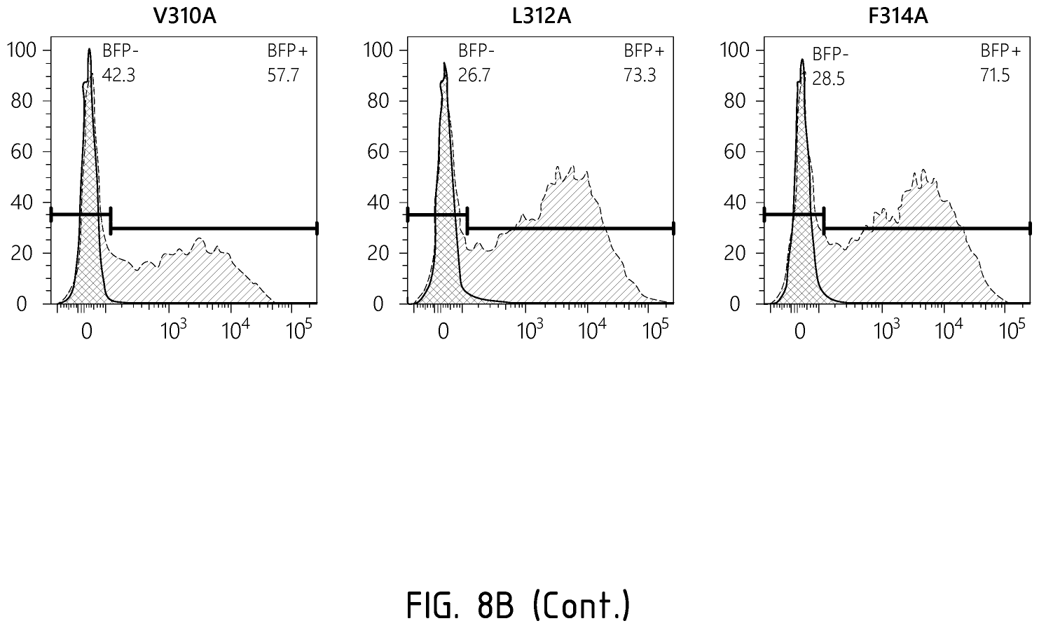

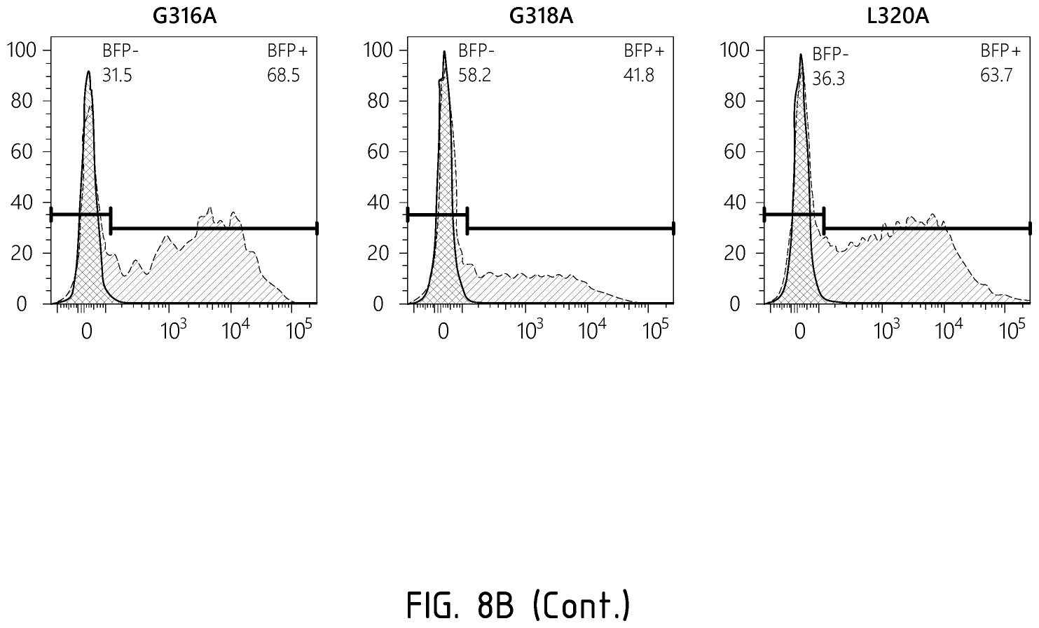

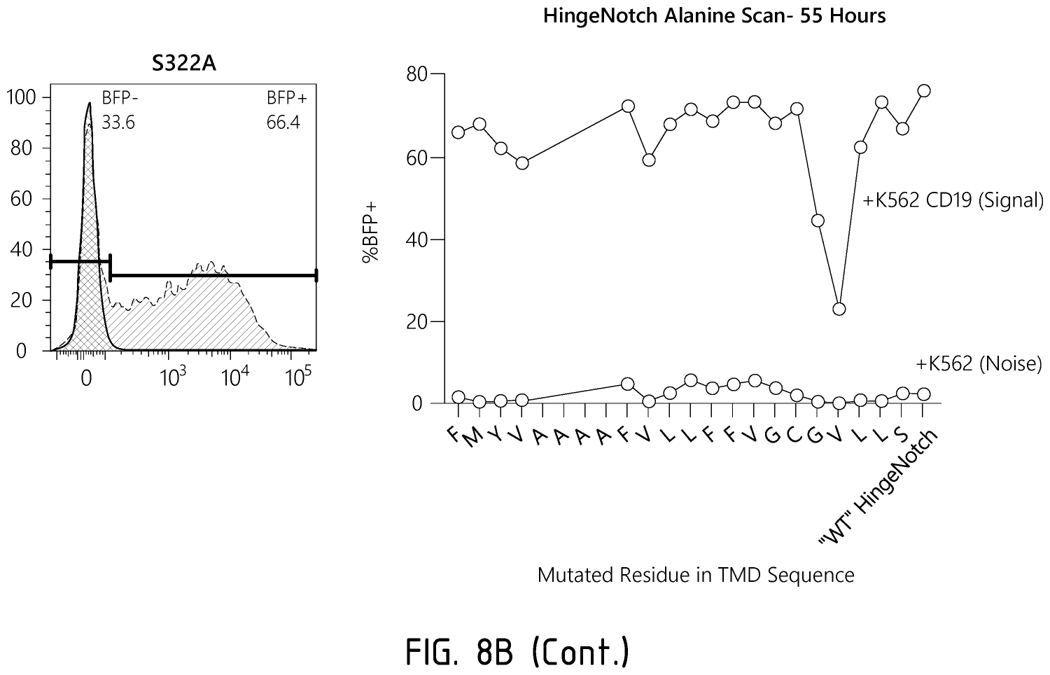

Variants with different alanine mutations in the TMD domain of the Hinge-Notch construct were prepared. Each amino acid residue from position 301 (F) through position 322 (S) in the TMD of Hinge-Notch were individually mutated to alanine. Primary human CD4+ T-cells were activated with anti-CD3/anti-CD28 Dynabeads (Gibco) and transduced with two lentiviral constructs, one expressing a TMD mutant variant, and the other containing a BFP transcriptional reporter. Cells containing both constructs were sorted for on Day 5 post initial T-cell stimulation and expanded further for activation testing. In FIG. 8A, the left panel shows relative expression of different receptors, measured by anti-myc-tag staining (y-axis), versus reporter construct marker expression (x-axis), while the right panel represents MFI quantitation of receptor expression of TMD mutant variants in double-positive cells.

As shown in FIG. 8B, T-cells expressing anti-CD19 receptors were co-cultured at a ratio of 1:1 with control CD19(−) or CD19(+) K562 cells. Transcriptional activation of an inducible BFP reporter gene was subsequently measured using a Fortessa X-50 (BD Biosciences). The left panel shows flow panels of activation profiles. The right panel represents BFP % plotted as a line graph. Results indicate the importance of the glycine (G) and valine (V) residues in the C-terminal end of the TMD.

### Example 11

This Example describes mutational analysis for the transmembrane domain (TMD) and the STS domain in Hinge-Notch constructs.

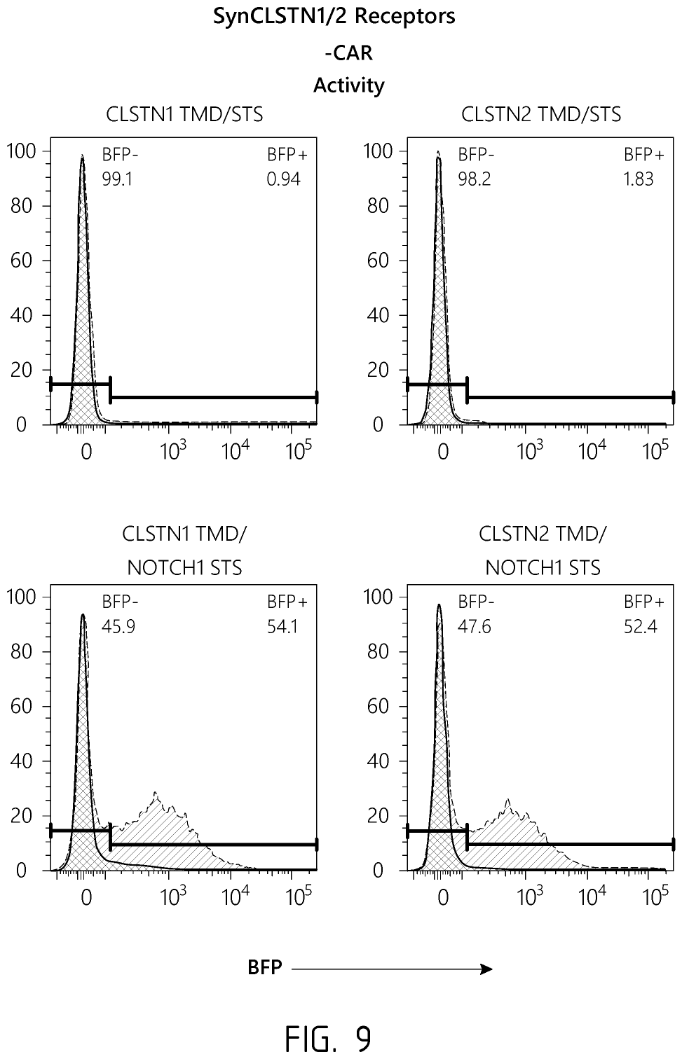

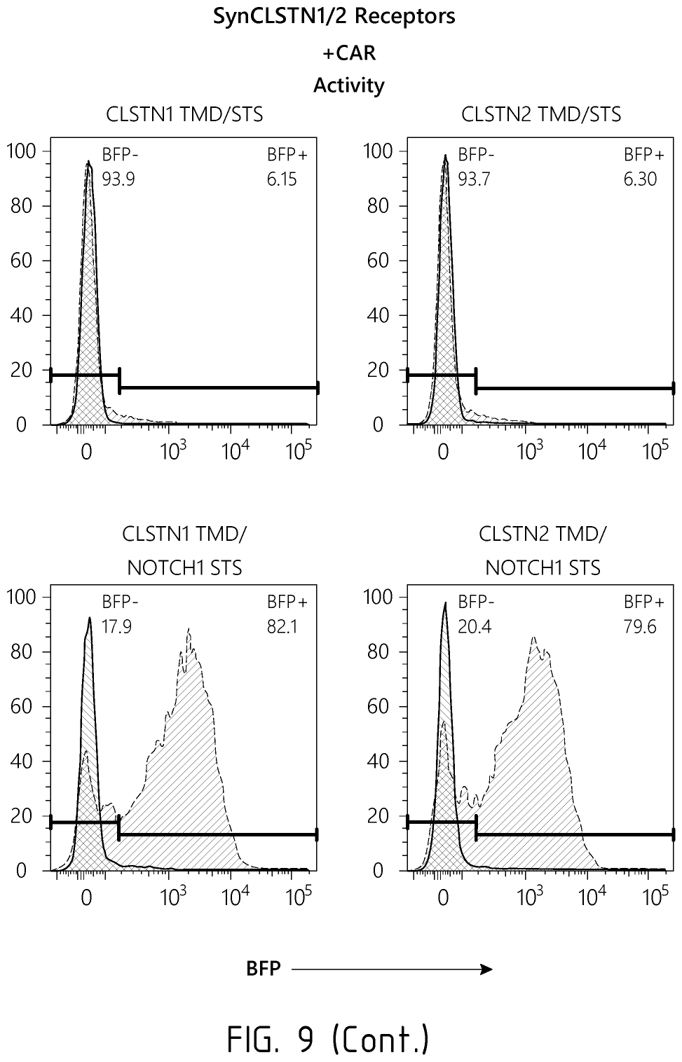

Four types of exemplary Hinge Notch receptors (SEQ ID NOS: 131-134) were using in this Example, all of which including an anti-CD19 scFv domain, a truncated CD8 Hinge domain, and a Gal4VP64 domain. For choices of STS and TMD domains, the four constructs comprise: CLSTN1 TMD and CLSTN1 STS (SEQ ID NO: 131), CLSTN2 TMD and CLSTN2 STS (SEQ ID NO: 132), CLSTN1 TMD and Notch1 STS (SEQ ID NO: 133), CLSTN2 TMD and Notch1 STS (SEQ ID NO: 134). Primary human CD4+ T-cells were activated with anti-CD3/anti-CD28 Dynabeads (Gibco) and transduced with two lentiviral constructs, one expressing a hinge receptor with TMD/STS combination as indicated, and the other a transcriptional reporter with constitutively expressed anti-ALPPL2 CAR. Cells containing both constructs were sorted for on Day 5 post initial T-cell stimulation and expanded further for activation testing. As shown in FIG. 9, 1×10⁵ double positive T-cells expressing receptors were co-cultured with: 1×10⁵ K562 cells (“−CAR” panels, blue), or 1×10⁵ CD19+K562 cells (“−CAR” panels, red). Similarly, 1×10⁵ double positive T-cells expressing receptors were tested in the presence of CAR activity by co-culture with 1×10⁵ ALPPL2+K562 cells (“+CAR” panels, blue), or 1×10⁵ ALPPL2+CD19+K562 cells (“+CAR” panels, red). Transcriptional activation of an inducible BFP reporter gene was subsequently measured using a Fortessa X-50 (BD Biosciences).

### Example 12

This Example describes experiments capable of demonstrating the function of Notch constructs and TMD variants described in the disclosure, when engineered into T cells, by measuring and comparing the production of certain cytokine(s), e.g., IL-2.

Specifically, T cells engineered with Notch TMD variants of the disclosure can be used to test ligand-triggered secretion of an engineered cytokine for autocrine and paracrine expansion of T cells. Expression profile of Notch TMD receptors with various TMD modifications may be tested. Primary human T-cells are activated with anti-CD3/anti-CD28 Dynabeads (Gibco) and transduced with two lentiviral constructs, one expressing, e.g., a CAR against the MCAM antigen, and one expressing a Notch TMD variant receptor with inducible super-IL2 under Gal4-UAS control. Cells containing both constructs are sorted on Day 5 post initial T-cell stimulation and expanded further for activation testing. Receptor expression was determined by anti-myc-tag staining.

### Example 13

This Example describes experiments capable of demonstrating that ligand-triggered expression of super-IL2 improves cell viability of CAR-T cells.

1×10⁵ double positive T-cells expressing Notch TMD variant receptors are co-cultured in media without IL-2, with no K562 cells, with CD19+K562 cells to trigger Notch constructs, with MCAM+K562 cells to trigger CAR activation, or with MCAM+ and CD19+K562 cells to trigger activation of both receptors. After 9 days the proportion of live T cells by forward and side-scatter measurements using a Fortessa X-50 (BD Biosciences) is assessed. Co-activation of both receptors results in the most viable cells, followed by Notch activation (and subsequent super-IL2 induction), CAR activation alone, and no activation of either receptor.

### Example 14

This Example describes experiments capable of demonstrating tunable proliferation of T cells with Notch TMD variants.

Primary human T-cells are activated with anti-CD3/anti-CD28 Dynabeads (Gibco) and transduced with two lentiviral constructs, one expressing, e.g., a CAR against the MCAM antigen, and one expressing a Notch TMD variant with inducible super-IL2 under Gal4-UAS control. Different Notch TMD variants are tested against a no Notch control. Similarly, primary human T-cells are generated without CAR expression. T cells are stained with CellTrace Violet (Invitrogen) according to manufacturer's protocols, co-incubated with CD19+K562 target cells in media without IL-2 and measured using a Fortessa X-50 (BD Biosciences) at different timepoints to assess proliferation by CTV signal decay.

### Example 15

This Example describes experiments capable of demonstrating tunable secretion of super-IL2 with TMD-variants of Notch variants.

Primary human T-cells are activated with anti-CD3/anti-CD28 Dynabeads (Gibco) and transduced with a lentiviral construct including a Notch TMD variant with inducible super-IL2 under Gal4-UAS control. Different Notch TMD variants are tested against a no Notch control. T cells are co-incubated with MCAM+CD19+K562 cells in media lacking IL-2, and at various timepoints, supernatant IL-2 is measured using the Instant ELISA Kit (Invitrogen) according to manufacturer's protocols with a microplate reader (Tecan). Primary human T-cells can be also generated with an additional lentiviral vector expressing, e.g., a CAR against MCAM. Enhanced uptake of IL-2 by CAR-expressing cells resulted in loss of supernatant IL2 in CAR-only and Notch TMD variant-expressing T cells.

### Example 16

This Example describes experiments capable of demonstrating that tunable secretion of super-IL2 with TMD-variants of Notch constructs enhances proliferation of bystander T cells.

Primary human T-cells are activated with anti-CD3/anti-CD28 Dynabeads (Gibco) and transduced with a lentiviral construct including a Notch TMD variant receptor with inducible super-IL2 under Gal4-UAS control. Different Notch TMD variants are tested against a no Notch control. T cells expressing such Notch TMD variants were co-incubated with “bystander” T cells stained with CellTrace Far Red (Invitrogen) expressing a CAR against MCAM or with no CAR. T cells were co-incubated with MCAM+CD19+K562 cells in media lacking IL-2, and proliferation of the bystander T cells are assessed by measuring signal decay on a Fortessa X-50 (BD Biosciences).

### Example 17

This Example describes experiments capable of testing single lentiviral vector constructs containing Notch TMD variant CAR circuits.

Primary human T-cells are activated with anti-CD3/anti-CD28 Dynabeads (Gibco) and transduced with a single lentiviral construct containing constitutively expressed Notch TMD variants with an inducible anti-MCAM CAR cassette under Gal4-UAS control. Cells are sorted for Notch receptor expression via myc-tag on Day 5 post initial T-cell stimulation and expanded further for activation testing. Different TMD variants are tested, with constitutively expressed CAR used as a control. For testing, 1×10⁵ T cells expressing Notch TMD variant receptors are co-cultured with: no additions, 5×10⁵ K562 cells, or 5×10⁴ CD19+K562 cells. Transcriptional activation of the inducible CAR is subsequently measured by a GFP tag using a Fortessa X-50 (BD Biosciences).

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

David L. Porter, M.D., Bruce L. Levine, Ph.D., Michael Kalos, Ph.D., Adam Bagg, M.D., and Carl H. June, M.D. Chimeric Antigen Receptor-Modified T Cells in Chronic Lymphoid Leukemia. N. Engl J Med. (2011) August 25; 365(8):725-33.
Gordon W R et al., The molecular logic of Notch signaling—a structural and biochemical perspective. J. Cell Sci. (2008) 121:3109-19.
Gordon W R et al., Mechanical Allostery: Evidence for a Force Requirement in the Proteolytic Activation of Notch. Dev Cell (2015) 33:729-36.
Haapasalo A I, Kovacs D M. The many substrates of presenilin/γ-secretase. J Alzheimers Dis. (2011); 25(1):3-28.
Rosmalen M., Krom M., and Merkx M. Tuning the Flexibility of Glycine-Serine Linkers To Allow Rational. Biochemistry (2017) 56:6565-6574.
Morsut L, Roybal K T, Xiong X, Gordley R M, Coyle S M, Thomson M, and Lim W A. Engineering Customized Cell Sensing and Response Behaviors Using Synthetic Notch Receptors. Cell. (2016) February 11; 164(4):780-91.
Naso M F, Tomkowicz B, Perry W L 3rd, Strohl W R. Adeno-Associated Virus (AAV) as a Vector for Gene Therapy. BioDrugs. (2017); 31(4):317-34.
Nasri M, Karimi A, Allahbakhshian Farsani M. Production, purification and titration of a lentivirus-based vector for gene delivery purposes. Cytotechnology. (2014); 66(6):1031-38.
Roybal K T, Jasper Z. Williams, Leonardo Morsut, Levi J. Rupp, Isabel Kolinko, Joseph H. Choe, Whitney J. Walker, Krista A. McNally, and Wendell A. Lim. Engineering T cells with Customized Therapeutic Response Programs Using Synthetic Notch Receptors. Cell. (2016) October 6; 167(2):419-32.
Samulski and Muzyczka. AAV-Mediated Gene Therapy for Research and Therapeutic Purposes. Annu. Rev. Virol. (2014) 1:427.
Sakuma, et al. (2012). Lentiviral vectors: basic to translational. Biochem. J. 443:603.
Watson D. J., Wolfe J. H. Viral vectors for gene therapy: methods and protocols. Totowa, N.J., USA: Humana Press; (2003). pp. 383-404.
Vidarsson G. et al., IgG subclasses and allotypes: from structure to effector functions. Frontiers Immunol. (2014) October 20; 5:520.
Zhang et al., The γ-secretase complex: from structure to function. Frontiers in Cellular Neuroscience. (2014) December 8: 427.

What is claimed is:

1. A chimeric polypeptide comprising, from N-terminus to C-terminus:
a) an extracellular binding domain having a binding affinity for a selected ligand;
b) a linking sequence having:
(i) at least about 80% sequence identity to a Notch juxtamembrane domain (JMD);
(ii) at least about 80% sequence identity to a Notch JMD wherein the LIN-12-Notch repeat (LNR) and/or a heterodimerization domain (HD) of a Notch receptor has been deleted;
(iii) at least about 80% sequence identity to a polypeptide hinge domain;
(iv) at least about 80% sequence identity to a ROBO1 JMD including at least one fibronectin repeat; or
(v) a polypeptide having about 2 to about 40 amino acids;
c) a transmembrane domain having at least about 80% sequence identity to the transmembrane domain of a Type 1 transmembrane receptor and comprising one or more ligand-inducible proteolytic cleavage sites; and
d) an intracellular domain comprising a transcriptional regulator, wherein binding of the selected ligand to the extracellular binding domain induces cleavage at the ligand-inducible proteolytic cleavage site between the transcriptional regulator and the linking sequence,
and wherein
(i) when the linking sequence has at least about 80% sequence identity to a Notch JMD, or a Notch JMD LNR and/or an HD of a Notch receptor has been deleted, the TMD is heterologous to the linking sequence; and
(ii) when the linking sequence does not have at least about 80% sequence identity to a Notch JMD or a Notch JMD wherein the LNR and/or an HD of a Notch receptor has been deleted, the transmembrane domain is not a Notch1 transmembrane domain.

2. The chimeric polypeptide of claim 1, wherein the transmembrane domain further comprises a stop-transfer sequence (STS).

3. The chimeric polypeptide of any one of claims 1 to 2, wherein the TMD comprises a polypeptide sequence having at least 80% sequence identity to a transmembrane domain from a Type 1 transmembrane receptor and comprises a γ-secretase cleavage site.

4. The chimeric polypeptide of any one of claims 1 to 3, wherein the TMD comprises a polypeptide sequence having at least 90% sequence identity to a transmembrane domain from a Type 1 transmembrane receptor and comprises a γ-secretase cleavage site.

5. The chimeric polypeptide of any one of claims 1 to 4, wherein the TMD comprises a polypeptide sequence having at least 95% sequence identity to a transmembrane domain from a Type 1 transmembrane receptor and comprises a γ-secretase cleavage site.

6. The chimeric polypeptide of any one of claims 1 to 5, wherein the Type 1 transmembrane receptor is selected from the group consisting of CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, IFNAR2, IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKHD1, NECTIN1, PCDHGC3, NRG1, LRP1B, CDH2, NRG2, PTPRK, SCN2B, Nradd, and PTPRM, and comprises a γ-secretase cleavage site.

7. The chimeric polypeptide of any one of claims 1 to 6, wherein the extracellular domain comprises an antigen-binding moiety capable of binding to a ligand on the surface of a cell.

8. The chimeric polypeptide of any one of claims 1 to 6, wherein the cell is a pathogen.

9. The chimeric polypeptide of any one of claims 1 to 8, wherein the ligand comprises a protein or a carbohydrate.

10. The chimeric polypeptide of any one of claims 1 to 9, wherein the ligand is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, and MAGE, alkaline phosphatase, placental-like 2 (ALPPL2), B-cell maturation antigen (BCMA), green fluorescent protein (GFP), enhanced green fluorescent protein (eGFP), and signal regulatory protein α (SIRPα).

11. The chimeric polypeptide of any one of claims 1 to 10, wherein the ligand is selected from cell surface receptors, adhesion proteins, integrins, mucins, lectins, tumor-associated antigens, and tumor-specific antigens.

12. The chimeric polypeptide of any one of claims 1 to 11 wherein the ligand is a tumor-associated antigen or a tumor-specific associated antigen.

13. The chimeric polypeptide of any one of claims 1 to 12, wherein the extracellular binding domain comprises the ligand-binding portion of a receptor.

14. The chimeric polypeptide of any one of claims 7 to 13, wherein the antigen-binding moiety is selected from the group consisting of an antibody, a nanobody, a diabody, a triabody, or a minibody, a F(ab′)₂ fragment, a Fab fragment, a single chain variable fragment (scFv), a single domain antibody (sdAb), or a functional fragment thereof.

15. The chimeric polypeptide of claim 14, wherein the antigen-binding moiety comprises an scFv.

16. The chimeric polypeptide of any one of claims 7 to 15, wherein the antigen-binding moiety is a tumor-associated antigen selected from the group consisting of CD19, B7H3 (CD276), BCMA (CD269), ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MUC1, NCAM, PAP, PDGFR-β, PRSS21, PSCA, PSMA, ROR1, SIRPα, SSEA-4, TAG72, TEM1/CD248, TEM7R, TSHR, VEGFR2, ALPI, citrullinated vimentin, cMet, and Axl.

17. The chimeric polypeptide of claim 16, wherein the tumor-associated antigen is CD19, CEA, HER2, MUC1, CD20, BCMA, ALPPL2, or EGFR.

18. The chimeric polypeptide of claim 17, wherein the tumor-associated antigen is CD19.

19. The chimeric polypeptide of any one of claims 1 to 18, wherein the ligand-inducible proteolytic cleavage site is a γ-secretase cleavage site.

20. The chimeric polypeptide of any one of claims 1 to 19, wherein the transcriptional regulator comprises a transcriptional activator, or a transcriptional repressor.

21. The chimeric polypeptide of any one of claims 1 to 20, wherein the ICD comprises a nuclear localization sequence and a transcriptional regulator sequence selected from Gal4-VP16, Gal4-VP64, tetR-VP64, ZFHD1-VP64, Gal4-KRAB, and HAP1-VP16.

22. The chimeric polypeptide of any one of claims 1 to 21, further comprising a signal sequence, a detectable label, a tumor-specific cleavage site, a disease-specific cleavage site, or a combination thereof.

23. The chimeric polypeptide of any one of claims 2 to 22, wherein the STS comprises an amino acid sequence having at least 80% sequence identity to SEQ ID NO: 96, 135, 136, or 137.

24. The chimeric polypeptide of any one of claims 1 to 23, wherein the linking sequence comprises an amino acid sequence having at least 80% sequence identity to any one of SEQ ID NOS: 97-99 and 138-148.

25. The chimeric polypeptide of claim 24, wherein the linking sequence comprises an amino acid sequence having at least 90% sequence identity to any one of SEQ ID NOS: 97-99 and 138-148.

26. The chimeric polypeptide of claim 25, wherein the linking sequence comprises an amino acid sequence having at least 95% sequence identity to any one of SEQ ID NOS: 97-99 and 138-148.

27. The chimeric polypeptide of claim 26, wherein the linking sequence comprises an amino acid sequence substantially identical to any one of SEQ ID NOS: 97-99 and 138-148.

28. The chimeric polypeptide of any one of claims 1 to 27, wherein the TMD comprises an amino acid sequence having at least 80% sequence identity to any one of SEQ ID NOS: 1-94.

29. The chimeric polypeptide of claim 28, wherein the TMD comprises an amino acid sequence having at least 90% sequence identity to any one of SEQ ID NOS: 1-94.

30. The chimeric polypeptide of any one of claims 1 to 29, wherein the TMD comprises an amino acid sequence having at least 95% sequence identity to any one of SEQ ID NOS: 1-94.

31. The chimeric polypeptide of claim 30, wherein the TMD comprises an amino acid sequence substantially identical to any one of SEQ ID NOS: 1-94.

32. The chimeric polypeptide of any one of claims 1 to 31, wherein:
a) the linking sequence comprises an amino acid sequence having at least 80% sequence identity to any one of SEQ ID NOS: 97-99 and 138-148;
b) the TMD comprises an amino acid sequence having at least 80% sequence identity to any one of SEQ ID NO: 1-94; and
c) the STS comprises an amino acid sequence having at least 80% sequence identity to SEQ ID NO: 96, 135, 136, or 137.

33. A recombinant nucleic acid comprising a nucleotide sequence encoding a chimeric polypeptide according to any one of claims 1 to 32.

34. The recombinant nucleic acid of claim 33, wherein the nucleotide sequence is incorporated into an expression cassette or an expression vector.

35. The recombinant nucleic acid of claim 34, wherein the expression vector is a viral vector.

36. The recombinant nucleic acid of claim 35, wherein the viral vector is a lentiviral vector, an adenovirus vector, an adeno-associated virus vector, or a retroviral vector.

37. A recombinant cell comprising:
a) a chimeric polypeptide according to any one of claims 1 to 32; and/or
b) a recombinant nucleic acid according to any one of claims 33 to 36.

38. The recombinant cell of claim 37, wherein the cell is a mammalian cell.

39. The recombinant cell of claim 38, wherein the mammalian cell is an immune cell, a neuron, an epithelial cell, an endothelial cell, or a stem cell.

40. The recombinant cell of claim 39, wherein the immune cell is a B cell, a monocyte, a natural killer cell, a basophil, an eosinophil, a neutrophil, a dendritic cell, a macrophage, a regulatory T cell, a helper T cell, a cytotoxic T cell, or other T cell.

41. The recombinant cell of any one of claims 37 to 40, further comprising:
a) a second chimeric polypeptide according to any one of claims 1 to 32; and/or
b) a second nucleic acid according to any one of claims 33 to 36;
wherein the first chimeric polypeptide and the second chimeric polypeptide do not have the same sequence, and/or the first nucleic acid or the second nucleic acid do not have the same sequence.

42. The recombinant cell of claim 41, wherein the chimeric polypeptide modulates the expression and/or activity of the second chimeric polypeptide.

43. The recombinant cell of any one of claims 37 to 42, further comprising an expression cassette encoding a protein of interest operably linked to a promoter, wherein expression of the protein is modulated by the transcriptional regulator encoded by the chimeric receptor.

44. The recombinant cell of claim 43, wherein the protein of interest is heterologous to the cell.

45. The recombinant cell of claim 43 or 44, wherein the promoter is GAL4.

46. The recombinant cell of claim 43 or 45, wherein the protein of interest is a cytokine, a cytotoxin, a chemokine, an immunomodulator, a pro-apoptotic factor, an anti-apoptotic factor, a hormone, a differentiation factor, a de-differentiation factor, an immune cell receptor, or a reporter.

47. A cell culture comprising a recombinant cell according to any one of claims 37 to 46, and a culture medium.

48. A pharmaceutical composition comprising a pharmaceutically acceptable carrier, and one or more of the following:
a) a recombinant nucleic acid according to any one of claims 33 to 36; or
b) a recombinant cell according to any one of claims 37 to 46.

49. The pharmaceutical composition of claim 48, wherein the composition comprises a recombinant nucleic acid according to any one of claims 33 to 36, and a pharmaceutically acceptable carrier.

50. The pharmaceutical composition of claim 49, wherein the recombinant nucleic acid is encapsulated in a viral capsid or a lipid nanoparticle.

51. A method for modulating an activity of a cell, the method comprising:
a) providing a recombinant cell according to any one of claims 37 to 46; and
b) contacting the recombinant cell with the selected ligand, wherein binding of the selected ligand to the extracellular binding domain induces cleavage of a ligand-inducible proteolytic cleavage site and releases the transcriptional regulator, wherein the released transcriptional regulator modulates an activity of the recombinant cell.

52. The method of claim 51, the contacting is carried out in vivo, ex vivo, or in vitro.

53. The method of any one of claims 51 to 52, wherein the activity of the cell is selected from the group consisting of: expression of a selected gene of the cell, proliferation of the cell, apoptosis of the cell, non-apoptotic death of the cell, differentiation of the cell, de-differentiation of the cell, migration of the cell, secretion of a molecule from the cell, cellular adhesion of the cell, and cytolytic activity of the cell.

54. The method of any one of claims 51 to 53, wherein the released transcriptional regulator modulates expression of a gene product of the cell.

55. The method of any one of claims 51 to 54, wherein the released transcriptional regulator modulates expression of a heterologous gene product.

56. The method of any one of claims 54 to 55, wherein the gene product of the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen derived protein, a proliferation inducer, a receptor, an RNA guided nuclease, a site-specific nuclease, a T cell receptor, a toxin, a toxin-derived protein, a transcriptional regulator, a transcriptional activator, a transcriptional repressor, a translational regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

57. The method of any one of claims 51 to 56, wherein the released transcriptional regulator modulates differentiation of the cell, and wherein the cell is an immune cell, a stem cell, a progenitor cell, or a precursor cell.

58. A method for inhibiting an activity of a target cell in an individual, the method comprising administering to the individual an effective number of the recombinant cell according to any one of claims 37 to 46, wherein the recombinant cell inhibits an activity of the target cell in the individual.

59. The method of claim 58, wherein the target cell is an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell.

60. The method of claim 58, wherein the target cell is a pathogenic cell.

61. A method for the treatment of a health condition in an individual in need thereof, the method comprising: administering to the individual a first therapy comprising an effective number of the recombinant cell according to any one of claims 37 to 46, wherein the recombinant cell treats the health condition in the individual.

62. The method of claim 61, further comprising administering to the individual a second therapy.

63. The method of claim 62, wherein the second therapy is selected from the group consisting of chemotherapy, radiotherapy, immunotherapy, hormonal therapy, or toxin therapy.

64. The method of any one of claims 61 to 63, wherein the first therapy and the second therapy are administered together, in the same composition or in separate compositions.

65. The method any one of claims 62 to 64, wherein the first therapy and the second therapy are administered simultaneously.

66. The method of any one of claims 62 to 63, wherein the first therapy and the second therapy are administered sequentially.

67. The method of claim 66, wherein the first therapy is administered before the second therapy.

68. The method of claim 66, wherein the first therapy is administered after the second therapy.

69. The method of claim 66, wherein the first therapy and the second therapy are administered in rotation.

70. A system for modulating an activity of a cell, inhibiting a target cancer cell, or treating a health condition in an individual in need thereof, wherein the system comprises one or more of the following:
a) a chimeric polypeptide according to any one of claims 1 to 32;
b) a recombinant nucleic acid according to any one of claims 33 to 36;
c) a recombinant cell according to any one of claims 37 to 46; and
d) a pharmaceutical composition according to any one of claims 48 to 50.

71. A method for making the recombinant cell according to any one of claims 37 to 46, comprising:
a) providing a cell capable of protein expression; and
b) contacting the provided cell with a recombinant nucleic acid according to any one of claims 33 to 36.

72. The method of claim 71, wherein the cell is obtained by leukapheresis performed on a sample obtained from a human subject, and the cell is contacted ex vivo.

73. The method of claim 71, wherein the recombinant nucleic acid is encapsulated in a viral capsid or a lipid nanoparticle.

74. The use of one or more of the following for the treatment of a health condition:
a) a chimeric polypeptide according to any one of claims 1 to 32;
b) a recombinant nucleic acid according to any one of claims 33 to 36;
c) a recombinant cell according to any one of claims 37 to 46; and
d) a composition according to any one of claims 48 to 50.

75. The use of claim 74, wherein the health condition is cancer.

76. The use of the invention of any one of claims 1 to 75, for the manufacture of a medicament for the treatment of a health condition.
